SEQUENCE LISTING (224 sequences)

SEQ ID NO: 1 - DNA, 792 bp, Babesia microti
cactctttttaatgagcggtgctgtctttgcaagtgataccgatcccgaagctggtgggcctagtgaagctggtgggcctagtggaactgttgggcccagtgaagctggtgggcctagtgaagctggtgggcctagtggaactggttggcctagtgaagctggtgggcctagtgaagctggtgggcctagtgaagctggtgggcctagtgaagctggtgggcctagtggaactggttggcctagtggaactggttggcctagtgaagctggttggtctagtgaacgatttggatatcagcttcttccgtattctagaagaatagttatatttaatgaagtttgtttatcttatatatacaaacatagtgttatgatattggaacgagatagggtgaacgatggtcataaagactacattgaagaaaaaaccaaggagaagaataaattgaaaaaagaattggaaaaatgttttcctgaacaatattcccttatgaagaaagaagaattggctagaatatttgataatgcatccactatctcttcaaaatataagttattggttgatgaaatatcaaacaaggcctatggtacattggaaggtccagctgctgataattttgaccatttccgtaatatatggaagtctattgtacttaaagatatgtttatatattgtgacttattattacaacatttaatctataaattctattatgacaataccgttaatgatatcaagaaaaattttgacgaatccaaatctaaagctttagttttgagggataagatca

SEQ ID NO: 2 - DNA, 2732 bp, Babesia microti
aaaccctaaaccctaaaccctaaaccctaaaccctaaacccctaaaccctaaaccctaaaccctaaaccctaaaccctaaaaccctaaaccctaaaccctaaaccctaaaccctaaaccctaaaccctaaaccctaaaccctaaaccctaaaccctaaaccctaaaccctaaaccctaaaccctaaaccctaaaccctaaaccctaaaccctaaacccctaaaccctaaaccctaaaccctaaaccctaaaccctaaaccctaaaccctaaaccctaaaccctaaaccctaaaccctaaaccctaaaccctaaaccctaaaccctaaaccctaaaaccctaaaccctaaaccctaaaccctaaaccctaaaccctaaacccctaaaccctaaaccctaaaccctaaaccctaaaccctaaacccctaaaccctaaacccctaaaccctaaaccctaaaccctaaaccctaaaccctaaaccctaaaccctaaaccctaaaccctaaaccctaaacccctaaaccctaaaccctaaaccctaaaccctaaaccctaaaccctaaaccctaaaccctaaccctaaccctaaccctaaccctaacctagccttcattgacgtctatccccaatcttagaaaaatcttcaaatcgattctagaataactggaaacaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaagtgataccgatcccgaagctggtgggcctagtgaagctggtgggcctagtggaactgttgggcccagtgaagctggtgggcctagtgaagctggtgggcctagtggaactgttgggcccagtgaagctggtgggcctagtgaagctggtgggcctagtggaactggttggcctagtgaagctggtgggcctagtgaagctggtgggcctagtggaactgttgggcccagtgaagctggtgggcctagtgaagctggtgggcctagtggaactggttggcctagtgaagctggtgggcctagtgaagctggtgggcctagtgaagctggtgggcctagtgaagctggtgggcctagtggaactggttggcctagtggaactggttggcctagtgaagctggttggtctagtgaacgatttggatatcagcttcttccgtattctagaagaatagttatatttaatgaagtttgtttatcttatatatacaaacatagtgttatgatattggaacgagatagggtgaacgatggtcataaagactacattgaagaaaaaaccaaggagaagaataaattgaaaaaagaattggaaaaatgttttcctgaacaatattcccttatgaagaaagaagaattggctagaatatttgataatgcatccactatctcttcaaaatataagttattggttgatgaaatatcaaacaaggcctatggtacattggaaggtccagctgctgataattttgaccatttccgtaatatatggaagtctattgtacttaaagatatgtttatatattgtgacttattattacaacatttaatctataaattctattatgacaataccgttaatgatatcaagaaaaattttgacgaatcctggacacagacattaaaagaataagcctgcttgggggtttctgggcatctcttcatgagtgccagtcacacaactcttctgtgagccttctacaataaggactttgtgtgcttcgatatttttttagactaaagtgaactctctcctccacctttggcttcagttagttatttcaaatggcaaaagttattaaaaattccagtgtggaaactggcttaaccaacaggaaaggggttttgaggtcgcatcactaagcatcaagtttaacaccaacatgcctggaggattggcttagccggttgctagggcaggcctgtggcagggttcttatcccagctattaacgctcccttcccactcctccaagtcctgcaagtcctggatacagtgaaatgtaattgcatatcccatatcctttgctagtatcaaatggataaaacccaaaatggagtcataccaaatgatctcatgtatacaatacctgaatagtcttgaactgatgcactgttagatagtatgcacttactcttcagctattcatagtgtgcctctgcacagtgatggaaaagaggagcactgggggagctcggttttcaagggacaaaggagaataagacacacaaagaaatccaaggtagagcagagaaaggatggagacacagaaggtttgcaggaacaggaagcgaaggatgctccagtctgagggggaggggaaagagagcctcttgagtagccagcacctgaacttggcctggaagcttggtggataaggcaggataaaggaggtgtggcctctttggtatcctcccattgataaaggagctccctgacccttcactagaccatcatcagtcctatggttcttagaccaatagaacacaatggaattgatttgttccactttccaggttaagacttacagtcagggaagtttgtttttcttgcc

SEQ ID NO: 3 - DNA, 2430 bp, Babesia microti
aactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaagtgataccgatcccgaagctggtgggcctagtgaagctggtgggcctagtggaactgttgggcccagtgaagctggtgggcctagtgaagctggtgggcctagtggaactggttggcctagtgaagctggtgggcctagtgaagctggtgggcctagtgaagctggtgggcctagtgaagctggtgggcctagtggaactggttggcctagtggaactggttggcctagtgaagctggttggtctagtgaacgatttggatatcagcttcttccgtattctagaagaatagttatatttaatgaagtttgtttatcttatatatacaaacatagtgttatgatattggaacgagatagggtgaacgatggtcataaagactacattgaagaaaaaaccaaggagaagaataaattgaaaaaagaattggaaaaatgttttcctgaacaatattcccttatgaagaaagaagaattggctagaatatttgataatgcatccactatctcttcaaaatataagttattggttgatgaaatatcaaacaaggcctatggtacattggaaggtccagctgctgataattttgaccatttccgtaatatatggaagtctattgtacttaaagatatgtttatatattgtgacttattattacaacatttaatctataaattctattatgacaataccgttaatgatatcaagaaaaattttgacgaatccaaatctaaagctttagttttgagggataagatcactaaaaaggatggagattataacactcattttgaggacatgattaaggagttgaatagtgcagcagaagaatttaataaaattgttgacatcatgatttccaacattggggattatgatgagtatgacagtattgcaagtttcaaaccatttctttcaatgatcaccgaaatcactaaaatcaccaaagtttctaatgtaataattcctggaattaaggcactaactttaaccgtttttttaatatttattacaaaatagatgtaataccagatgtatacattattatatattacaaaatttacacattatttatgtatgaacgaacgaacatctcagtcttaaatgaagaaattgggataaatatggaaatagattaaagtaacatgagaaagatgaatataatattagaatatgaaatttaacagaaataaaatgaagtaaaagagtgtattttgtaataatttataataaattagtatacaatgattatattacagatgactattgattattgtatcaattaaatattgattattaatgatatcatatatgtatatgttaatgattgatttgttatacgttgtgaatatgttatataatgacatactataataattaatataatgtagaggatattttttttaatagtatttaatgaatattatagttataattataataatgtagataaaaatgacattaatttgaatgtttaaattgaaatgtatgtaaaaatatgtatttataatctgaattgattaataatataatattctacaattaattatttttgtaattataataattgattatattaatctttgaattattataaataatattatacttcattaaattatttcacataaatttccaaattattatcctttatcttaatgttatccaattttacacatctttcttcattacaatatttttttactaatcctgtatgctcatattcatattctttagaaatataacgaaaattagatgtaacttcgccacttacaagtaaactaccatcaatataataataatgaataccattcatgtccgtatattctttatattttttatcatattttattttgtgattattccattcatttgtatcattattcaatgagagaaataatagcagaaagatccttctatagaaacataaaattcaattaatactggattattatgtttgcaagtatagatgtttaaatcaataacactaccagttggtaatttagcattgtcatcaaattcaattatataatcagaaattttgattttatcaattttattcggatgtgataatttattttgttctgattcatcgatcatgtatacaaatactattgttaaaggttccctatccttataattaaagtggccaataagattggcattaattacattagtagtgtgtgtatttgtaatagtatcattagtggtactgacagttgttataggttttgatttccataatgaaacatcatttttatctacacaataca

SEQ ID NO: 4 - DNA, 1991 bp, Babesia microti
aatgtacaagatcaaaatttctgattatataattgaatttgatgacaatgctaaattaccaactgataatgttattggtatatccatctatacttgtgaacacaataatccagtattaattgaattttatgtttctaaaaaaggatcaatctgctattatttctactcaatgaataatgatacaaataaatggaataatcacaaaataaaatatgacaaaagatttaatgaacatactgacatgaatggtattcattattattatattgatggtagtttacttgcgagtggcgaagttacatctaattttcgttatatttctaaagaatatgaatatgagcatacagaattagcaaaagagcattgcaagaaagaaaaatgtgtaaatgtggataacattgaggataataatttgaaaatatatgcgaaacagtttaaatctgtagttactactccagctgatgtagcgggtgtgtcagatggattttttatacgtggccaaaatcttggtgctgtgggcagtgtaaatgaacaacctaatactgttggtatgagtttagaacaattcatcaagaacgagctttattcttttagtaatgaaatttatcatacaatatctagtcaaatcagtaattctttcttaataatgatgtctgatgcaattgttaaacatgataactatattttaaaaaaagaaggtgaaggctgtgaacaaatctacaattatgaggaatttatagaaaagttgaggggtgctagaagtgaggggaataatatgtttcaggaagctctgataaggtttaggaatgctagtagtgaagaaatggttaatgctgcaagttatctatccgccgcccttttcagatataaggaatttgatgatgaattattcaaaaaggccaacgataattttggacgcgatgatggatatgattttgattatataaatacaaagaaagagttagttatacttgccagtgtgttggatggtttggatttaataatggaacgtttgatcgaaaatttcagtgatgtcaataatacagatgatattaagaaggcatttgacgaatgcaaatctaatgctattatattgaagaaaaagatacttgacaatgatgaagattataagattaattttagggaaatggtgaatgaagtaacatgtgcaaacacaaaatttgaagccctaaatgatttgataatttccgactgtgagaaaaaaggtattaagataaacagagatgtgatttcaagctacaaattgcttctttccacaatcacctatattgttggagctggagttgaagctgtaactgttagtgtgtctgctacatctaatggaactgaatctggtggagctggtagtggaactggaactagtgtgtctgctacatctactttaactggtaatggtggaactgaatctggtggaacagctggaactactacgtctagtggaacttggtttggaaaatgaaaaattagctctagaaacactttattgttaatttttaaaaacctattgaaaaatcagattgtaaaacataattccacttctaaccatgctatgatttaactaatcaggacaaaaagaaagcataatcaacattattcattcagtgatggtgacataattcagagaatgtggcaattgcctcttgaagaccagagttccatccacaggacccacatggttaaaggagagagctaactcctgaaagttgtcctctgactaacacattcaacttttgagtgtctcatttatgtgttggcttctgtctaatgtgggaaaatcattaagggctcttaaatcagatcctcattctctctattaataaactatgtgataacatccttcagctatgaaaatgtcaggagagagtcaggaaaatggaagatattgttcaggacttaactaggtggtggcacacagttcctttacacagattcctcaggacaagttttaggtgaggttttgatctatcctg

SEQ ID NO: 5 - DNA, 1271 bp, Babesia microti
ttcactaggccaaccagcttcactaggccaaccagcttcactaggccaaccagcttcactaggccaaccagcttcactaggccaaccagcttcactaggccaaccagttccactaggcccaccagcttcactaggcccaccagcttcactaggcccaccagcttcactaggccaaccagttccactaggcccaccagcttcactaggcccaccagcttcactaggcccaccagcttcactaggcccaccagcttcactaggcccaccagcttcactaggcccaccagcttcactaggcccaccagcttcactaggcccaccagcttcactaggcccaacagttccactaggcccaccagcttcgcgatcggtatcacctgcaaagacagcaccgctcattaaaaagagtgtaatataaggaactaatattgatttaaatgacaccatctttataaaccatagttattggtacattattagtacattattggtatatgattggtacgtggtagtgattgtggtgctgcatctagttgtcatcaatgtgcatacatcctaactaataagctaataagctaataagcagttatacaatttctgataattgcttccagttattctagaatcgatttgaagatttttctaagattggggatagacgtcaatgaaggctaggttagggttagggttagggttagggttagggtttagggtttagggtttagggtttagggtttagggttagggtttagggtttagggtttagggtttaggctcccaagttgtcccgtgaaagggccgtgtctttgataaattttgccgtcctgtacgtttcctttctagaatgcacaaaaacaagaatttggcagctagaaacatcgttaatcacctcttggtagagaatttcgttgattgcgttgaaacgtttgatagccttcttctccttcacgccataatacacctgctccaagggcacaggcctaaagtggctgccaaagtagaaaagccctcggtctagattaacagtgagaaatctagccacgtcttcgtagtttggaagcgtggccgatagaccaactagccttacgcgttcgggcctctgactcaggcgggccacaatagcctccagcactggacccctagtgtcatggagtaggtgtatttcatcaattataaccaatctaagccgctcaagcaggggctcattgcctgttttacgtgtaactacgtcaaacttctctggcgtagttacaattatatgcgttttctca

SEQ ID NO: 6 - DNA, 1821 bp, Babesia microti
taaaccctaaacccctaaaccctaaaccctaaaccctaaaccctaaaccctaaacccctaaaccctaaaccctaaaccctaaaccctaaaccctaaccctaaaccctaaaccctaaaccctaaaccctaaaccctaaccctaaccctaaccctaaccctaacctagccttcattgacgtctatccccaatcttagaaaaatcttcaaatcgattctagaataactggaagcaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactggttggcctagtgaagctggttggcctagtgaagctggttggcctagtgaagctggttggcctagtgaagctggttggcctagtgaagctggttggcctagtgaacgatttggatatcagcttctttggtattctagaagaatagttatatttaatgaaatttatttatctcatatatacgaacatagtgttatgatattggaacgagatagggtgaacgatggtcataaagactacattgaagaaaaaaccaaggagaagaataaattgaaaaaagaattggaaaaatgttttcctgaacaatattcccttatgaagaaagaagaattggctagaataattgataatgcatccactatctcttcaaaatataagttattggttgatgaaatatccaacaaagcctatggtacattggaaggtccagctgctgatgattttgaccatttccgtaatatatggaagtctattgtacctaaaaatatgtttctatattgtgacttattattaaaacatttaatccgtaaattctattgtgacaataccattaatgatatcaagaaaaattttgacgacatagagaaattgggctgttttcaagctagaagcttcctccctgttaactaatgtattcatggtgccagaaggtgctatgcaggttgctagggaatcaaattcatcaatagtcctgcccaagagtagtgtgttaactggcggtgcaagatgtgccctttgatgcagtagtggcatgcttgtttgtggggtaacccagtgctttctgattgaggtctactccacaggaggaatagatacctgcttctgtaaacttggtcaaaacttatgactgcacatgaagacagagtggaaaagacctgaaaacacacacggggtcaggactgaggaagacagggttagtattagagagatttggggaaaaaaagagttagcaaatatagagtgtgatagtctaatggggggatgaatggtatcaaaatgaattatttatatgtataaaactgacaattttttaattgtgaaaaggaatgcaatccgacccatctgggggaattctagctagcatcagtgagagaagaggcaaggtgttaggaaatcgtgcagaacatgctcatccaggctttatttctccatttacatctagag

SEQ ID NO: 7 - DNA, 4223 bp, Babesia microti
catcacaattattggctgttacatcactatagtgctgtatgtaaaaaattataaagtgtgacatcattataatgcaatatgacatcacaattatatactgtgacttcactatcttgcactttaacatcacaattatacattgtgacatcaatatactgcactatgacatcacgattattgactgtgacatcaatacattctctatgaacacagttatacactctgacatcactagcttgcactgtgacatgacaattaaaaactgtgacatcaatataatggactgtgacctacaattattcactgtgaaaccacaacactgcaattgtgtataattgggatgggtactgatctgctgcccgaggctcaatagattacctaggcctcctcactgacacccacattcagggggtcttgatcagtcccatgatggattcccaggctgatgcctgggattcaagagttaacctttgtctggtcagctctttctgggggttaaacggattaaatgttttaataataagtcacaatatagaaacatatttttaggtacaatagacttccatatattacggaaatggtcaaaatcatcagcagctggaccttccaatgtaccataggctttgttggatatttcatcaaccaataacttatattttgaagagatagtggatgcattatcaattattctagccaattcttctttcttcataagggaatattgttcaggaaaacatttttccaattcttttttcaatttattcttctccttggttttttcttcaatgtagtctttatgaccatcgttcaccctatctcgttccaatatcataacactatgttcgtatatatgagataaataaatttcattaaatataactattcttctagaataccaaagaagctgatatccaaatcgttcactaggccaaccagcttcactaggccaaccagcttcactaggccaaccagcttcactaggccaaccagcttcactaggccaaccagcttcactaggccaaccagcttcactaggcccaccagcttcactaggcccaccagcttcactaggcccaccagcttcactaggcccaacagttccactaggcccaccagcttcactaggcccaccagcttcactaggcccaccagcttcactaggcccaccagcttcactaggcccaccagcttcactaggcccaccagcttcactaggcccaccagcttcactaggcccaacagttccactaggcccaccagcttcgcgatcggtatcacctgcaaagacagcaccgctcattaaaaagagtgtaatataaggaactaatattgatttaaatgacaccatctttataaaccatagttattggtacattattagtacattattggtatatgattggtacgtggtagtgattgtggtgctgcatctagttgtcatcaatgtgcatacatcctaactaataagctaataagctaataagcagttatacaatttctgataattgcttccagttattctagaatcgatttgaagatttttctaagattggggatagacgtcaatgaaggctaggttagggttagggttagggttagggttagggtttagggtttagggtttagggtttagggtttagggttagggtttagggtttagggtttagggtttagggtttaggggtttagggtttagggtttagggtttagggtttagggtttagggtttagggaaggctgagaaccactgacttagactttccaagactttgtcatcttatgacttgccggttgcctcgtttctccacacagcaacctatgttctctcttattacagtttctgtgggacatgtcatgcttccagcttcgagaatggaagcctattgtcttaatgggtgagcaaagtgggcccattcattaatcacagactaatccaaaaggaaatgtgacacctgacctaagtccgaccaataggagccaggaaagctcacttctggaattgtgacttagatatcacggatgcatacagactctttttcctgctgaaacaaatggtgaggacctgtccacccttgtgggaagcttgcagtgtaagattctaatccatattggggaaataaggctgagaagagagagttccaggccttgtgacagaatctaatccctggataaagtctctctttttacaaagaacatcagtgttgcaagctccaaattcctgttcttactttcttgagtctgttttctttatgtataacccaaagcactttaactgacacagctgtgaagtgagaatatttcatagaaatcctattgttttgatgtcttctaaaaaagaaaaaaagcaatgatctgtaacattttttaacttaaataattagattgatttaagtgacatcaaaacatctggaaaatggtgtggacacaaattcactagagagccatattttttgctaactaattgagaaattaatcactggcaagtctttggtaaaagtatcacctcagtcatgatctctcctgccttcatgacattttcctcattggtgtgaggatgctattctgctttctatgtgaccaggaaatagtgctgtcttctgtctagttatgatttaggttgtacaccaggttttcacatatgttccctaacgtctgtagtaggaccagggactggttggcttcaagttgttggatatggttaccttaagtcattcatgtacaggaactcatttgagatgataggaaatgaagtgaaagattttcttgcccctgttaagtaagataaaaaggattgttatgatggggcaggagcagatctatttccaataaacagaatttgaagtgtttgtgtgatattcagatacctcattgtcatttgaatgaattactcctgctctcagtgaagatgtctaagctgcaaataagaaatggagagcgctgtcagaagtcagatggaattgagaataggggcctggctgcaatctgtggagactgcctaaagcagctagataagaaactagcagctggggagagaaagatcgaatttagtcggcctgttttatattttcttataaaaaataactgcttcgaaatgtttgagaagatagaggcaatgagcagaaagttgttccttaaatcagttatagaatgaacacatacacgggcactcagatcaagccatgctgagcttgagacaccgggtgacgcgtgacttgtttattcccaggctgcaaaggagagtaaatgaagtaacgggaaggcccggtgtggtaggcacactcctgcctggcaccatctgctgcttttgtccctgttactccttgttcctttccctccttttctccctcccttcctccctccctctctccctccttcacacttctgtctttatttcctcctgggagttaattggtggtagcccctctgtgctgttctttcgggggtgcctttaatttcgacaatacaatgccatccatgggggcattttatatacagtaataattgtcattgatgtggccataaggtacttttttgtggtacccttcttgaacagaacagacacagaagggcgtgcgtgcgtgcgtgcgtgcgtgcgtgcgtgcgtgtgtgcgtgtgtgcgtgcgtgtgtgcgtgtgtgcgtgcgtgcgtgtgtgcgtgcgtgcgtgtgtgcgtgtgtgtgtgtgtgtgtgtgtgtgtgtgtgtgtgtgtgtgtgtgttgggatggggtggggagcgctagcttcctacttgttgtagggtgatgaggttttatatagtctgtttctgagacagttaccaaatccagctgggttactttttttttggttttttatgagacagggtttctctgtattgttttggaggctgtcggtccagcctggtctcgaactcacagagatccgcctgcctctgcctcccgagtgctgggattaaaggtgtgcgccaccaccgcccggccccagctgggttacttatcactcagtggatctttctcttttctttgtaagaagaactttgcattgtgggtcgtcatggaagaacacttggaaaggtaccctttctgccccacccgtttattgaatgagtcttttttttttttaattaaatagcagaactttggggaaagatttagaaaaggcccttttcatattataatacgaggtataggatggtttaagataagagactttttgttagctgttatcagttgagaaaggcacgag

SEQ ID NO: 8 - DNA, 2287 bp, Babesia microti
ttataaacatatctaaatattttaataataatgatgaaatttaacatagataagataatattaatcaatttaatagtattattgaatcgaaatgtagtgtattgtgtggatacaaataatagttcattaattgaatcacaaccagtaacaactaacattgacactgataatacaattacaacaaataaatacactggtactataattaatgccaatattgttgagtaccgtgaatttgaggatgaacctttaacaatagggtttagatacactatagataaatcacaacaaaataaattatcacatccaaataaaattgataaaatcaaattttctgattatataattgaatttgatgacaatgctaaattaccaactgataatgttatttgtatatccatctatacttgcaagcataataatccagtattaattagattctcatgttctatagaaaaatattactaccattacttctactcaatgaataatgatacaaataaatggaataatcacaaattaaaatatgataaaacatacaatgaatatactgacaataatggtgttaattattataaaatctattatagtgataaacagaattcccctactaatggaaatgaatatgaggatgtagcattagcaagaatacattgtaatgaagaaagatgtgcaaatgtaaaggtagataaaattaaatataagaatttggaaatttatgtgaaacagttaggtactataattaatgccaatattgttgagtaccttgtatttgaggatgaacctttaacaatagggtttagatacactatagataaatcacaacaaaatgaattatcacatccaaataaaatttataaaatcaaattttctgattatataattgaatttgatgatgatgctaaattaacaacaattggtactgttgaagatataaccatctatacttgcaagcataataatccagtattaattagattctcatgttctatagaaaaatattactactattacttctactcaatgaataataatacaaataaatggaataatcacaacttaaaatatgataatagattcaaagaacatagtgacaagaatggtattaattattatgaaatctcagctttcaaatggagtttctcttgttttttcgttaataaatatgagcataaagaattagcaagaatacattgtaatgaagaaagatgtgcaaatgtaaaggtagataaaattaaatataagaatttggaaatttatgtgaaacagttaggtactataattaatgccaatattgttgagtaccttgtatttgaggatgaacctttaacaatagggtttagatacactatagataaatcacaacaaaatgaattatcacatccaaataaaatttataaaatcaaattttctgattatataattgaatttgatgatgatgctaaattaacaacaattggtactgttgaagatataaccatctatacttgcaagcataataatccagtattaattagattctcatgttctatagaaaaatattactactattacttctactcaatgaataataatacaaataaatggaataatcacaacttaaaatatgataatagattcaaagaacatagtgacaagaatggtattaattattatgaaatctcagctttcaaatggagtttctcttgttttttcgttaataaatatgagcataaagaattagcaagaatacattgtaatgaagaaaaatgtgtaaatgtaaaggtagataacattgggaataaaaatttggaaatttatgtgaaataatttaatgaagtataatattatttataataattcaaagattaatataattaattattataattacaaaaataattaattgtagaatattatattattaatcaattcagattataaatacatatttttacatacatttcaatttaaacattcaaattaatgtcatttttatctacattattataattataactataatattcattaaatactatttaaaaaaatatcctctacattatatcaatcaatataatatacaattatataatatattcacaatgtataacaatcaaccctaacatgtacatacataatatcattactaatcaatatttaattaataaaatatttaatagtcatctgtaatataatcattgtatactaatttattataaattattacaaaatacactcttttacttcattttatttctgttaaatttcatattctaatattatattcatctttctcatgttactt

SEQ ID NO: 9 - DNA, 2784 bp, Babesia microti
cactgctttcgcagcgtttcttgcttttgggaatatctcacctgtactttctgctggtggtagtggtggtaatggtggtaatggtggtggtcatcaagagcaaaataatgctaatgatagtagtaatcccaccggagccggtggacaacccaataacgaaagtaagaaaaaggcagtaaaacttgacttggacctcatgaaagaaacaaagaatgtttgcaccactgttaatactaaactagtcggaaaagcaaagagcaaattaaacaaattagaaggtgaatcccataaggagtatgtagctgagaaaacgaaggagatagatgagaaaaataagaaatttaacgagaatcttgttaaaatagagaaaaagaagaaaattaaggttcctgccgatactggtgctgaagtggatgctgttgatgatggtgttgcgggtgcactatccgatttatcctccgatatctccgctattaagactctcaccgacgatgtatccgagaaggtttctgaaaacttgaaagatgatgaggccagtgcaacagaacacactgatataaaagaaaaagccaccctgcttcaagagtcttgcaacggaattggcactatcctagataagttggccgaatatttaaataatgatacaactcaaaatatcaagaaagaatttgatgaacgcaagaagaatctcacctctttgaagacaaaggtagaaaataaggatgaagattatgttgatgttaccatgacatcaaaaacagatctgataatacactgtttaacttgcacaaacgatgcacacggactgtttgatttcgaatcgaagagcttgataaaacaaacctttaaattgaggtccaaagatgaaggtgaactctgctaatttagattttagatgggccatgtatatgttaaacagcaagattcatcttatagaaagcagtttgatcgataacttcaccttggataatccatccgcatacgaaattttacgcgtttcttataactcaaatgaatttcaagtacaatcaccgcagaacattaacaatgaaatggaatcttcaacgcccgaatccaatatcatttgggttgtacatagtgatgttataatgaaaaggttcaactgtaaaaatcgcaaatctctcagtactcattcactcactgaaaatgatattctcaagtttggccgtatagaactctctgttaaatgtataattatgggcgcaggtatcactgcatctgatcttaatctaaagggattggggtttattagtccagataaacaatcaactaatgtatgtaactattttgaagatatgcatgaatcttatcatattcttgatacacaaagggcctcggattgtgtatcagatgatggcgctgatattgatatatccaacttcgacatggtccaagacggtaacataaattctgttgacgctgattctgaaacatgtatggcaaactctggcgtaacggtcaataatactgaaaatgttagtaatagtgagaattttggaaaattaaaatcattggtaagcaccaccactcctttgtgccgtatttgcctgtgtggtgaatcagaccctgggccactagtaaccccttgcaattgcaaggggtccctaaattatgtccatcttgaatgcctaaggacttggattaaagggcggttgtcaattgtgaaggatgatgatgcttcctttttctggaaagagctatcatgtgagctatgcgggaagccgtatccatcggtcctacaagtagatgatacagagactaatttgatggatataaaaaaaccggatgcaccatatgtggtattggaaatgagatcaaattctggtgatgggtgtttcgttgtttctgtagctaaaaataaggcgattattggacgggggcatgaaagtgacgttaggttgagtgatatttcagtgtcacgaatgcatgcttctttggaattggatggtggaaaagtagtgatacatgaccagcaatctaagtttggtacactcgttagggccaaagcgcctttttcaatgcctataaagggtcccatctgtctacaggtaagcattttctttttgaacttgaaaatatctactcatagtctaaccatggagaggggcatggaacatgtccttctctaatatttccaaaaaggatctatgcctgataaccttggtattgaaggtggctttctcaaagtgagacattccatttctgttgttggagctatcctatctgaggttagtgttctggtaaacattcctagaaaactcataaagcagaaatctgtgtgtatactaaattgcacagagaactccacgtgtgtgctagacttcacagagaactctgtgtgtgtgctaaactgcatagagaagaacatgttgagtgcatcatggttgagggaaattgctttatataaaagatttattttcctaaggtaacttaggattaatttttctgaaagcttagttttggtgagcacaattgtgatctttgtttctcagatggtcgggaaggcactcccagaaagcaggtggatacacactacactgcatgctacactctgtagactaggagtatcgttttcacacttatgaaatagtcaccatgctgggcacaaatatctttttatacaccatatattgttcatgttcaggtccacatttcaatttgtatgtgaaaagcatccggggctgtctgataaacacatagaaatgaaggaaacagtgtatgtaactgaagccttcagtcctttgcaatttctttgattcttag

SEQ ID NO: 10 - DNA, 3701 bp, Babesia microti
acctatttataatatagtatattactggtttgttttaaatcgaaaaaatgtattgtatttaagaatgaaattatttatttatcatgattatcatatttctaaatattaaaatctagtaacggttgcttgaatatttatttaaattatatgtagtagtattaaaatgtgttatatataagtagtgttctaaatcatcattagtaatattgtataaattaattgtaaaaattgcgatactacaattaatcaacaattaaaatatatcagtatagataatttaaataaataattagataagatcttaaggattaaatgacgaatttagaatgataaataatcatcataggcatttgttataatatcattaattatattcatgtggttataattataaaagtatatatagttttgtaattgtaatgatataaaattagaacagatataattaataattcaaatattatattaattttattatatatgattattattgatatttatataattacatattgttattgtatcatttaatgattatatatcaatatccatatatatatataataattgaattataattaaattaattggcatattacatttataataatatattattagtcaatatgacatcatattatattatccatcatgattgtgaatgtaactagaacattgattattatattaaatcacatattaatactgattataataatatcattgataatctaataatatagtattatctctaataatattgtattatctctaatattatggtataatagatactgtgaaaataaattcaactggagataaggaaaccattttgtatagatattttatacaaattattatgaaataatctaaataaatgacaaaaaatcgattatacaaatcacattaatgacaaacaaacttgtatacatatattgattaacattacaaaactaaattataatatttagattgataattgttataatacttaacaatattctactttttaatataattttttattcaataatatactctttcatattttgtactattttatataatcatatatattatataattatatatatttgataattgaatatatcaataatgatgatatacatgaatatgcatatataccccatataatgttattatatttagtgcttacattattaattataaatatatttaaataattaaataataatgaaaattaacatagacaatataatattaatcaatttgataatattattgaatcgtaatgtagtatattgtgtggataaaaatgatgtttcattatggaaatcaaaacctataacaactgtcagtaccactaatgatactattacaaataaatacactagtactgtaattaatgccaattttgctagctaccgtgaatttgaggatagggaacctttaacaataggatttgaatacatgatcgataaatcacaacaagataaattatcacatccaaataaaattgataaaatcaaaatttctgattatataattgaatttgatgacaatgctaaattaccaactggtagtgttaatgatatatccatcattacttgcaagcataataatccagtattaattagattctcatgtttaatagaaggatctatctgctattatttctacttattgaataatgatacaaataaatggaataatcacaaattaaaatatgataaaacatacaatgaacatactgacaataatggtattaattattataaaatcgattatagtgaatctacagaacctactaccgaatctactacctgtttttgttttcgcaaaaaaaatcataaatctgagcgtaaagaattagaaaattataaatatgagggtacagaattagcaagaatacattgtaataaagggaaatgtgtaaaattgggtgacattaagataaaggataagaatttggaaatttatgtgaaacagttaatgtctgtaaatactccagtaaattttgacaaccctacatcgattaatctaccaactgtcagtactaccaatgatactattacaaataaatacactggtactataattaatgccaatattgttgagtactgtgaatttgaggatgaacctttaacaatagggtttagatacactatagataaatcacaacaaaataaattatcacatccaaataaaattgataaaatcaaattttttgattatataattgaatttgatgatgatgttaaattaccaacaattggtactgtcaatattatatatatctatacttgcgagcataataatccagtattagttgaatttatagtttctatagaagaatcttactacttttacttctactcaatgaataataatacaaataaatggaataatcacaaattaaaatatgataaaagattcaaaaaatatactaagaatggtattaattgttatgaatatgtacttcgtaaatgcagttcttatactcgtaaaaatgaatatgagcataaagaattagcaagaatacattgtaatgaagaaaaatgtgtaaatgtaaaggtagataacattgagaaaaagaatttggaaatttatgtaaaataatttaacgaagtgtaatatgtaaaatagtttaatgaagtataatattatttaaaataattcaaaatttcagaaattaatataattaattattataaatacaaaataattaattacaaatgtgtattgttagttatttcagattgtaaatacatattttacatacatttttattaaaactttcaaattaatattttcatttttataagcattattataattatatactataattatcagtcatcaaataatatccaaagttatcctctacattatatcaatcatacagtatacaattatataaaatattaacaacatataacaaccaacattaatatatacataatatctttattaatcaatatttaatcaatacaataattaatagttaactaactatacacatagtgtatactaaattattataaattatatgttataattacaaaaacgtcatttacttattttatttcagttatgtttcatagtctaatttagatttggtgaaacgcatctggctgatgtgctggtgagcaagcagttccacgaagcaaacaatatgactgatgcgctggcggcgctttctgcggcggttgccgcacagctgccttgccgtgacgcgctgatgcaggagtacgacgacaagtggcatcagaacggtctggtgatggataaatggtttatcctgcaagccaccagcccggcggcgaatgtgctggagacggtgcgcggcctgttgcagcatcgctcatttaccatgagcaaccccgaaccgtattcgttcgttgattggcgcgtttgcgggcagcaatccggcagcgttccatgccgaagatggcagcggttacctgttcctggtggaaatgcttaccgacctcaacagccgtaacccgcaggtggcttcacgtctgattgaaccgctgattcgcctgaaacgttacgatgccaaacgtcaggagaaaatgcgcgcggcgctggaacagttgaaagggctggaaaatctctctggcgatctgtacgagaagataactaaagcactggcttgataaataaccgaatggcggcaatagcgccgccattcggggaatttacccctgttttct

SEQ ID NO: 11 - DNA, 1287 bp, Babesia microti
ctcgtgccgctcgtgccgattattataaatatttagttgatgaatatagttctcccagggaggaaagagaattagcaagagtacattgtaatgaagaaaaatgtgtaaaattggatggcattaagtttaaggataagaatttggaaatttatgtgaaacagttaatgtctgtaaatactccagttgtatttgacaacaatacattgattaatccaactagcagcagtggtgccactgatgacataacatatgaattatcggtggaatcacaacctgtaccaactaacattgacacaggtaataatattacaacaaatacatcaaataataatctaattaaagctaaatttctttataattttaatcttcctggtaaaccttcaacaggactatttgaatacactatagataaatcagaacaaaataaattatcacatccaaataaaattgataaaatcaaattttctgattatataattgaatttgatgatgatgctaaattaccaacaattggtactgtcaatattatatccatcattacttgcaagcataataatccagtattagttgaatttatagtttctacagaaatatattgctactacaattacttctactcaatgaataataatacaaataaatggaataatcacaaattaaaatatgataaaagatataaagaagaatatacagatgataatggtattaattattataaattaaatgatagtgaacctactgaatctacagaatctactacctgtttttgttttcgcaaaaaaaatcataaatatgaaaatgagcgtacagcattagcaaaagaacattgcaatgaagaaagatgtgtaaaggtagataacattaaggataataatttggaaatttatctaaaataatttaacgaagtataatattatttataataattcaaaatttcagaaattaatataattaattattataaatacaaaataattaattacaaatgtgtattgttagttatttcagattgtaaatacatattttacatacatttttattaaaactttcaaattaatattttcatttttataagcattattataattatatactataattatcagtcatcaaataatatccaaagttatcctctacattatatcaatcatacagtatacaattatataaaatattaacaacatataacaaccaacattaatatatacataatatctttattaatcaatatttaatcaatacaataattaatagttaactaactatacacatagtgtatactaaatt

SEQ ID NO: 12 - DNA, 572 bp, Babesia microti
cttcattgacgtctatccccaatcttagaaaaatcttcaaatcgattctagaataactggaaacaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaagtgataccgatcccgaagctggtgggcctagtgaagctggtgggcctagtgaagctggtgggcctagtggaactgttgggcccagtgaagctggtgggcctagtgaagctggtgggcctagtggaactggttggcctagtgaagctggtgggcctagtgaagctggtgggcctagtggaactggttggcctagtgaagctggttggtctagtgaacgatttggatatcagcttcttccgtattctagaagaatagttacatttaatgaagtttgtttat

SEQ ID NO: 13 - DNA, 2338 bp, Babesia microti
ctcgtgccgaatcttagaaaaatcttcaaatcgattctagaataactggaaacaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaagtgataccgatcccgaagctggtgggcctagtggaactgttgggcccagtgaagctggtgggcctagtgaagctggtgggcctagtggaactggttggcctagtgaagctggtgggcctagtgaagctggtgggcctagtggaactggttggcctagtgaagctggttggtctagtgaacgatttggatatcagcttcttccgtattctagaagaatagttacatttaatgaagtttgtttatcttatatatacaaacatagtgttatgatattggaacgagatagggtgaacgatggtcataaagactacattgaagaaaaaaccaaggagaagaataaattgaaaaaagaattggaaaaatgttttcctgaacaatattcccttatgaagaaagaagaattggctagaatatttgataatgcatccactatctcttcaaaatataagttattggttgatgaaatatcaaacaaggcctatggtacattggaaggtccagctgctgataattttgaccatttccgtaatatatggaagtctattgtacttaaagatatgtttatatattgtgacttattattacaacatttaatctataaattctattatgacaataccattaatgatatcaagaaaaattttgacgaatccaaatctaaagctttagttttgagggataagatcactaaaaaggacgtgtatgtaaatgatcactaaacgggctccacatatctattactggggtagatattataagttatggataagtaaatttatggcgatagattccaacaaatttgtggttagtagcgacaatgattatggctagtgtgtggagtacttatgagtgaatgattgtagtggtggctagcagtgagtatagttaggtaatccctacacacccatttaaataagatgcaaatagcatttaaattgacatatattgtgtgtatgtccacgtttattgcgtttccatgacgtatctgctgaggtgtgtcttgtgtatctaagtaccagacacagcacttaaattgttatgggcatgacgatggatgttaaaggtttatacactccaaaggcacgttcttctgctagggaaacgagggacaagttcgattttgctatacaaagcaagtttcactccctggactttacactggatgactttgatataggtgcattcgtggtaaacctcaaaatttactcagggcgatggtgcccatgggcaggtttttttggcaagggaacgacgtaccggttttatttgcgtgttaaaatgcatttttaaatcacaacttgtgaagtaattgcctaataatcacacagaaatggacaggaagctattttcaagcgggaaatcgaattgcacgggcatctgagacatccaaacatagcatggtatgtacatatttatccagcttgtatacctggttcactagccctactatgatattcatagtgatggaatattgttacaatggcgatctatttaattatatgtcaaaacatggccaactgagtgaagaaagggtatcagagtatacagatatttacatagaattttgttcgaagtcatttgggccattagaagctgccacgacaaacgcatagcgcacttggatattaaaccagtaaggttctatgttacagaggagaatatattattggaccatgaaaacaggtgtaaattggcggactttggattctctgcacacatagggcatttgtaccgctcaaacggagtgctcatcatcgtggcacgcatggtaacacgcaattwatggcagattattggtctccggagcagtgtgccaaacatttgggtctggggttgaagtatggggagtatgatgaacaaagcgacatatgggcgttgggcatattggcagttgaattgtttattggataccctccatttggatctactactgaagagcccaacaatgtgattatgaacagaatccacacttaccactggaccaaacatgtacttttatctattacgcagatttttgaaatgaagagggaaaaacatctactctcgtcgacgcctg

SEQ ID NO: 14 - DNA, 729 bp, Babesia microti
ttgcctggaccttctctgtcctagaattacaggaattctcttatactgtttaatacaaaacacttggaagaatttcaccaattgcatatgaaacatggaatccaagagaccaaaatttaaaaccttgaaatagaagcacttatgccaatattggaaattacttagtgaagtgatccaaagtactgatttggtcagaagacatcaccagggcactagctggcctagtgacctgagtatttgtgaaagctgattttaatgttgagaacatgaaggaagcagtattgaggtaatggaatcttgtagattatagtagaagccaactgagaccaagaaatgtacggtaggaatgaaataaggtcttgggtggtcattgcatggagctgtgaaagtgaagcgttgttggggtatagattcgcaagtcttggggcatgactatgtggggttaccaaggttaggttaactgaggtggaaagatccactctaaatgggggagttaccatttcatgtgctgggatcccagagatgtcaaaggagaaaataagctattgaataagagcatctatatcccttgcttcttggctatggatgttatgtgactagtcatctcttagtcttaccttcaccattataacaagattttctagaactttgggttaaattaaatcctttattcctcacgttgctgtcttagttactttcctgttgctttgataaagcattctggccaag

SEQ ID NO: 15 - DNA, 1448 bp, Babesia microti
acatgttgacttttggaaatatacgttttcataatataaatctcccaccattttcattgggcataattcactcgattacggtagaaaaggcgattaactctgaagattttgacggaatacaaacacttttacaagtgtctatcattgctagttacggtccatctggcgattacagtagttttgtgttcactccagttgtaacagcagacaccaacgttttttacaaattagagacggatttcaaacttgatgttgatgttattactaagacatcactagaattgcccacaagtgttcctggctttcactacaccgaaactatttaccaaggcacagaattgtcaaaatttagcaagcctcagtgcaaacttaacgatcctcctattacaacaggatcggggttgcaaataatacatgatggtttgaataattcgacaattataaccaacaaagaagttaatgtggatggaacagatttagttttttttgaattgctccctccatcggatggcattcccaccttgcgatcaaaattatttcccgtcctgaaatcaattccaatgatatctaccggggttaatgaattactgttggaagtactcgagaacccctctttccctagtgcaattagcaattacaccggactgacaggccgacttaacaaattacttacagttttagacggtattgttgatagcgccattagtgtcaagactacagaaactgtccctgacgacgcagaaacttctatttcttcattgaaatcattgataaaggcaatacgagataatattactaccactcgaaacgaagttaccaaagatgatgtttatgcattgaagaaggccctcacttgtctaacgacacacctaatatatcattcaaaagtagatggtatatcattcgacatgctgggaacacaaaaaaataaatctagcccactaggcaagatcggaacgtctatggacgatattatagccatgttttcgaatcccaatatgtatcttgtgaaggtggcgtacttgcaagccattgaacacatttttctcatatcaaccaaatacaatgatatatttgattacaccattgattttagtaagcgtgaagctactgattctggatcatttaccgatatattgctcggaaacaaggtgaaggaatctttgtcatttattgagggtttgatttctgacataaaatctcactcattgaaagctggggttacaggaggtatatcaagttcatcattatttgatgaaatcttcgacgagttaaatttggatcaagcaacaattagaacccttgttgcaccattagattggccacttatctcagacaaaagcctccacccttcactgaagatggttgtggtcctgccaggatttttcatagttccttaataacatgacatttcatagtcccttcagtcctgatgacaagacggtgaa

SEQ ID NO: 16 - DNA, 1350 bp, Babesia microti
gcctaagcccaaatgggatttaagcaggaggggataaaacagatgacctccaccatgccctactaactctaagctaaggaaatccagcctgctggctatttacctgctttcctcgaagtgaaaggccagagtcacccccaatctttcccaaaagattgaagtcactctctccatgccggcaaaggtagatggtgcgaggctggacatggatattcataaggtagtagacaattttactctggatgtagtcctggactctgttgaccagaaatctctggcctacattaatcaccttgatgaagacagatccctaggacagagtagaaagagcaattttatggtcagaaaatctgaaactaggagtgtggcaagcaagggggcaaggctatcagcacctagtgacaatcccagcacttagaaggcttagctggaaggggcttaggtttgaccctgactcaagacaaatgaacatatgaaaagtatggggagaatgatctgtgtattgactggtagggcctcatcagctattccttctctccctgtcactgccatctcgtgccgaattcggcacgagctcgtgccgaaaccctaaaccctaaacccctaaaccctaaaccctaaaccctaaaccctaaaccctaaaccctaaaccctaaaccctaaacccctaaacccctaaaccctaaaccctaaaccctaaaccctaaaccctaaaccctaaaccctaaccctaaccctaaccctaaccctaacctagccttcattgacgtctatccccaatcttagaagaatcttcaaatcgattctagaataactggaaacaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaagtgataccgatcccgaagctggtgggcctagtgaagctggtgggcctagtggaactgttgggcccagtgaagctggtgggcctagtgaagctggtgggcctagtggaactggttggcctagtgaagctggtgggcctagtgaagctggtgggcctagtgaagctggtgggcctagtgaagctggtgggcctagtggaactggttggcctagtggaactggttggcctagtgaagctggttggtctagtgaacgatttggatatcagcttcttccgtattctagaagaatagttatattt

SEQ ID NO: 17 - DNA, 1820 bp, Babesia microti
ggaaagccttaaacatgcatgggaataatgaaatagtaaaaattgcagccatggcaatgtaataatgagtggatgtttcagtcttgaggctctttaacaagagtgttgtcttgtagtcaaagacaaagtgattcgtcatgccgcattcgcagccaccatcatcatcaggcgacgacgggtctctttcattatcctcgggcttattattgcaaccatgacacccttctttacaaaagtctttttttttcagcggtgtctgagtattatgcgattttattccagccttcccacttttattcttattgagattgccatgctcttcttcatgagcgtcacttgtttcctgcggtgtctgagtatcatacgattttattccagcatttccacttttattcttattgattttgtcatgcccttcttcacactcttcacatatttcttgcgttgtctgagtatcatgcgattttctttcagccttctcacttttattcgtattgattttgtcatgcccttcttcatgagcgtcacttgtttcctgcggtgtctgagtatcatacgattttattccagcatttccacttttattcttattgattttgtcatgcccttcttcacactcttcacatatttcttgcgttgtctgagtatcatacgattttattccagcatttccacttttattcttattgattttgtcatgcccttcttcacactcttcacatatttcttgcgttgtctgagtatcatgcgattttctttcagccttctcacttttattcgtattgggtttgccatgcccttctttacgctcttcatatatttcttgtgccgttagtctcagtaagttgtcaagctcttcatatatttcttgcggtgtctgagtatcatgcgattttctttcagtcttctcacttttattcgtattgagtttgccattcccttcttcatgatcgtcacttgtttcttgcgccgttagtctcattaagttgtcaagctcttcatcatctattgaatggtatggagctgtatcttcccagggtggttgaattatgtcattctcgccgattttaaatgatggttcttcatcatttatatcagatgccatgtctgagtggtgccctaatctagagaattggtgtggtaccccctcatccaaactttcgggcaacaccctggtatcagaatccatttgttcgagcggctcactatcgcaagcgtcttgtggattgatgttatcatgttcctggatttcaacatgtacagattctgaatccgcattgggttctggaatatagttggtaactacatttgtttctagagaagtatcattcttatattaattcatctaagatctgtgcttctttgtttctacacatacagggtgtctcttttcccaacataatatctgtaaattcttcccagaagcagaaccttgttggtaccagacagcatcgggtctctgtgagtttctattcaggcaacaggtgtattctgtttgccagtccaagtgcatcctgtattctagtactggcttactaccccaagcaaatcactggcatcaacatctagcactgagtgaagcatgatctcttctacaaggtgtttttccattgtgttgtaagcccgtatacaaggctgttcccactcaacaatgaagagacctcttagcatgaatggccagatgtctgttctttaaattaaatcaatatgttttgctcaatatgtcagacttgtttgtggtggagccaaaattggaggtcccatcgagatttggagaaacttgaaatgaatgcaaaagatggtgggggctactcgtgccg

SEQ ID NO: 18 - protein, 263 aa, Babesia microti
LFLMSGAVFASDTDPEAGGPSEAGGPSGTVGPSEAGGPSEAGGPSGTGWPSEAGGPSEAGGPSEAGGPSEAGGPSGTGWPSGTGWPSEAGWSSERFGYQLLPYSRRIVIFNEVCLSYIYKHSVMILERDRVNDGHKDYIEEKTKEKNKLKKELEKCFPEQYSLMKKEELARIFDNASTISSKYKLLVDEISNKAYGTLEGPAADNFDHFRNIWKSIVLKDMFIYCDLLLQHLIYKFYYDNTVNDIKKNFDESKSKALVLRDKI

SEQ ID NO: 19 - protein, 310 aa, Babesia microti
MSGAVFASDTDPEAGGPSEAGGPSGTVGPSEAGGPSEAGGPSGTVGPSEAGGPSEAGGPSGTGWPSEAGGPSEAGGPSGTVGPSEAGGPSEAGGPSGTGWPSEAGGPSEAGGPSEAGGPSEAGGPSGTGWPSGTGWPSEAGWSSERFGYQLLPYSRRIVIFNEVCLSYIYKHSVMILERDRVNDGHKDYIEEKTKEKNKLKKELEKCFPEQYSLMKKEELARIFDNASTISSKYKLLVDEISNKAYGTLEGPAADNFDHFRNIWKSIVLKDMFIYCDLLLQHLIYKFYYDNTVNDIKKNFDESWTQTLKE

SEQ ID NO: 20 - protein, 367 aa, Babesia microti
LWFIKMVSFKSILVPYITLFLMSGAVFASDTDPEAGGPSEAGGPSGTVGPSEAGGPSEAGGPSGTGWPSEAGGPSEAGGPSEAGGPSEAGGPSGTGWPSGTGWPSEAGWSSERFGYQLLPYSRRIVIFNEVCLSYIYKHSVMILERDRVNDGHKDYIEEKTKEKNKLKKELEKCFPEQYSLMKKEELARIFDNASTISSKYKLLVDEISNKAYGTLEGPAADNFDHFRNIWKSIVLKDMFIYCDLLLQHLIYKFYYDNTVNDIKKNFDESKSKALVLRDKITKKDGDYNTHFEDMIKELNSAAEEFNKIVDIMISNIGDYDEYDSIASFKPFLSMITEITKITKVSNVIIPGIKALTLTVFLIFITK

SEQ ID NO: 21 - protein, 492 aa, Babesia microti
MYKIKISDYIIEFDDNAKLPTDNVIGISIYTCEHNNPVLIEFYVSKKGSICYYFYSMNNDTNKWNNHKIKYDKRFNEHTDMNGIHYYYIDGSLLASGEVTSNFRYISKEYEYEHTELAKEHCKKEKCVNVDNIEDNNLKIYAKQFKSVVTTPADVAGVSDGFFIRGQNLGAVGSVNEQPNTVGMSLEQFIKNELYSFSNEIYHTISSQISNSFLIMMSDAIVKHDNYILKKEGEGCEQIYNYEEFIEKLRGARSEGNNMFQEALIRFRNASSEEMVNAASYLSAALFRYKEFDDELFKKANDNFGRDDGYDFDYINTKKELVILASVLDGLDLIMERLIENFSDVNNTDDIKKAFDECKSNAIILKKKILDNDEDYKINFREMVNEVTCANTKFEALNDLIISDCEKKGIKINRDVISSYKLLLSTITYIVGAGVEAVTVSVSATSNGTESGGAGSGTGTSVSATSTLTGNGGTESGGTAGTTTSSGTWFGK

SEQ ID NO: 22 - protein, 138 aa, Babesia microti
SLGQPASLGQPASLGQPASLGQPASLGQPASLGQPVPLGPPASLGPPASLGPPASLGQPVPLGPPASLGPPASLGPPASLGPPASLGPPASLGPPASLGPPASLGPPASLGPTVPLGPPASRSVSPAKTAPLIKKSVI

SEQ ID NO: 23 - protein, 303 aa, Babesia microti
LWFIKMVSFKSILVPYITLFLMSGAVFAGDTDREAGGPSGTVGPSEAGGPSEAGGPSEAGGPSEAGGPSEAGGPSEAGGPSEAGGPSEAGGPSGTGWPSEAGWPSEAGWPSEAGWPSEAGWPSEAGWPSERFGYQLLWYSRRIVIFNEIYLSHIYEHSVMILERDRVNDGHKDYIEEKTKEKNKLKKELEKCFPEQYSLMKKEELARIIDNASTISSKYKLLVDEISNKAYGTLEGPAADDFDHFRNIWKSIVPKNMFLYCDLLLKHLIRKFYCDNTINDIKKNFDDIEKLGCFQARSFLPVN

SEQ ID NO: 24 - protein, 592 aa, Babesia microti
MMKFNIDKIILINLIVLLNRNVVYCVDTNNSSLIESQPVTTNIDTDNTITTNKYTGTIINANIVEYREFEDEPLTIGFRYTIDKSQQNKLSHPNKIDKIKFSDYIIEFDDNAKLPTDNVICISIYTCKHNNPVLIRFSCSIEKYYYHYFYSMNNDTNKWNNHKLKYDKTYNEYTDNNGVNYYKIYYSDKQNSPTNGNEYEDVALARIHCNEERCANVKVDKIKYKNLEIYVKQLGTIINANIVEYLVFEDEPLTIGFRYTIDKSQQNELSHPNKIYKIKFSDYIIEFDDDAKLTTIGTVEDITIYTCKHNNPVLIRFSCSIEKYYYYYFYSMNNNTNKWNNHNLKYDNRFKEHSDKNGINYYEISAFKWSFSCFFVNKYEHKELARIHCNEERCANVKVDKIKYKNLEIYVKQLGTIINANIVEYLVFEDEPLTIGFRYTIDKSQQNELSHPNKIYKIKFSDYIIEFDDDAKLTTIGTVEDITIYTCKHNNPVLIRFSCSIEKYYYYYFYSMNNNTNKWNNHNLKYDNRFKEHSDKNGINYYEISAFKWSFSCFFVNKYEHKELARIHCNEEKCVNVKVDNIGNKNLEIYVK

SEQ ID NO: 25 - protein, 463 aa, Babesia microti
IIMKINIDNIILINLIILLNRNVVYCVDKNDVSLWKSKPITTVSTTNDTITNKYTSTVINANFASYREFEDREPLTIGFEYMIDKSQQDKLSHPNKIDKIKISDYIIEFDDNAKLPTGSVNDISIITCKHNNPVLIRFSCLIEGSICYYFYLLNNDTNKWNNHKLKYDKTYNEHTDNNGINYYKIDYSESTEPTTESTTCFCFRKKNHKSERKELENYKYEGTELARIHCNKGKCVKLGDIKIKDKNLEIYVKQLMSVNTPVNFDNPTSINLPTVSTTNDTITNKYTGTIINANIVEYCEFEDEPLTIGFRYTIDKSQQNKLSHPNKIDKIKFFDYIIEFDDDVKLPTIGTVNIIYIYTCEHNNPVLVEFIVSIEESYYFYFYSMNNNTNKWNNHKLKYDKRFKKYTKNGINCYEYVLRKCSSYTRKNEYEHKELARIHCNEEKCVNVKVDNIEKKNLEIYVK

SEQ ID NO: 26 - protein, 297 aa, Babesia microti
RAARADYYKYLVDEYSSPREERELARVHCNEEKCVKLDGIKFKDKNLEIYVKQLMSVNTPVVFDNNTLINPTSSSGATDDITYELSVESQPVPTNIDTGNNITTNTSNNNLIKAKFLYNFNLPGKPSTGLFEYTIDKSEQNKLSHPNKIDKIKFSDYIIEFDDDAKLPTIGTVNIISIITCKHNNPVLVEFIVSTEIYCYYNYFYSMNNNTNKWNNHKLKYDKRYKEEYTDDNGINYYKLNDSEPTESTESTTCFCFRKKNHKYENERTALAKEHCNEERCVKVDNIKDNNLEIYLK

SEQ ID NO: 27 - protein, 121 aa, Babesia microti
LWFIKMVSFKSILVPYITLFLMSGAVFASDTDPEAGGPSEAGGPSEAGGPSGTVGPSEAGGPSEAGGPSGTGWPSEAGGPSEAGGPSGTGWPSEAGWSSERFGYQLLPYSRRIVTFNEVCL

SEQ ID NO: 28 - protein, 267 aa, Babesia microti
LWFIKMVSFKSILVPYITLFLMSGAVFASDTDPEAGGPSGTVGPSEAGGPSEAGGPSGTGWPSEAGGPSEAGGPSGTGWPSEAGWSSERFGYQLLPYSRRIVTFNEVCLSYIYKHSVMILERDRVNDGHKDYIEEKTKEKNKLKKELEKCFPEQYSLMKKEELARIFDNASTISSKYKLLVDEISNKAYGTLEGPAADNFDHFRNIWKSIVLKDMFIYCDLLLQHLIYKFYYDNTINDIKKNFDESKSKALVLRDKITKKDVYVNDH

SEQ ID NO: 29 - protein, 16 aa, Babesia microti
AWTFSVLELQEFSYTV

SEQ ID NO: 30 - protein, 465 aa, Babesia microti
MLTFGNIRFHNINLPPFSLGIIHSITVEKAINSEDFDGIQTLLQVSIIASYGPSGDYSSFVFTPVVTADTNVFYKLETDFKLDVDVITKTSLELPTSVPGFHYTETIYQGTELSKFSKPQCKLNDPPITTGSGLQIIHDGLNNSTIITNKEVNVDGTDLVFFELLPPSDGIPTLRSKLFPVLKSIPMISTGVNELLLEVLENPSFPSAISNYTGLTGRLNKLLTVLDGIVDSAISVKTTETVPDDAETSISSLKSLIKAIRDNITTTRNEVTKDDVYALKKALTCLTTHLIYHSKVDGISFDMLGTQKNKSSPLGKIGTSMDDIIAMFSNPNMYLVKVAYLQAIEHIFLISTKYNDIFDYTIDFSKREATDSGSFTDILLGNKVKESLSFIEGLISDIKSHSLKAGVTGGISSSSLFDEIFDELNLDQATIRTLVAPLDWPLISDKSLHPSLKMVVVLPGFFIVP

SEQ ID NO: 31 - protein, 128 aa, Babesia microti
LWFIKMVSFKSILVPYITLFLMSGAVFASDTDPEAGGPSEAGGPSGTVGPSEAGGPSEAGGPSGTGWPSEAGGPSEAGGPSEAGGPSEAGGPSGTGWPSGTGWPSEAGWSSERFGYQLLPYSRRIVIF

SEQ ID NO: 32 - protein, 245 aa, Babesia microti
QECCLVVKDKVIRHAAFAATIIIRRRRVSFIILGLIIATMTPFFTKVFFFQRCLSIMRFYSSLPTFILIEIAMLFFMSVTCFLRCLSIIRFYSSISTFILIDFVMPFFTLFTYFLRCLSIMRFSFSLLTFIRIDFVMPFFMSVTCFLRCLSIIRFYSSISTFILIDFVMPFFTLFTYFLRCLSIIRFYSSISTFILIDFVMPFFTLFTYFLRCLSIMRFSFSLLTFIRIGFAMPFFTLFIYFLCR

SEQ ID NO: 33 - protein, 293 aa, Babesia microti
TAFAAFLAFGNISPVLSAGGSGGNGGNGGGHQEQNNANDSSNPTGAGGQPNNESKKKAVKLDLDLMKETKNVCTTVNTKLVGKAKSKLNKLEGESHKEYVAEKTKEIDEKNKKFNENLVKIEKKKKIKVPADTGAEVDAVDDGVAGALSDLSSDISAIKTLTDDVSEKVSENLKDDEASATEHTDIKEKATLLQESCNGIGTILDKLAEYLNNDTTQNIKKEFDERKKNLTSLKTKVENKDEDYVDVTMTSKTDLIIHCLTCTNDAHGLFDFESKSLIKQTFKLRSKDEGELC

SEQ ID NO: 34 - protein, 431 aa, Babesia microti
GPKMKVNSANLDFRWAMYMLNSKIHLIESSLIDNFTLDNPSAYEILRVSYNSNEFQVQSPQNINNEMESSTPESNIIWVVHSDVIMKRFNCKNRKSLSTHSLTENDILKFGRIELSVKCIIMGAGITASDLNLKGLGFISPDKQSTNVCNYFEDMHESYHILDTQRASDCVSDDGADIDISNFDMVQDGNINSVDADSETCMANSGVTVNNTENVSNSENFGKLKSLVSTTTPLCRICLCGESDPGPLVTPCNCKGSLNYVHLECLRTWIKGRLSIVKDDDASFFWKELSCELCGKPYPSVLQVDDTETNLMDIKKPDAPYVVLEMRSNSGDGCFVVSVAKNKAIIGRGHESDVRLSDISVSRMHASLELDGGKVVIHDQQSKFGTLVRAKAPFSMPIKGPICLQVSIFFLNLKISTHSLTMERGMEHVLL

SEQ ID NO: 35 - protein, 6 aa, Babesia microti
  VARIANT (1)...(1): Xaa = Glutamic Acid or Glycine
XXXXXS

SEQ ID NO: 36 - protein, 32 aa, Babesia microti
  VARIANT (6)...(6): Xaa = Methionine or Isoleucine
RCLSIXRFXXSXXTFIXIXXXMXFFXXXXXFL

SEQ ID NO: 37 - DNA, 1820 bp, Babesia microti
cggcacgagtagcccccaccatcttttgcattcatttcaagtttctccaaatctcgatgggacctccaattttggctccaccacaaacaagtctgacatattgagcaaaacatattgatttaatttaaagaacagacatctggccattcatgctaagaggtctcttcattgttgagtgggaacagccttgtatacgggcttacaacacaatggaaaaacaccttgtagaagagatcatgcttcactcagtgctagatgttgatgccagtgatttgcttggggtagtaagccagtactagaatacaggatgcacttggactggcaaacagaatacacctgttgcctgaatagaaactcacagagacccgatgctgtctggtaccaacaaggttctgcttctgggaagaatttacagatattatgttgggaaaagagacaccctgtatgtgtagaaacaaagaagcacagatcttagatgaattaatataagaatgatacttctctagaaacaaatgtagttaccaactatattccagaacccaatgcggattcagaatctgtacatgttgaaatccaggaacatgataacatcaatccacaagacgcttgcgatagtgagccgctcgaacaaatggattctgataccagggtgttgcccgaaagtttggatgagggggtaccacaccaattctctagattagggcaccactcagacatggcatctgatataaatgatgaagaaccatcatttaaaatcggcgagaatgacataattcaaccaccctgggaagatacagctccataccattcaatagatgatgaagagcttgacaacttaatgagactaacggcgcaagaaacaagtgacgatcatgaagaagggaatggcaaactcaatacgaataaaagtgagaagactgaaagaaaatcgcatgatactcagacaccgcaagaaatatatgaagagcttgacaacttactgagactaacggcacaagaaatatatgaagagcgtaaagaagggcatggcaaacccaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagggcatgacaaaatcaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagagcatggcaatctcaataagaataaaagtgggaaggctggaataaaatcgcataatactcagacaccgctgaaaaaaaaagacttttgtaaagaagggtgtcatggttgcaataataagcccgaggataatgaaagagacccgtcgtcgcctgatgatgatggtggctgcgaatgcggcatgacgaatcactttgtctttgactacaagacaacactcttgttaaagagcctcaagactgaaacatccactcattattacattgccatggctgcaatttttactatttcattattcccatgcatgtttaaggctttcc

SEQ ID NO: 38 - protein, 445 aa, Babesia microti
YKNDTSLETNVVTNYIPEPNADSESVHVEIQEHDNINPQDACDSEPLEQMDSDTRVLPESLDEGVPHQFSRLGHHSDMASDINDEEPSFKIGENDIIQPPWEDTAPYHSIDDEELDNLMRLTAQETSDDHEEGNGKLNTNKSEKTERKSHDTQTPQEIYEELDNLLRLTAQEIYEERKEGHGKPNTNKSEKAERKSHDTQTTQEICEECEEGHDKINKNKSGNAGIKSYDTQTTQEICEECEEGHDKINKNKSGNAGIKSYDTQTPQETSDAHEEGHDKINTNKSEKAERKSHDTQTTQEICEECEEGHDKINKNKSGNAGIKSYDTQTPQETSDAHEEEHGNLNKNKSGKAGIKSHNTQTPLKKKDFCKEGCHGCNNKPEDNERDPSSPDDDGGCECGMTNHFVFDYKTTLLLKSLKTETSTHYYIAMAAIFTISLFPCMFKAF

SEQ ID NO: 39 - protein, 32 aa, Babesia microti
  VARIANT (3)...(3): Xaa = Glycine or Aspartic Acid
GHXKXNXNKSXXAXXKSXDTQTXQEXXXXXEE

SEQ ID NO: 40 - DNA, 2430 bp, Babesia microti
tgtattgtgtagataaaaatgatgtttcattatggaaatcaaaacctataacaactgtcagtaccactaatgatactattacaaatacacacactactaatgtaattaatgccaatcttattggccactttaattataaggatagggaacctttaacaatagtatttgtatacatgatcgatgaatcagaacaaaataaattatcacatccgaataaaattgataaaatcaaaatttctgattatataattgaatttgatgacaatgctaaattaccaactggtagtgttattgatttaaacatctatacttgcaaacataataatccagtattaattgaattttatgtttctatagaaggatctttctgctattatttctctcattgaataatgatacaaatgaatggaataatcacaaaataaaatatgataaaaaatataaagaatatacggacatgaatggtattcattattattatattgatggtagtttacttgtaagtggcgaagttacatctaattttcgttatatttctaaagaatatgaatatgagcatacaggattagtaaaaaaatattgtaatgaagaaagatgtgtaaaattggataacattaagataaaggataataatttggaaatttatgtgaaataatttaatgaagtataatattatttataataattcaaagattaatataatcaattattataattacaaaaataattaattgtagaatattatattattaatcaattcagattataaatacatatttttacatacatttcaatttaaacattcaaattaatgtcatttttatctacattattataattataactataatattcattaaatactattaaaaaaaatatcctctacattatattaattattatagtatgtcattatataacatattcacaacgtataacaaatcaatcattaacatatacatatatgatatcattaataatcaatatttaattgatacaataatcaatagtcatctgtaatataatcattgtatactaatttattataaattattacaaaatacactcttttacttcattttatttctgttaaatttcatattctaatattatattcatctttctcatgttactttaatctatttccatatttatcccaatttcttcatttaagactgagatgttcgttcgttcatacataaataatgtgtaaattttgtaatatataataatgtatacatctggtattacatctattttgtaataaatattaaaaaaacggttaaagttagtgccttaattccaggaattattacattagaaactttggtgattttagtgatttcggtgatcattgaaagaaatggtttgaaacttgcaatactgtcatactcatcataatccccaatgttggaaatcatgatgtcaacaattttattaaattcttctgctgcactattcaactccttaatcatgtcctcaaaatgagtgttataatctccatcctttttagtgatcttatccctcaaaactaaagctttagatttggattcgtcaaaatttttcttgatatcattaacggtattgtcataatagaatttatagattaaatgttgtaataataagtcacaatatataaacatatctttaagtacaatagacttccatatattacggaaatggtcaaaattatcagcagctggaccttccaatgtaccataggccttgtttgatatttcatcaaccaataacttatattttgaagagatagtggatgcattatcaaatattctagccaattcttctttcttcataagggaatattgttcaggaaaacatttttccaattcttttttcaatttattcttctccttggttttttcttcaatgtagtctttatgaccatcgttcaccctatctcgttccaatatcataacactatgtttgtatatataagataaacaaacttcattaaatataactattcttctagaatacggaagaagctgatatccaaatcgttcactagaccaaccagcttcactaggccaaccagttccactaggccaaccagttccactaggcccaccagcttcactaggcccaccagcttcactaggcccaccagcttcactaggcccaccagcttcactaggccaaccagttccactaggcccaccagcttcactaggcccaccagcttcactgggcccaacagttccactaggcccaccagcttcactaggcccaccagcttcgggatcggtatcacttgcaaagacagcaccgctcattaaaaagagtgtaatataaggaactaatattgatttaaatgacaccatctttataaaccatagttattggtacattattagtacattattggtatatgattggtacgtggtagtgattgtggtgctgcatctagtt

SEQ ID NO: 41 - protein, 128 aa, Babesia microti
YCVDKNDVSLWKSKPITTVSTTNDTITNTHTTNVINANLIGHFNYKDREPLTIVFVYMIDESEQNKLSHPNKIDKIKISDYIIEFDDNAKLPTGSVIDLNIYTCKHNNPVLIEFYVSIEGSFCYYFSH

SEQ ID NO: 42 - DNA, 1271 bp, Babesia microti
tgagaaaacgcatataattgtaactacgccagagaagtttgacgtagttacacgtaaaacaggcaatgagcccctgcttgagcggcttagattggttataattgatgaaatacacctactccatgacactaggggtccagtgctggaggctattgtggcccgcctgagtcagaggcccgaacgcgtaaggctagttggtctatcggccacgcttccaaactacgaagacgtggctagatttctcactgttaatctagaccgagggcttttctactttggcagccactttaggcctgtgcccttggagcaggtgtattatggcgtgaaggagaagaaggctatcaaacgtttcaacgcaatcaacgaaattctctaccaagaggtgattaacgatgtttctagctgccaaattcttgtttttgtgcattctagaaaggaaacgtacaggacggcaaaatttatcaaagacacggccctttcacgggacaacttgggagcctaaaccctaaaccctaaaccctaaaccctaaccctaaaccctaaaccctaaaccctaaaccctaaaccctaaccctaaccctaaccctaaccctaacctagccttcattgacgtctatccccaatcttagaaaaatcttcaaatcgattctagaataactggaagcaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactggttggcctagtgaagctggtgggcctagtgaagctggtgggcctagtgaagctggtgggcctagtggaactggttggcctagtgaagctggttggcctagtgaagctggttggcctagtgaagctggttggcctagtgaagctggttggcctagtgaagctggttggcctagtgaa

SEQ ID NO: 43 - protein, 166 aa, Babesia microti
EKTHIIVTTPEKFDVVTRKTGNEPLLERLRLVIIDEIHLLHDTRGPVLEAIVARLSQRPERVRLVGLSATLPNYEDVARFLTVNLDRGLFYFGSHFRPVPLEQVYYGVKEKKAIKRFNAINEILYQEVINDVSSCQILVFVHSRKETYRTAKFIKDTALSRDNLGA

SEQ ID NO: 44 - protein, 154 aa, Babesia microti
LWFIKMVSFKSILVPYITLFLMSGAVFAGDTDREAGGPSGTVGPSEAGGPSEAGGPSEAGGPSEAGGPSEAGGPSEAGGPSEAGGPSEAGGPSGTGWPSEAGGPSEAGGPSEAGGPSGTGWPSEAGWPSEAGWPSEAGWPSEAGWPSEAGWPSE

SEQ ID NO: 45 - DNA, 4223 bp, Babesia microti
ctcgtgcctttctcaactgataacagctaacaaaaagtctcttatcttaaaccatcctatacctcgtattataatatgaaaagggccttttctaaatctttccccaaagttctgctatttaattaaaaaaaaaaaagactcattcaataaacgggtggggcagaaagggtacctttccaagtgttcttccatgacgacccacaatgcaaagttcttcttacaaagaaaagagaaagatccactgagtgataagtaacccagctggggccgggcggtggtggcgcacacctttaatcccagcactcgggaggcagaggcaggcggatctctgtgagttcgagaccaggctggaccgacagcctccaaaacaatacagagaaaccctgtctcataaaaaaccaaaaaaaaagtaacccagctggatttggtaactgtctcagaaacagactatataaaacctcatcaccctacaacaagtaggaagctagcgctccccaccccatcccaacacacacacacacacacacacacacacacacacacacacacacacgcacacacgcacgcacgcacacacgcacgcacgcacacacgcacacacgcacgcacacacgcacacacgcacgcacgcacgcacgcacgcacgcacgcacgcccttctgtgtctgttctgttcaagaagggtaccacaaaaaagtaccttatggccacatcaatgacaattattactgtatataaaatgcccccatggatggcattgtattgtcgaaattaaaggcacccccgaaagaacagcacagaggggctaccaccaattaactcccaggaggaaataaagacagaagtgtgaaggagggagagagggagggaggaagggagggagaaaaggagggaaaggaacaaggagtaacagggacaaaagcagcagatggtgccaggcaggagtgtgcctaccacaccgggccttcccgttacttcatttactctcctttgcagcctgggaataaacaagtcacgcgtcacccggtgtctcaagctcagcatggcttgatctgagtgcccgtgtatgtgttcattctataactgatttaaggaacaactttctgctcattgcctctatcttctcaaacatttcgaagcagttattttttataagaaaatataaaacaggccgactaaattcgatctttctctccccagctgctagtttcttatctagctgctttaggcagtctccacagattgcagccaggcccctattctcaattccatctgacttctgacagcgctctccatttcttatttgcagcttagacatcttcactgagagcaggagtaattcattcaaatgacaatgaggtatctgaatatcacacaaacacttcaaattctgtttattggaaatagatctgctcctgccccatcataacaatcctttttatcttacttaacaggggcaagaaaatctttcacttcatttcctatcatctcaaatgagttcctgtacatgaatgacttaaggtaaccatatccaacaacttgaagccaaccagtccctggtcctactacagacgttagggaacatatgtgaaaacctggtgtacaacctaaatcataactagacagaagacagcactatttcctggtcacatagaaagcagaatagcatcctcacaccaatgaggaaaatgtcatgaaggcaggagagatcatgactgaggtgatacttttaccaaagacttgccagtgattaatttctcaattagttagcaaaaaatatggctctctagtgaatttgtgtccacaccattttccagatgttttgatgtcacttaaatcaatctaattatttaagttaaaaaatgttacagatcattgctttttttcttttttagaagacatcaaaacaataggatttctatgaaatattctcacttcacagctgtgtcagttaaagtgctttgggttatacataaagaaaacagactcaagaaagtaagaacaggaatttggagcttgcaacactgatgttctttgtaaaaagagagactttatccagggattagattctgtcacaaggcctggaactctctcttctcagccttatttccccaatatggattagaatcttacactgcaagcttcccacaagggtggacaggtcctcaccatttgtttcagcaggaaaaagagtctgtatgcatccgtgatatctaagtcacaattccagaagtgagctttcctggctcctattggtcggacttaggtcaggtgtcacatttccttttggattagtctgtgattaatgaatgggcccactttgctcacccattaagacaataggcttccattctcgaagctggaagcatgacatgtcccacagaaactgtaataagagagaacataggttgctgtgtggagaaacgaggcaaccggcaagtcataagatgacaaagtcttggaaagtctaagtcagtggttctcagccttccctaaaccctaaaccctaaaccctaaaccctaaaccctaaaccctaaacccctaaaccctaaaccctaaaccctaaaccctaaaccctaaccctaaaccctaaaccctaaaccctaaaccctaaaccctaaccctaaccctaaccctaaccctaacctagccttcattgacgtctatccccaatcttagaaaaatcttcaaatcgattctagaataactggaagcaattatcagaaattgtataactgcttattagcttattagcttattagttaggatgtatgcacattgatgacaactagatgcagcaccacaatcactaccacgtaccaatcatataccaataatgtactaataatgtaccaataactatggtttataaagatggtgtcatttaaatcaatattagttccttatattacactctttttaatgagcggtgctgtctttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactgttgggcctagtgaagctggtgggcctagtgaagctggtgggcctagtgaagctggtgggcctagtgaagctggttggcctagtgaagctggttggcctagtgaagctggttggcctagtgaagctggttggcctagtgaagctggttggcctagtgaagctggttggcctagtgaacgatttggatatcagcttctttggtattctagaagaatagttatatttaatgaaatttatttatctcatatatacgaacatagtgttatgatattggaacgagatagggtgaacgatggtcataaagactacattgaagaaaaaaccaaggagaagaataaattgaaaaaagaattggaaaaatgttttcctgaacaatattcccttatgaagaaagaagaattggctagaataattgataatgcatccactatctcttcaaaatataagttattggttgatgaaatatccaacaaagcctatggtacattggaaggtccagctgctgatgattttgaccatttccgtaatatatggaagtctattgtacctaaaaatatgtttctatattgtgacttattattaaaacatttaatccgtttaacccccagaaagagctgaccagacaaaggttaactcttgaatcccaggcatcagcctgggaatccatcatgggactgatcaagaccccctgaatgtgggtgtcagtgaggaggcctaggtaatctattgagcctcgggcagcagatcagtacccatcccaattatacacaattgcagtgttgtggtttcacagtgaataattgtaggtcacagtccattatattgatgtcacagtttttaattgtcatgtcacagtgcaagctagtgatgtcagagtgtataactgtgttcatagagaatgtattgatgtcacagtcaataatcgtgatgtcatagtgcagtatattgatgtcacaatgtataattgtgatgttaaagtgcaagatagtgaagtcacagtatataattgtgatgtcatattgcattataatgatgtcacactttataattttttacatacagcactatagtgatgtaacagccaataattgtgatg

SEQ ID NO: 46 - protein, 294 aa, Babesia microti
LWFIKMVSFKSILVPYITLFLMSGAVFAGDTDREAGGPSGTVGPSEAGGPSEAGGPSEAGGPSEAGGPSEAGGPSEAGGPSEAGGPSGTVGPSEAGGPSEAGGPSEAGGPSEAGWPSEAGWPSEAGWPSEAGWPSEAGWPSEAGWPSERFGYQLLWYSRRIVIFNEIYLSHIYEHSVMILERDRVNDGHKDYIEEKTKEKNKLKKELEKCFPEQYSLMKKEELARIIDNASTISSKYKLLVDEISNKAYGTLEGPAADDFDHFRNIWKSIVPKNNFLYCDLLLKHLIRLTPRKS

SEQ ID NO: 47 - protein, 30 aa, Artificial Sequence
  Synthetic peptide of repeat region of antigen BMNI-3 (SEQ ID NO3)
SEAGGPSEAGGPSGTGWTSGTGWPSEAGWS

SEQ ID NO: 48 - protein, 30 aa, Artificial Sequence
  Synthetic peptide of repeat region of antigen BMNI-3 (SEQ ID NO3)
EAGGPSGTVGPSGTGWPSEAGWGSEAGWSS

SEQ ID NO: 49 - protein, 367 aa, Babesia microti
MVSFKSILVPYITLFLMSGAVFASDTDPEAGGPSEAGGPSGTVGPSEAGGPSEAGGPSGTGWPSEAGGPSEAGGPSEAGGPSEAGGPSGTGSEAGGWPSGTGWPSEAGWSSERFGYQLLPYSRRIVIFNEVCLSYIYKHSVMILERDRVNDGHKDYIEEKTKEKNKLKKELEKCFPEQYSLMKKEELARIFDNASTISSKYKLLVDEISNKAYGTLEGPAADNFDHFRNIWKSIVLKDMFIYCDLLLQHLIYKFYYDNTVNDIKKNFDESKSKALVLRDKITKKDGDYNTHFEDMIKELNSAAEEFNKIVDIMISNIGDYDEYDSIASFKPFLSMITEITKITKVSNVIIPGIKALTLTVFLIFITK

SEQ ID NO: 50 - DNA, 1908 bp, Babesia microti
aaaagatttaatgaacatactgacatgaatggtattcattattattatattgatggtagtttacttgcgagtggcgaagttacatctaattttcgttatatttctaaagaatatgaatatgagcatacagaattagcaaaagagcattgcaagaaagaaaaatgtgtaaatgtggataacattgaggataataatttgaaaatatatgcgaaacagtttaaatctgtagttactactccagctgatgtagcgggtgtgtcagatggattttttatacgtggccaaaatcttggtgctgtgggcagtgtaaatgaacaacctaatactgttggtatgagtttagaacaattcatcaagaacgagctttattcttttagtaatgaaatttatcatacaatatctagtcaaatcagtaattctttcttaataatgatgtctgatgcaattgttaaacatgataactatattttaaaaaaagaaggtgaaggctgtgaacaaatctacaattatgaggaatttatagaaaagttgaggggtgctagaagtgaggggaataatatgtttcaggaagctctgataaggtttaggaatgctagtagtgaagaaatggttaatgctgcaagttatctatccgccgcccttttcagatataaggaatttgatgatgaattattcaaaaaggccaacgataattttggacgcgatgatggatatgattttgattatataaatacaaagaaagagttagttatacttgccagtgtgttggatggtttggatttaataatggaacgtttgatcgaaaatttcagtgatgtcaataatacagatgatattaagaaggcatttgacgaatgcaaatctaatgctattatattgaagaaaaagatacttgacaatgatgaagattataagattaattttagggaaatggtgaatgaagtaacatgtgcaaacacaaaatttgaagccctaaatgatttgataatttccgactgtgagaaaaaaggtattaagataaacagagatgtgatttcaagctacaaattgcttctttccacaatcacctatattgttggagctggagttgaagctgtaactgttagtgtgtctgctacatctaatggaactgaatctggtggagctggtagtggaactggaactagtgtgtctgctacatctactttaactggtaatggtggaactgaatctggtggaacagctggaactactacgtctagtggaactgaagctggtggaactagtggaactactacgtctagtggagctgctagtggtaaagctggaactggaacagctggaactactacgtctagtgaaggtgctggtagtgataaagctggaactggaactagtggaactactacgtctagtggaactggtgctggtggagctggtagtggtggacctagtggacatgcttctaatgcaaaaattcctggaataatgacactaactctatttgcattattaacatttattgtaaattgaatgaaacacatgatttatacattattatatattacaaaatttacacattatttatgtatgaacgaacgaacatcttgctcttaaataaagaaattgagatatatatggaaatagattaaagtaacatgagaaagatgaatataatattagaatatgaaatttaacagaaataaaatgaagtaaaagagtgtattttgtaataatttataataaattagtatacaatgattatattacaaatggctattaaatattttattaattaaatattgattagtaatgatattatgtatgtacatgttagggttgattgttatacattgtgaatatattatataattgtatattatattgattgatataatgtagaggatatttttttaaatagtatttaat

SEQ ID NO: 51 - DNA, 1460 bp, Babesia microti
aatccaacatctagcctagttagtatatataggttaatatcacattatagattatctttggatgattggttattatataacatgtcgctgaatgacgattattttgctagataatataactaccggtgattctgaggacctactttaaagagaataattaacatatctaccagaatcagttccaatttatgtattttaaagctaatcactactcgaaaactacggtgaaaatggaaaaacaagtggaagctgtatgtcgtggaaagtcactacattttatgtgggcaaatttaataattctaaatactatgtttttgatgttaaaaagcgaaaaacacactttaatgcacattttaacatcatctgtataatatatatatcagcgttgaaatcatatggcaaaggtaataaagcgttacattttgagcgaataaaggcacatatgcaaacgtatgaagccttgtatatttgtggaattatattatgctagtaatttgtgattaataatggcaatatttatatacaaatattcgagcgttctattatatgcatgcacataattaatcacaaactctcatatcatggggcggtttcgcccatcataaacattactgttagcactctggtagattagcatggtgaatctctcgatacctgggctactgttgctttccgcatattccttaaattctgcaagtgcgggggatgtatatgagatatcttctggtaatccacccgacatagagccaacatctacttctctagaaacaaatgtagttaccaactatattccagaacccaatgcggattcagaatctgtacatgttgaaatccaggaacatgataacatcaatccacaagacgcttgcgatagtgagccgctcgaacaaatggattctgataccagggtgttgcccgaaagtttggatgagggggtaccacaccaattctctagattagggcaccactcagacatggcatctgatataaatgatgaagaaccatcatttaaaatcggcgagaatgacataattcaaccaccctgggaagatacagctccataccattcaatagatgatgaagagcttgacaacttaatgagactaacggcgcaagaaacaagtgacgatcatgaagaagggaatggcaaactcaatacgaataaaagtgagaagactgaaagaaaatcgcatgatactcagacaccgcaagaaatatatgaagagcttgacaacttactgagactaacggcacaagaaatatatgaagagcgtaaagaagggcatggcaaacccaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaccgcaggaaacaagtgac

SEQ ID NO: 52 - protein, 503 aa, Babesia microti
KRFNEHTDMNGIHYYYIDGSLLASGEVTSNFRYISKEYEYEHTELAKEHCKKEKCVNVDNIEDNNLKIYAKQFKSVVTTPADVAGVSDGFFIRGQNLGAVGSVNEQPNTVGMSLEQFIKNELYSFSNEIYHTISSQISNSFLIMMSDAIVKHDNYILKKEGEGCEQIYNYEEFIEKLRGARSEGNNMFQEALIRFRNASSEEMVNAASYLSAALFRYKEFDDELFKKANDNFGRDDGYDFDYINTKKELVILASVLDGLDLIMERLIENFSDVNNTDDIKKAFDECKSNAIILKKKILDNDEDYKINFREMVNEVTCANTKFEALNDLIISDCEKKGIKINRDVISSYKLLLSTITYIVGAGVEAVTVSVSATSNGTESGGAGSGTGTSVSATSTLTGNGGTESGGTAGTTTSSGTEAGGTSGTTTSSGAASGKAGTGTAGTTTSSEGAGSDKAGTGTSGTTTSSGTGAGGAGSGGPSGHASNAKIPGIMTLTLFALLTFIVN

SEQ ID NO: 53 - protein, 275 aa, Babesia microti
MVNLSIPGLLLLSAYSLNSASAGDVYEISSGNPPDIEPTSTSLETNVVTNYIPEPNADSESVHVEIQEHDNINPQDACDSEPLEQMDSDTRVLPESLDEGVPHQFSRLGHHSDMASDINDEEPSFKIGENDIIQPRWEDTAPYHSIDDEELDNLMRLTAQETSDDHEEGNGKLNTNKSEKTERKSHDTQTPQEIYEELDNLLRLTAQEIYEERKEGHGKPNTNKSEKAERKSHDTQTTQEICEECEEGHDKINKNKSGNAGIKSYDTQTPQETSD

SEQ ID NO: 54 - DNA, 22 bp, Artificial Sequence
  PCR Primer
tttgcaggtgataccgatcgcg

SEQ ID NO: 55 - DNA, 24 bp, Artificial Sequence
  PCR Primer
tggtattctagaagaatagttata

SEQ ID NO: 56 - DNA, 306 bp, Babesia microti
ttgcaggtgataccgatcgcgaagctggtgggcctagtggaactgttgggcccagtgaagctggtgggcctagtgaagctggtgggcctagtggaactgttgggcccagtgaagctggtgggcctagtgaagctggtgggcctagtggaactggttggcctagtgaagctggtgggcctagtggaactgttgggcccagtgaagctggtgggcctagtgaagctggtgggcctagtggaactggttggcctagtggaactggttggcctagtgaagttggttggcccattgaaccatttggatatc

SEQ ID NO: 57 - DNA, 318 bp, Babesia microti
ttgcaggtgataccgatcgcgaagctggtgggcctagtggaactgttgggcccagtgaagctggtgggcctagtgaagctggtgggcctagtggaactgttgggcccagtgaagctggtgggcctagtgaagctggtgggcctagtggaactggttggcctagtgaagctggtgggcctagtggaactgttgggcccagtgaagctggtgggcctagtgaagctggtgggcctagtggaactggttggcctagtggaactggttggcctagtgaagttggttggcctaatgaaccatttggatatcaccttctttggt

SEQ ID NO: 58 - DNA, 358 bp, Babesia microti
ttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggttggcctagtgaagctggttggcctagtgaagctggtgggcctagtggaactggttggcctagtgaagctggttggcctagtgaagctggttggcctagtgaagctggttggcctagtgaagctggttggcc

SEQ ID NO: 59 - DNA, 409 bp, Babesia microti
tgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggttggcctagtgaagctggttggcctagtgaagctggtgggcctagtggaactggttggcctagtgaagctggttggcctagtgaagctggttggcctagtgaagctggttggcctagtgaagctggttggcctagtgaacgatttggatatcagcttctttggtattctagaagaatagttata

SEQ ID NO: 60 - DNA, 351 bp, Babesia microti
gtgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggttggcctagtgaagctggttggcctagtgaagctggtgggcctagtggaactggttggcctagtgaagctggttggcctagtgaagctggttggcctagtgaagctggttggcctagtgaagctggttggcctagtgaacgat

SEQ ID NO: 61 - DNA, 410 bp, Babesia microti
aggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggttggcctagtgaagctggttggcctagtgaagctggtgggcctagtggaactggttggcctagtgaagctggttggcctagtgaagctggttggcctagtgaagctggttggcctagtgaagctggttggcctagtgaacgatttggatatcagcttctttggtattcta

SEQ ID NO: 62 - DNA, 416 bp, Babesia microti
ttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggttggcctagtgaagctggttggcctagtgaagctggtgggcctagtggaactggttggcctagtgaagctggttggcctagtgaagctggttggcctagtgaagctggttggcctagtgaagctggttggcctagtgaacgatttggatatcag

SEQ ID NO: 63 - DNA, 356 bp, Babesia microti
ttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactggttggcctagtgaagctggttggcctagtgaagctggttggcctagtgaagctggttggcctagtgaagctggttggcctagtgaagctggttggcctagtgaacgatttggatatcagcttctttggtattctagaagaatagttata

SEQ ID NO: 64 - DNA, 285 bp, Babesia microti
ttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactggttggcctagtgaagctggttggcctagtgaagctggttggcctagtgaagctggttggcctagtgaagctggttggcctagtgaagctggttggc

SEQ ID NO: 65 - DNA, 342 bp, Babesia microti
ttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactggttggcctagtgaagctggttggcctagtgaagctggttggcctagtgaagctggttggcctagtgaagctggttggcctagtgaagctggttggcctagtgaacgatttggatatcagcttctttggtattcta

SEQ ID NO: 66 - DNA, 363 bp, Babesia microti
ttgcaggtgataccgatcgcgaagctggtgggcctagtggaactgttgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtgaagctggtgggcctagtggaactggttggcctagtgaagctggttggcctagtgaagctggttggcctagtgaagctggttggcctagtgaagctggttggcctagtgaagctggttggcctagtgaacgatttggatatcagcttctttggtattctagaa

SEQ ID NO: 67 - DNA, 363 bp, Babesia microti; identical to SEQ ID NO: 66

SEQ ID NO: 68 - protein, 101 aa, Babesia microti
AGDTDREAGGPSGTVGPSEAGGPSEAGGPSGTVGPSEAGGPSEAGGPSGTGWPSEAGGPSGTVGPSEAGGPSEAGGPSGTGWPSGTGWPSEVGWPIEPFGY

SEQ ID NO: 69 - protein, 105 aa, Babesia microti
AGDTDREAGGPSGTVGPSEAGGPSEAGGPSGTVGPSEAGGPSEAGGPSGTGWPSEAGGPSGTVGPSEAGGPSEAGGPSGTGWPSGTGWPSEVGWPNEPFGYHLLW

SEQ ID NO: 70 - protein, 118 aa, Babesia microti
AGDTDREAGGPSGTVGPSEAGGPSEAGGPSEAGGPSEAGGPSEAGGPSEAGGPSEAGGPSEAGGPSEAGGPSEAGWPSEAGWPSEAGGPSGTGWPSEAGWPSEAGWPSEAGWPSEAGW

SEQ ID NO: 71 - protein, 136 aa, Babesia microti
AGDTDREAGGPSGTVGPSEAGGPSEAGGPSEAGGPSEAGGPSEAGGPSEAGGPSEAGGPSEAGGPSEAGGPSEAGWPSEAGWPSEAGGPSGTGWPSEAGWPSEAGWPSEAGWPSEAGWPSERFGYQLLWYSRRIVI

SEQ ID NO: 72 - protein, 116 aa, Babesia microti
EAGGPSGTVGPSEAGGPSEAGGPSEAGGPSEAGGPSEAGGPSEAGGPSEAGGPSEAGGPSEAGGPSEAGWPSEAGWPSEAGGPSGTGWPSEAGWPSEAGWPSEAGWPSEAGWPSER

SEQ ID NO: 73 - protein, 136 aa, Babesia microti
GDTDREAGGPSGTVGPSEAGGPSEAGGPSEAGGPSEAGGPSEAGGPSEAGGPSEAGGPSEAGGPSEAGGPSEAGGPSEAGWPSEAGWPSEAGGPSGTGWPSEAGWPSEAGWPSEAGWPSEAGWPSERFGYQLLWYS

SEQ ID NO: 74 - protein, 138 aa, Babesia microti
AGDTDREAGGPSGTVGPSEAGGPSEAGGPSEAGGPSEAGGPSEAGGPSEAGGPSEAGGPSEAGGPSEAGGPSEAGGPSEAGGPSEAGWPSEAGWPSEAGGPSGTGWPSEAGWPSEAGWPSEAGWPSEAGWPSERFGYQ

SEQ ID NO: 75 - protein, 118 aa, Babesia microti
AGDTDREAGGPSGTVGPSEAGGPSEAGGPSEAGGPSEAGGPSEAGGPSEAGGPSEAGGPSEAGGPSGTGWPSEAGWPSEAGWPSEAGWPSEAGWPSEAGWPSERFGYQLLWYSRRIVI

SEQ ID NO: 76 - protein, 94 aa, Babesia microti
AGDTDREAGGPSGTVGPSEAGGPSEAGGPSEAGGPSEAGGPSEAGGPSEAGGPSEAGGPSGTGWPSEAGWPSEAGWPSEAGWPSEAGWPSEAGW

SEQ ID NO: 77 - protein, 113 aa, Babesia microti
AGDTDREAGGPSGTVGPSEAGGPSEAGGPSEAGGPSEAGGPSEAGGPSEAGGPSEAGGPSEAGGPSGTGWPSEAGWPSEAGWPSEAGWPSEAGWPSEAGWPSERFGYQLLWYS

SEQ ID NO: 78 - protein, 120 aa, Babesia microti
AGDTDREAGGPSGTVGPSEAGGPSEAGGPSEAGGPSEAGGPSEAGGPSEAGGPSEAGGPSEAGGPSEAGGPSGTGWPSEAGWPSEAGWPSEAGWPSEAGWPSEAGWPSERFGYQLLWYSR

SEQ ID NO: 79 - protein, 120 aa, Babesia microti; identical to SEQ ID NO: 78

SEQ ID NO: 80 - DNA, 29 bp, Artificial Sequence
  PCR Primer
cagagcagtactgatgatattaagaaggc

SEQ ID NO: 81 - DNA, 43 bp, Artificial Sequence
  PCR Primer
caatatgaattcagtgaatatttacaataaatgttaataatgc

SEQ ID NO: 82 - DNA, 32 bp, Artificial Sequence
  PCR Primer
cataacaatattccagaacccaatgcggattc

SEQ ID NO: 83 - DNA, 32 bp, Artificial Sequence
  PCR Primer
cgctagaattcattagaaagccttaaacatgc

SEQ ID NO: 84 - DNA, 2001 bp, Babesia
atgcagcatcaccaccatcaccacactgatgatattaagaaggcatttgacgaatgcaaatctaatgctattatattgaagaaaaagatacttgacaatgatgaagattataagattaattttagggaaatggtgaatgaagtaacatgtgcaaacacaaaatttgaagccctaaatgatttgataatttccgactgtgagaaaaaaggtattaagataaacagagatgtgatttcaagctacaaattgcttctttccacaatcacctatattgttggagctggagttgaagctgtaactgttagtgtgtctgctacatctaatggaactgaatctggtggagctggtagtggaactggaactagtgtgtctgctacatctactttaactggtaatggtggaactgaatctggtggaacagctggaactactacgtctagtggaactgaagctggtggaactagtggaactactacgtctagtggagctgctagtggtaaagctggaactggaacagctggaactactacgtctagtgaaggtgctggtagtgataaagctggaactggaactagtggaactactacgtctagtggaactggtgctggtggagctggtagtggtggacctagtggacatgcttctaatgcaaaaattcctggaataatgacactaactctatttgcattattaacatttattgtaaatattccagaacccaatgcggattcagaatctgtacatgttgaaatccaggaacatgataacatcaatccacaagacgcttgcgatagtgagccgctcgaacaaatggattctgataccagggtgttgcccgaaagtttggatgagggggtaccacaccaattctctagattagggcaccactcagacatggcatctgatataaatgatgaagaaccatcatttaaaatcggcgagaatgacataattcaaccaccctgggaagatacagctccataccattcaatagatgatgaagagcttgacaacttaatgagactaacggcgcaagaaacaagtgacgatcatgaagaagggaatggcaaactcaatacgaataaaagtgagaagactgaaagaaaatcgcatgatactcagacaccgcaagaaatatatgaagagcttgacaacttactgagactaacggcacaagaaatatatgaagagcgtaaagaagggcatggcaaacccaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagggcatgacaaaatcaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagagcatggcaatctcaataagaataaaagtgggaaggctggaataaaatcgcataatactcagacaccgctgaaaaaaaaagacttttgtaaagaagggtgtcatggttgcaataataagcccgaggataatgaaagagacccgtcgtcgcctgatgatgatggtggctgcgaatgcggcatgacgaatcactttgtctttgactacaagacaacactcttgttaaagagcctcaagactgaaacatccactcattattacattgccatggctgcaatttttactatttcattattcccatgcatgtttaaggctttctga

SEQ ID NO: 85 - protein, 666 aa, Babesia
MQHHHHHHTDDIKKAFDECKSNAIILKKKILDNDEDYKINFREMVNEVTCANTKFEALNDLIISDCEKKGIKINRDVISSYKLLLSTITYIVGAGVEAVTVSVSATSNGTESGGAGSGTGTSVSATSTLTGNGGTESGGTAGTTTSSGTEAGGTSGTTTSSGAASGKAGTGTAGTTTSSEGAGSDKAGTGTSGTTTSSGTGAGGAGSGGPSGHASNAKIPGIMTLTLFALLTFIVNIPEPNADSESVHVEIQEHDNINPQDACDSEPLEQMDSDTRVLPESLDEGVPHQFSRLGHHSDMASDINDEEPSFKIGENDIIQPPWEDTAPYHSIDDEELDNLMRLTAQETSDDHEEGNGKLNTNKSEKTERKSHDTQTPQEIYEELDNLLRLTAQEIYEERKEGHGKPNTNKSEKAERKSHDTQTTQEICEECEEGHDKINKNKSGNAGIKSYDTQTTQEICEECEEGHDKINKNKSGNAGIKSYDTQTPQETSDAHEEGHDKINTNKSEKAERKSHDTQTTQEICEECEEGHDKINKNKSGNAGIKSYDTQTPQETSDAHEEEHGNLNKNKSGKAGIKSHNTQTPLKKKDFCKEGCHGCNNKPEDNERDPSSPDDDGGCECGMTNHFVFDYKTTLLLKSLKTETSTHYYIAMAAIFTISLFPCMFKAF

SEQ ID NO: 86 - DNA, 3402 bp, Babesia
atgcagcatcaccaccatcaccacttgacttttggaaatatacgttttcataatataaatctcccaccattttcattgggcataattcactcgattacggtagaaaaggcgattaactctgaagattttgacggaatacaaacacttttacaagtgtctatcattgctagttacggtccatctggcgattacagtagttttgtgttcactccagttgtaacagcagacaccaacgttttttacaaattagagacggatttcaaacttgatgttgatgttattactaagacatcactagaattgcccacaagtgttcctggctttcactacaccgaaactatttaccaaggcacagaattgtcaaaatttagcaagcctcagtgcaaacttaacgatcctcctattacaacaggatcggggttgcaaataatacatgatggtttgaataattcgacaattataaccaacaaagaagttaatgtggatggaacagatttagttttttttgaattgctccctccatcggatggcattcccaccttgcgatcaaaattatttcccgtcctgaaatcaattccaatgatatctaccggggttaatgaattactgttggaagtactcgagaacccctctttccctagtgcaattagcaattacaccggactgacaggccgacttaacaaattacttacagttttagacggtattgttgatagcgccattagtgtcaagactacagaaactgtccctgacgacgcagaaacttctatttcttcattgaaatcattgataaaggcaatacgagataatattactaccactcgaaacgaagttaccaaagatgatgtttatgcattgaagaaggccctcacttgtctaacgacacacctaatatatcattcaaaagtagatggtatatcattcgacatgctgggaacacaaaaaaataaatctagcccactaggcaagatcggaacgtctatggacgatattatagccatgttttcgaatcccaatatgtatcttgtgaaggtggcgtacttgcaagccattgaacacatttttctcatatcaaccaaatacaatgatatatttgattacaccattgattttagtaagcgtgaagctactgattctggatcatttaccgatatattgctcggaaacaaggtgaaggaatctttgtcatttattgagggtttgatttctgacataaaatctcactcattgaaagctggggttacaggaggtatatcaagttcatcattatttgatgaaatcttcgacgagttaaatttggatcaagcaacaattagaacccttgttgcaccattagattggccacttatctcagacaaaagcctccacccttcactgaagatggttgtggtcctgccaggatttttcatagttcctggatccactgatgatattaagaaggcatttgacgaatgcaaatctaatgctattatattgaagaaaaagatacttgacaatgatgaagattataagattaattttagggaaatggtgaatgaagtaacatgtgcaaacacaaaatttgaagccctaaatgatttgataatttccgactgtgagaaaaaaggtattaagataaacagagatgtgatttcaagctacaaattgcttctttccacaatcacctatattgttggagctggagttgaagctgtaactgttagtgtgtctgctacatctaatggaactgaatctggtggagctggtagtggaactggaactagtgtgtctgctacatctactttaactggtaatggtggaactgaatctggtggaacagctggaactactacgtctagtggaactgaagctggtggaactagtggaactactacgtctagtggagctgctagtggtaaagctggaactggaacagctggaactactacgtctagtgaaggtgctggtagtgataaagctggaactggaactagtggaactactacgtctagtggaactggtgctggtggagctggtagtggtggacctagtggacatgcttctaatgcaaaaattcctggaataatgacactaactctatttgcattattaacatttattgtaaatattccagaacccaatgcggattcagaatctgtacatgttgaaatccaggaacatgataacatcaatccacaagacgcttgcgatagtgagccgctcgaacaaatggattctgataccagggtgttgcccgaaagtttggatgagggggtaccacaccaattctctagattagggcaccactcagacatggcatctgatataaatgatgaagaaccatcatttaaaatcggcgagaatgacataattcaaccaccctgggaagatacagctccataccattcaatagatgatgaagagcttgacaacttaatgagactaacggcgcaagaaacaagtgacgatcatgaagaagggaatggcaaactcaatacgaataaaagtgagaagactgaaagaaaatcgcatgatactcagacaccgcaagaaatatatgaagagcttgacaacttactgagactaacggcacaagaaatatatgaagagcgtaaagaagggcatggcaaacccaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagggcatgacaaaatcaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagagcatggcaatctcaataagaataaaagtgggaaggctggaataaaatcgcataatactcagacaccgctgaaaaaaaaagacttttgtaaagaagggtgtcatggttgcaataataagcccgaggataatgaaagagacccgtcgtcgcctgatgatgatggtggctgcgaatgcggcatgacgaatcactttgtctttgactacaagacaacactcttgttaaagagcctcaagactgaaacatccactcattattacattgccatggctgcaatttttactatttcattattcccatgcatgtttaaggctttctaatga

SEQ ID NO: 87 - protein, 1132 aa, Babesia
MQHHHHHHLTFGNIRFHNINLPPFSLGIIHSITVEKAINSEDFDGIQTLLQVSIIASYGPSGDYSSFVFTPVVTADTNVFYKLETDFKLDVDVITKTSLELPTSVPGFHYTETIYQGTELSKFSKPQCKLNDPPITTGSGLQIIHDGLNNSTIITNKEVNVDGTDLVFFELLPPSDGIPTLRSKLFPVLKSIPMISTGVNELLLEVLENPSFPSAISNYTGLTGRLNKLLTVLDGIVDSAISVKTTETVPDDAETSISSLKSLIKAIRDNITTTRNEVTKDDVYALKKALTCLTTHLIYHSKVDGISFDMLGTQKNKSSPLGKIGTSMDDIIAMFSNPNMYLVKVAYLQAIEHIFLISTKYNDIFDYTIDFSKREATDSGSFTDILLGNKVKESLSFIEGLISDIKSHSLKAGVTGGISSSSLFDEIFDELNLDQATIRTLVAPLDWPLISDKSLHPSLKMVVVLPGFFIVPGSTDDIKKAFDECKSNAIILKKKILDNDEDYKINFREMVNEVTCANTKFEALNDLIISDCEKKGIKINRDVISSYKLLLSTITYIVGAGVEAVTVSVSATSNGTESGGAGSGTGTSVSATSTLTGNGGTESGGTAGTTTSSGTEAGGTSGTTTSSGAASGKAGTGTAGTTTSSEGAGSDKAGTGTSGTTTSSGTGAGGAGSGGPSGHASNAKIPGIMTLTLFALLTFIVNIPEPNADSESVHVEIQEHDNINPQDACDSEPLEQMDSDTRVLPESLDEGVPHQFSRLGHHSDMASDINDEEPSFKIGENDIIQPPWEDTAPYHSIDDEELDNLMRLTAQETSDDHEEGNGKLNTNKSEKTERKSHDTQTPQEIYEELDNLLRLTAQEIYEERKEGHGKPNTNKSEKAERKSHDTQTTQEICEECEEGHDKINKNKSGNAGIKSYDTQTTQEICEECEEGHDKINKNKSGNAGIKSYDTQTPQETSDAHEEGHDKINTNKSEKAERKSHDTQTTQEICEECEEGHDKINKNKSGNAGIKSYDTQTPQETSDAHEEEHGNLNKNKSGKAGIKSHNTQTPLKKKDFCKEGCHGCNNKPEDNERDPSSPDDDGGCECGMTNHFVFDYKTTLLLKSLKTETSTHYYIAMAAIFTISLFPCMFKAF

SEQ ID NO: 88 - DNA, 29 bp, Artificial Sequence
  PCR Primer
ccgtcgcagctgacttttggaaatatacg

SEQ ID NO: 89 - DNA, 36 bp, Artificial Sequence
  PCR Primer
ctagaattcataggatccaggaactatgaaaaatcc

SEQ ID NO: 90 - DNA, 27 bp, Artificial Sequence
  PCR Primer
cgtggatccactgatgatattaagaag

SEQ ID NO: 91 - DNA, 1148 bp, Babesia microti
tgactggatatcgccaattgtattgtaacaggtggttgctatggaaatattaccaagctgcagcgctttatcgccgagtctcttccacctttcaatatttatgggttcaatttgcattttagacgcggattttctgggcgagtttacctcagatgaattatttggtgatttcaaggcacctttataaaatgtctcataagaatcaccattcaattgttccagacactaaatatttgtcaacttactaactcaacatcaccggtgctcaaagccaattcaaatttccttgatgcatttaccgttatggcaactgctagctgtatattacccatagcttcaactattacgcttatcctttcccgtaaatgggttggtatttcagcaaccatatcaagtactaatccccaattattctccaacattgcagtctaaatgaattgattggattacattcaacttcaaataagtttcagttaggttgtatgacagaacatttccatctcgatcaataagatatatcttgcttgtttcaatggaatagcccgagaatgtacagkttacgatctagataagcgtgcggatcaggcacgccagttgtgtacaaataagagtctcagaccaactgktgkgtaaataaatgtatcacaaacccatactccactcacaatacsktctgtaatctaggttaaacaacaatttaacctcagtatccagctcaaatgttgatggtactccctcgtcatcctgaggcgtattggcagcgactgataccaaaaaggaattatgatcatattcttctagttacgttttttctactgatggaatctttaccaaagttaccccagctacagggttttcaattggttgtgtaatatttggcaatcaattaattccacagtccatggatgttatcactaggaccgtttcatacaccactaaatatcctttgattgttgttaggattcaagataagacttcgagttctacttcaaccgtttactatgagcaatctggtttacaatctagcaaatttgttttgagggatgacccagaatttattattcctcaaaatcgaagtagtacttatacagtcaatgacataacatataaatcatttgatatttctagtgccgatgatacgaatttta

SEQ ID NO: 92 - DNA, 605 bp, Babesia microti
  misc_feature (1)...(605): n=A,T,C or G
tggccttgcctcaacttaatgtgccagggaggcattagcatttctgaggagtattcactcctcagtgtgtggtgggttgaggggaggtagggagaaagggagaaaggcagggaggggaaactctaggtgttatgaaaagtgtatataacattaaaattgagggtgagaagtaatgaggataaatgtaattacccataagaacttcggtccagcaactgaaaagtagtggaaacaactaaatgaacaaactctgagaaaaggagaccatgtttaataggaattcattcctacagaactatgaaacactggtacttggtacataagacaaactacagaaagtaggatacgaatgtcagagccttctttttattttttttctgagagatttgatcttgctcagagatgccaattgagttctatactccaataattgagcacttgtaccttgacctttaatatcctccggaaaaattatagatatgagggagtataggtatgagaaaattgtctcatttgtatcctgacctccmcttgtatcctgatctccacttgttgntgacccttcacttgtttgntgaccttcccttgtttggtgaccttccttg

SEQ ID NO: 93 - DNA, 631 bp, Babesia microti
gttcctactttgtcatcattggtcaagttgttcagtgaagttatgctgagagtgaaggatgcgtcttccacagaggctaccatacgcatgttcctccgtttcaacgcatttataaaatttttgaatgaggagaaatccagaggtgacaaaagtgcgttgaatgatgagggattgatgaggtttatatcgatgaccagtggatttatcgatgaccttgaattagttttagatgagttatccaagcacagtttgcttataaataacgaaggtgccaagagcatgctatcctctctcatactaagcttccgttatattaatcacataagaaatttgatcaatggtatttaccttggattgaataacccatcatcgtccattggtgagacagcacaagaaacaactgaaccctccactcccactcccactcccagcacacagacaatcctgaaaccgaagggatccgagataaggggctatataataaaagttgatcaaacagctaatctcatcacattcatagatgcattgatcaaggagttgaacgttcatattaaacagacaacaacttcgtctggtkgttggcactaaagaaactaatggcactaccagtggttctyctgaaagcaatccc

SEQ ID NO: 94 - DNA, 632 bp, Babesia microti
  misc_feature (1)...(632): n=A,T,C or G
ataaataagtaaatacttactgaaaacacttcaaaaacatgcaaaaacacagcataggacttaacaattacaaagtgaaactgtacaattccatccttctaatgccatttacaagttgagaatttaggaaatataaatcataagcagatagatcaaaaacagaatatctggaataatgaaacataaaatggaaatctaaactagaagtaagttttataaagccacaggcaggtactgaacctgagttcctggttaccgactgttagtcttcccttaatggggtagacttggctggccccaaagccactggtatcatcattctgtctttgcatgtcctgtgcaagggctcaaggtgtgctgctgtgtccagtttgctacaagagtactgaggctgagcccatatccccatggttatatggtgaacaatttccacatggagcattctccccagttcatcttccagaattcaatattgatgtatcagttcttaattcattgatgtaagtcaatctcccttaaattaaaaattaatagaaagcaatttctctaacgggcaactttctgcttgcgtgtaatatgtatgtgaaatctagattctgcngaggagaccaaaccagtntatttttgtgcct

SEQ ID NO: 95 - DNA, 426 bp, Babesia microti
  misc_feature (1)...(426): n=A,T,C or G
attttgtactgttcaaatgtgtaatatatttgtgaaagaagaaaataatttaagtcaagaggatgatgaaagggcagaagtaatacttgagataagcacttcacatcttacaattaaaactcttctgtgtctacctgcaaattcatgacagatgaaattaacttgntttctattcggtttctcctcttatttctgccagtattataatttcaggaaggaacatgcatcataaattacatgtaactttcatgttgcagtgatgctggtttctatttttgatctcatttgacagcagtaaagtcatacnaaaaataataaatacctctcatggagcttgccatttcctctgcatcttttttggggaagaantggcctgaaaagtaaagcgttaagactcacaaagtcaaaaactttcagatagaaccc

SEQ ID NO: 96 - DNA, 472 bp, Babesia microti
  misc_feature (1)...(472): n=A,T,C or G
aggtnacacatagaggagtgtggtcaattaaacactcaagcaccctatgtcttggtttgctctctattgctgtgataaacaccagagctaagcccaacttgaagttgtcacatggtctccacacaaatacacacacacacacaccacacacacacctattgtatgcacatgcacccccccccccttncaaaaaaaaaggancctctactctttaccagcaataaaaaatgaactaggtgaaaagaaaaccaaccttgcttcatcatttagtcatagaaaatgatactggggttggcatttactatcattaacctaaaataaatgtgtccctacctaagggtataaactgttatctggccttgtacagattttggatcttgaattcttttagngggttgcccaatagcattttaaggncccagaataaatagaccggatgaaatgggatgggctagagtagaatggaggctan

SEQ ID NO: 97 - DNA, 867 bp, Babesia microti
  misc_feature (1)...(867): n=A,T,C or G
ttaatattatgttcaccgaaacatcctgtagtatacaactcaaccaattcaccattaaatgataatttgatcagtgtatattgtgatgttatatttattggtattgttatctcaccaaccttaacttcgctgatgtaaattttggaatctggattattggtgtacaacatgctcccatcacttaatgatatttttaaaaattcgttatcatcggcactagaaatatcaaatgatttatatgttatgtcattgactgtataagtactacttcgattttgaggaataataaattctgggtcatccctcaaaacaaatttgctagattgnaaaccagattgctcatagtaaacgggtgaagtagaactcgaagtcttatcttgaatcctaacaacmatcaaaggatatttagtggtgtatgaaacggtcctagtgataacatycatggactgtggaattaattgattgccaaatattacacaaccaattgaaaaccctgtagctggggtaactttggtaaagattccatcagtagaaaaaacgtaactagaagaaagaccctctggaacttgatcaacaaatcctatttcgtttatgttaagattcacaatatttgtgacagcaacatcttgtgtggtctccagagacggagaaattgttgatgtggcagctgttgttgatgtggtagctgttgttgatgtggcagttgttgttgatgtggcagttgttgttgatgtggtagctgttgttgatgtagcagatgttgttgatgtagcagtacatactgacagtacatgtgcatgtgtgtgtaaataggattcttgtaaagccaagtatatcctcactgctgatttgtctgatattacctcc

SEQ ID NO: 98 - DNA, 815 bp, Babesia microti
tagtcattagattatcatgacaccaataagctttttatcttgaagttgttttatatattaatacaaccatagcatcataaaagctacatttgttttttttatcttaacccatggtcatctagtctttttcctttattattcatcattgattgtccttaaatgctcaaagcatctgcccctttaaactacttctttctaaattagcatatactctatatggtcatacctattctgtgtaatcatcaggttccctgtgcaggggaaaggaggaacgctcaagcactgaggaatcatcccgctgtgtgataacgttgatggaagacaagtgatacagttagttgttcaaacaaataagcatattttaaggggaagaatagtgtcgtactaactaaaatctaatttgaccataatacgcacattagtttgtttgtgctcaatttttttaatgaatcaggcccccgatttatatttgtgaaagtccatgtgggagcgtaaggatgggatagtttatttacagtagcttctctggggaaaggaaagcaaagccccaactgtatagagttcattggagctgtcacctacgcccctgccttcctgtccctttagagtgcctcagtttgctgtgtggcaagagtctctccctgctcctgctctcctagccccctctgcctgcctcccccagttgatgcgagagtccactgttggagaagttaactctaatcttacacctggggagagctactggaaattaattttccatgtaactggctttgagttctagcaggctttagattttagaagtttttgtgtgtgtg

SEQ ID NO: 99 - DNA, 1225 bp, Babesia microti
  misc_feature (1)...(1225): n=A,T,C or G
attgtgtaaagggttaccatggccatggcaatttttgtaaaagaaagcatttaaatgggggcttgtttacagtttaagagggttgactcatgaccatcattgtgggaagcatggtagcaggttggcatggtgctggatcagtaattgagagctttacactctgatcctaaggcatcagacaaagaaaagcctggtcctggtgtgggcttgaagcctcaaaatccccctctaatgacacacttccctcaagtacatacttattcctaaatccttctcaaacagtttcaaaacttgtgcctgagtgttcaaatatatgaacctacaggggccattcacattcaaattatcacaggcagataagttactagtcatggaagttcaaatatatactttgttatgaaaatataaatatgctttagaatctggggaacccagaagggtggagatggggtcaagattctctgagatggggtcaagattctctgtgtctccctgggcctggctggaatgtccctcctgtcttccaagtcctctgttccaggtgaccatgtccccatcccagtcccctcgatggtcctcatgccctcctctcagttcctggctgctcccccacccccgccacatccccatcaagggactggccggctctcatactgctacccatgcagggtgctcatgcccttgcgccccggcacctttagtgtttcngtcccttcccggccccactcagcgccaccccatgtcgcagggccgccgtccgcgccacgggaccttgcaagtacaagcttgagccgcttcccccctggcgytgcgactgcggtggctgccgccttgcgggamtccggcggtcgttccgacgtcacctactcgstgcttgtgcctgctctgcgggccgcgacggtccggcgggcgcatgccaaccctgcgggccacgcgtggccttcgtcccgcgtcaggcagggttgcgagaacgcgccgccacgsttgctgcacctgcggccgggcgcgcgctataccgtgcgcgtggccgcgctcaacggtgtctcaggcccagcggccgccgcggaagccacctacgcgcaggtcaccgtgtccaccggacccggaggtgaggccacgcgccccagcggagtccgtccccctccccaaccgcagttccctctatgcattccaagtcattcaggaacccacgtgactacaccccatgccccaggtgcggcacgag

SEQ ID NO: 100 - DNA, 537 bp, Babesia microti
  misc_feature (1)...(537): n=A,T,C or G
aaagaaagagaagaagggagagaagagcaaggggaatgaatgagagaggagagaagggaatagaagagaggggagggcagaggaggggaagcagaggggaggggaaaggaaggagaaagagaacagagacagagggaaggtcaggtacatcactgtccaagagatcacatattatccaagcmacggacagagctttaggaagtgtacagagaggcacctttcacccagtgtcctataatgaccatttctgcaaattctctagaacttagttccattctgcacaacccctccatacctgtcatcatgtgcttcacttactagcctcaagtaagctgttaagtgttccagtgttatatgccattctagtaccttcatccagtgactgatagaagcagagctaaacncccncagttaaacaataaactgaatccctagaacccmgtgaccgagagtgktctcataattcttaaaaagatgctattaaattttatcctgtatcatactacattatctttttttcttccttcccctcccccc

SEQ ID NO: 101 - DNA, 543 bp, Babesia microti
  misc_feature (1)...(543): n=A,T,C or G
acataacactagggacttggcattgcatatctgtaaatataattgaaaccaaaataaaatattggtgagttccataggttgggttgttcacagtgacatttaaaagtgaaattcttgagagctggtttggaggttctattaggggagtgcggtacttgtataccttggactgaagaccagtcctcctctattccgggaaggycgycctcttcgaccaagcatgcacttcaggatggacacacatggagtgttgagggaggaaagagatccccctaagccagatagatcaactaaatgaaccttggaaataaatggggtgacagatgtarcavcgagaatgccctcacatactgaaaatgaaataattamccmccwttagtttttccatytgatacctaggcmctytctaatttaattccamcattctkgaaaagtgksttttgaaagattggtgggcaacccccctaattantcccctnccaatggggtaaggccaaaaaaccaggggggaaattccaaaaattattgntttgtaaaggggaa

SEQ ID NO: 102 - DNA, 811 bp, Babesia microti
  misc_feature (1)...(811): n=A,T,C or G
tggataaggatgaagtcagttagaccaatactaattcattttattacattctttttaacaagtggaaatgtctttgcaggaaatggtgatgttaatcaatattcaagtgattttggacgagcattaaacgatcttatgatcgcttttaacgaggctaaaaaaatgtatgcaaaattttctgaacagatcacggacactatgattcatacctgcaaaaatagtattgatatactagaagcagatgagaagaatggtggtcataaaaattaccttgaaaagaaagaaattgagctcaaaagtaaaattgtggaatttaacgccattttttcaaacattgatttaaataatangtacggktaaaaatgaaataattaaactgcttaatgatatatccactatctctaccgatattaagtcaattgttgatgaaatatactataaggctcttggtacaattgaaggtgaaaatgctgaaaattttgagtatgaaattaagaaaaagaaagctgaactacttagaaacctgctgaatgataatattaaaccaattatggggatatttaactgagatatcaatatgccatccaattatatcaaataaagcgaatttatgatatcaagaaagcatttgaaaagcacgaattagaagctaatgttttgatatcccagatattagaaaatatcagaattttggcactaattttaatgacattttaaatgaagtgaatggngcaattgaagaatttaataaaactattggacgtcatgaataacaccattggggacccttggtattggtattgacagcggg

SEQ ID NO: 103 - DNA, 2966 bp, Babesia microti
ctcgtgccgaatgtcatttatgatctaataatattgtattatctctaatattatggtataatagatactgtgaaaataaattcaactggagataaggaaaccatttgtatagatattttatacaaattattatgaaataatctaaataaatgacaaaaaatcgattatacaaatcacattaatgacaaacaaacttgtatacatatattgattaacattacaaaactaaattataatatttagattgataattgttataatacttaacaatattctactttttaatataattttttattcaataatatactctttcatattttgtactattttatataatcatatatattatataattatatatatttgataattgaatatatcaataatgatgatatacatgaatatgcatatataccccatataatgttattatatttagtgcttacattattaattataaatatatttaaataattaaataataatgaaaattaacatagacaatataatattaatcaatttgataatattattgaatcgtaatgtagtatattgtgtggataaaaatgatgtttcattatggaaatcaaaacctataacaactgtcagtaccactaatgatactattacaaataaatacactagtactgtaattaatgccaattttgctagctaccgtgaatttgaggatagggaacctttaacaataggatttgaatacatgatcgataaatcacaacaagataaattatcacatccaaataaaattgataaaatcaaaatttctgattatataattgaatttgatgacaatgctaaattaccaactggtagtgttaatgatatatccatcattacttgcaagcataataatccagtattaattagattctcatgtttaatagaaggatctatctgctattatttctacttattgaataatgatacaaataaatggaataatcacaaattaaaatatgataaaacatacaatgaacatactgacaataatggtattaattattataaaatcgattatagtgaatctacagaacctactaccgaatctactacctgtttttgttttcgcaaaaaaaatcataaatctgagcgtaaagaattagaaaattataaatatgagggtacagaattagcaagaatacattgtaataaagggaaatgtgtaaaattgggtgacattaagataaaggataagaatttggaaatttatgtgaaacagttaatgtctgtaaatactccagtaaattttgacaaccctacatcgattaatctaccactgtcagtactaccaatgatactattacaaataaatacactggactataattaatgccaatattgttgagtactgtgattgaggatgaccttacaataggttagatccttagataaatcacaacaaaataaattatcacatccaaataaaattgataaaatcaaawtttytgattatataattgaatttgcacgagatgttaaattaacaacaattggtactgtcaatattatatatatctatacttgcaagcataataatccagtattagttgaatttatagtttctatagaagratcttactacaattacttctactcaatgaataatgatacaaataaatggaataatcataaaataaaatatgatacaagatttaatgaacatactgacatgaatggtattaattattatgaatatgtacttggtaaatgcagttcttatacttgtaaaaatgaatatgagcataaagaattagcaagaatacattgtaatgaagaaaaatgtgtaaatgtaaaggtagataacattgggaataaaaatttggaaatttatctaaaataatttaacgaagtgtaatatgtaaaatagtttaatgaagtataatattatttaaaataattcaaaatttcagaaattaatataattaattattataaatacaaaataattaattacaaaataacgtattattagccatttcagattgtaaatacatatttttacatatatttttattaaaactttcaaattaatgttttcatttttataagcattattataattatatactataattatcagtcatcaaataatatccaaagttatcctctacattatatcaatcatacagtatacaattatataaaatattaacaacatataacaaccaacattaatatatacataatatctttattaatcaatatttaatcaatacaataattaatagttaactaactatacacatagtgtatactaaattattataaattatatgttataattacaaaaacgtcatttacttattttatttcagttatgtttcatagtctaatttagatttggtgaaacgcatctggctgatgtgctggtgagcaagcagttccacgaagcaaacaatatgactgatgcgctggcggcgctttctgcggcggttgccgcacagctgccttgccgtgacgcgctgatgcaggagtacgacgacaagtggcatcagaacggtctggtgatggataaatggtttatcctgcaagccaccagcccggcggcgaatgtgctggagacggtgcgcggcctgttgcagcatcgctcatttaccatgagcaacccgaaccgtattcgttcgttgattggcgcgtttgcgggcagcaatccggcagcgttccatgccgaagatggcagcggttacctgttcctggtggaaatgcttaccgacctcaacagccgtaacccgcaggtggcttcacgtctgattgaaccgctgattcgcctgaaacgttacgatgccaaacgtcaggagaaaatgcgcgcggcgctggaacagttgaaagggctggaaaatctctctggcgatctgtacgagaagataactaaagcactggcttgataaataaccgaatggcggcaatagcgccgccattcggggaatttacccctgttttct

SEQ ID NO: 104 - DNA, 1137 bp, Babesia microti
gtttttttcccctgaggttttgattgttaatttaatgtcaaattaattggattaagaaatgccagcagagcatggtggtgaacacctctaattcccaggcaggtgaatctttgagttcaaggccaacctcatgtacaaacctagttcccagtatasccatgmytaamcagggaaaccgkgtctkgggaaaaamcaaaawtaaamcagaagagaaagggggaaatgcctggggattagtgaggttaatgccagtggtggtatttattaccagagacaataagaccgtgagagctctgggaattttgtttgtttgttttttgcttttccaagacagggtttcttggtagctttggagcctgtcctggaactcaggctatagatcaggctggcctcgaactcacagacatccacctgcctctgcctcccaaatgctgggattaaaggtgtgtgctaccaccacccgggctagaaagaacttgttagttgggatgtaaattctgggtcatccctcaaaacaaatttgctagattgtaaaccagattgctcatagtaaacggttgaagtagaactcgaagtcttatcttgaatcctaacaacaatcaaaggatatttagtggtgtatgaaacggtcctagtgataacatccatggactgtggaattaattgattgccaaatattacacaaccaattgaaaaccctgtagctggggtaactttggtaaagattccatcagtagaaaaaaccgtaactagaagaaagaccctctggaacttgatcaacaaatcctatttcgtttatgttaagattcacaatatttgtgacagcaacatcttgtgtggtctccagagacggagaaattgttgatgtggcagctgttgttgatgtggtagctgttgttgatgtggcagttgttgttgatgtggcagttgttgttgatgtggtagctgttgttgatgtagcagatgttgttgatgtagcagatgttgttgatgtagcagctgttgttgatgtagcagctgttgttgattgagcggcggttgctgctgaagtaggtattgaatttgctatactcacacttgtggcatcggtacctgcgcctcctctagtgtttgttgccaaagtcagagtgagcctgt

SEQ ID NO: 105 - DNA, 1010 bp, Babesia microti
taggaatatggatttgagctttgcctatggtatcatcccataggcatgagtcagggtcaaaatcgccagaatattccaggcaggttttagtaaccctatccatcaatggcgtgttaggggaaaccgaaggtatattatttgagttttcatccttagatatacagttttctaaggcataaggggttttcccgccagtgcttgtagtattggttattgacagtagttttttagttccactttcattagtgatagctgcggaggcttttgcgatagagctggctagtatagatgaagattttgagtctttgtttagggggaagtgaatggtgcaattgaagaatttaataaaactattgacgtcatgaataacaccattggggaccttggtattgttattgacagcggtattatttcaagcataaaatcatatatttccacaatcgccaagatttctaaagcaataatccctggacaaatggcattagtttttactgcattaatattaattctaaattaaatgaaattcagatgtatatattattatatagtacaaaatttacacatttattatatacatgaacgaacatcttgctcttaaataaagaaattgagatataaatggaaataaattaaaagtaacatgagaaagatgaatataatattaaaatattaaatttaactgaaataaaatgaaataaaagaatgtattttataataatttataataaattagtatacaatgattctacattataacaagcgagaataaataattattgattagtcataatattatgtatatgttaaggtttattgttatgtgttgctaatatgttatataattgtataccatagtgattgatataatgtagaggataactttggatattatttgatgactgataattatagtatataattataataatgkttataaaaatgacattaatttgaaagtttaaattaaaatatatgtaaaaatatgtatttaaatctgaaatggctaataa

SEQ ID NO: 106 - DNA, 1162 bp, Babesia microti
atgtgaatgcattgatcaaggagttgaacgctcatattaaacagagagcaacatctacaacaacaattattattgaaactaatgctaaagatgtggatgagttagtgaaaaaatttgcaacaattgcatcttttgatgataagttcaagaacgtattctttgataattctgttattgatgaaattgtcaaaacgttggaaaagatgaaggttgagtcagatactgtattacctagttgcaatggaatccagaccactgaaaactctagtactgacccatatacagtattatcaaaacttataaagaaaattaacgactccataatcagacctatgacttctcggctgatcaacaaatcctttccggagttgtgcaagttgtttataaaaatgcccgatgtcgactccacaaatttatggctttggatgtggacataagccamcactcttgtaamcagragagtcagatattctgatggcagatttaccattgtaagcactgggtccaattttagatacacattggcmccaactgccgcttggtcatgatttgtctctcttctcccaattgccaatctcaatgattacggcacatcgcctcaggagcaggcacttacatcttgcgtcagtcatggtaacgaattcagcatagtaagcactgcaggcaagacaacttacactacacaatctaagttgttgtcacttttcaagttatctgcggagacgttaagggattttaatgaagctagatttgcacttggtaacatgactgatagtgctaataaatctaaagctttggaggtctacaaatcgacactaacttactatgaaatcaatatcagtcgaattggaaaagatttttggcatattaaaatcaactccgaatattacttttgaatcagttgtttctaaatacaaattgactggtgttaatacagttgatactgccaatgctgatgtgatcaacgagacaatgtttgacgatttgtccaaggcaatttcctcatacctatactccctcatatctataatttttccggaggatattaaaggtcaaggtacaagtgaaggtcaacaaacaagtggaggtcaggatacaaatgagacaattttctcatacctatactccctcatatctataatttttccggaggatattaaag

SEQ ID NO: 107 - DNA, 984 bp, Babesia microti
tgggtgagctagctgttgtccagccttggtgtgattggacagtgtagagctcatctgaagtcttggcttgatagtgaggctggaccatctcagctagcagctttgaagctgttctggatgcagaattttgagggaactgcaacagaggctttctgagaggctggatcaattgggctactcatctgtattggtttctggtcctttttttctgaaagcacaaacttttaaaggtaccatatgtatctgcattagcacaatggaatgtgcagtgtgcacaggtcaactaaaggttttttcttctgtgtatgagcaggtaaaaggcacctgtcaactttataagtccaaaccttcgaaaatgatggcactatgacatcaaaattttattccagggagtccctagacccaacaacctacatcggacatgcacctacagacatatttacgtcgccatggatcacgacccacatgcataacaagcgtcttgttgactttgaagttccatttgaagcaatttttgatgataaactcataagttattataccggtacggatgtcaacggcaagaataaggttcctgcagagcttaccaaggcaatatgcggcaaagaagacgtgtgtgagcttaacattaccggtttattgttgaaagatattagtgctaagaaattggaggagtgtaggaagaagaatgcatctagtggtactccatctggtggtacaccttctaatgttccagaggagtgtgtgattaaaagcaacttacagacggttatgaagaaggatgttactacaactttgaaatcggatgatgtcagcaattacagtgttgtatccattcacttttacattgataacgtgttcagacataatactgcttttggcagaattaagattggcaaccttgatctaccagcattttccattgggtttatccactcgatcttcgtcgagagggttctcatgggtgacaagagccttgccagt

SEQ ID NO: 108 - DNA, 537 bp, Babesia microti
ttatggagggctatttagatctcgatttgaattccaagattggtaactttatttcagccatcgaactcactaacctgaccaacacggtaaaatcagcgagcgtccaccctccccaactaaaagtgttggctctgaagtttggcaacaagatcgttgatgtcgaggagacaggcaggacatttgttacatttgatgagaagttgaattcaatagaaataattaccttcgaaaatgatggcactatgacatcaaaattttattccagggagtccctagactcaacaacctacattggacatgcctctacgtacacacttcccgaagtgcttaccaggtcattatgtggtaaagaggacttatgtacgcttgacattacggatctattgttgaaagagattagtgctaagaaattggaggagtgtaggaagaagaatgcatctagtggtactccatctggtggtacaccttctaatgttccagaggagtgtgtaattagaaccaacttacagatggttatgaagaagaatgctcgtgccg

SEQ ID NO: 109 - DNA, 2559 bp, Babesia microti
  misc_feature (1)...(2559): n=A,T,C or G
ttcagaaattaatataattaattattataaatacaaaataattaattacaaaataacgtattattagccatttcagatttaaatacatatttttacatatattttaatttaaactttcaaattaatgtcatttttataaacattattataattatatactataattatcagtcatcaaataatatccaaagttatcctctacattatatcaatcactatggtatacaattatataacatattagcaacacataacaatcaaccttaacatatacataatattatgactaatcaataattatttattctcgcttgttataatgtagaatcattgtatactaatttattataaattattacaaaatacactcttttatttcattttatttcagttaaatttaatattttaatattatattcatctttctcatgttactttaatttatttccatttatatctcaatttctttatttaagagcaagatgttcgttcatgtatataataaatgtgnaaattttgnactatataataatatatacatctgaatttcatttaatttagaattaatattaatgcagtaaaaactagtgccatttgtccagggattattgaattagaaatcttggcgattgtggaaatatatgattttatgcttgaaataataccgctgtcaataacaataccaaggtccccaatggtgttattcatgacgtcaatagttttattaaattcttcaattgcaccattcacttcatttaaaatgtcattaaaattagtgccaaaattctgattattttctaatatcttggatatcaaaacattagcttctaattcgtgcttttcaaatgctttcttgatatcattaaattcgcttttatttgatataattggtatgtgcatattgtatatctcagttaaatatcccataattggtttaatattatcattcagcaggtttctaagtagttcagctttctttttcttaatttcatactcaaaattttcagcattttcaccttcaattgtaccaagagccttatagtatatttcatcaacaattgacttaatatcggtagagatagtggatatatcattaagcagtttaattatttcatttttaaccgtactattatttaaatcaatgtttgaaaaaatggcgttaaattccacaattttacttttgagctcaatttctttcttttcaaggtaatttttatgaccaccattcttctcatctgcttctagtatatcaatactatttttgcaggtatgaatcatagtgtccgtgatctgttcagaaaattttgcatacatttttttagcctcgttaaaagcgatcataagatcgtttaatgctcgtccaaaatcacttgaatattgattaacatcaccatttcctgcaaagacatttccacttgttaaaaagaatgtaataaaatgaattagtattggtctaactgacttcatccttatccacaattgttattgattaatatatatgtttattatagttataataacgttgtaataatgaaatacttgaattaatcttcagataataaatataagttcaagttataaattgatagacgttatattcttgttgattattatgtaataacatagttaatttattatatggggtcaaataattttgtctttatttgcctgcatacgtgatgtttatggtttatcgcttaattttattgtatattgtataaaaattgctctataataataataatataacagtaagatttgataatgataaatattgtatgataacataaataatactaattatttctacaaatatatgactatatcacataaataaatactatagtatagacatattttatataacatagatatattagtatattttatattattactttatcgttgtataatatactagtcatttgactttacttttattatggcatatcatttgtgtttatccttattcctaatacaatgttaatataaacgtatctccagtttataatgattgcaagtatagatgcttattaatataaaagtatcattggatatatttgtaatattgttaccaatatcacttttaactgacaatggtatgattcccttaataatccattgtttcatcacacaatatagatccatatgttaaataacaatttgattattataaatttagatataaataactttatttttataataattaattatattaatttcttaaatttcgaattattttaaataatattatacttcattaaattattttacataaatttccaaattcttatccttaatactatacacttttactcatttttgctcactacatttttgtttaccatattctgtattataagggggaaaggcaccatcacaaaaggtttcataatattcaatacgattatcgtcaacttgatgactaatactggtacattctaatttttcatttttccattcattattatccttcaatttgtagaaataatagcgggttgattttcctatagtagcagaaaattgtattaatattggttttttatgagaatcact

SEQ ID NO: 110 - DNA, 3141 bp, Babesia microti
  misc_feature (1)...(3141): n=A,T,C or G
acatgttgacttttggaaatatacgttttcataatataaatctcccaccattttcattgggcataattcactcgattacggtagaaaaggcgattaactctgaagattttgacggaatacaaacacttttacaagtgtctatcattgctagttacggtccatctggcgattacagtagttttgtgttcactccagttgtaacagcagacaccaacgttttttacaaattagagacggatttcaaacttgatgttgatgttattactaagacatcactagaattgcccacaagtgttcctggctttcactacaccgaaactatttaccaaggcacagaattgtcaaaatttagcaagcctcagtgcaaacttaacgatcctcctattacaacaggatcggggttgcaaataatacatgatggtttgaataattcgacaattataaccaacaaagaagttaatgtggatggaacagatttagttttttttgaattgctccctccatcggatggcattcccaccttgcgatcaaaattatttcccgtcctgaaatcaattccaatgatatctaccggggttaatgaattactgttggaagtactcgagaacccctctttccctagtgcaattagcaattacaccggactgacaggccgacttaacaaattacttacagttttagacggtattgttgatagcgccattagtgtcaagactacagaaactgtccctgacgacgcagaaacttctatttcttcattgaaatcattgataaaggcaatacgagataatattactaccactcgaaacgaagttaccaaagatgatgtttatgcattgaagaaggccctcacttgtctaacgacacacctaatatatcattcaagagtagatggtatatcattcgacatgctgggaacacaaaaaaataaatctagcccactaggcaagatcggaacgtctatggacgatattatagccatgttttcgaatcccaatatgtatcttgtgaaggtggcgtacttgcaagccattgaacacatttttctcatatcaaccaaatacaatgatatatttgattacaccattgattttagtaagcgtgaagctactgattctggatcatttaccgatatattgctcggaaacaaggtgaaggaatctttgtcatttattgagggtttgatttctgacataaaatctcactcattgaaagctggggttacaggaggtatatcaagttcatcattatttgatgaaatcttcgacgagttaaatttggatcaagcaacaattagaacccttgttgcaccattagaagaaattaaaaatgagcttaagactatttcctctcagaaaatagccgatgccacagtaaccccttctacccccaataccaatgtgaacatcaaaacaattatcagcaagattaagaaaattttgatgataagtgagactatttcatccacagctcttgcacgtttatctgcagtattaagcattcttggtagggggacttccacaaatgtcattccggaacgtctaactagtatcgttgttgatttgaaatcggcaactgttccacaggaagtggcgcttaagaatggagtttacaagttgaaggaccaatttaagctaacgcacaagatgatacctgtttttggcagcgtgcaactgcagattccagagaaatcaacagtcgtgcagataagtgtagtagagcatgaaaatgataccaaaatggcaatcatcacccttgatgatcattcgaaattgactttggaaagggtgattctttcagaaacccctactgttgttggtttaacccacaccacacaagatccactggatgtattgctatcaatatttgtcaagatggataatacaacggatgatggggttatggagggctatttagatctcgatttgaattccaagattggtacttttatttcggccatcgaactcattgacttgaccacccggtaaattcagcgagcgtccaccntccccaactaaaagtgttggctctgaagtttggcaccaagatcgttgatgtcgaggagacaggcaggacatttgttacatttgatgagaagttgaattcaatagaaataattaccttcgaaaatgatggcacttatgacatcaaaattttattccagggagtccctagacccaacaacctacatcggacatgcacctacagacatatttacgtcgccatggatcacgacccacatgcataacaagcgtcttgttgactttgaagttccatttgaagcaatttttgatgataaactcataagttattataccggtacggatgtcaacggcaagaataaggttcctgcagagcttaccaaggcaatatgcggcaaagaagacgtgtgtgagcttaacattaccggtttattgttgaaagatattagtgctaagaaattggaggagtgtaggaagaagaatgcatctagtggtactccatctggtggtacaccttctaatgttccagaggagtgtgtgattaaaagcaacttacagacggttatgaagaaggatgttactacaactttgaaatcggatgatgtcagcaattacagtgttgtatccattcacttttacattgataacgtgttcagacataatactgcttttggcagaattaagattggcaaccttgatctaccagcattttccattgggtttatccactcgatcttcgtcgagagggttctcatgggtgacaagagccttgccagtgttggcattataactaactacggtccaagtggagactatgagttgttgagatacatgcaagttgaggaagggaagaattatttcaaactcgtacaggggccagaaataacagctgattatattggatctgggttgactaaacacaagaggctgaccatgaatggcgcctccaccggttcaattggatttgaaaccaactacaaggaatcgatactcttcaatgagtttatgcgtccaaccaacaagatagtcaccctcttctatacggatagtgaaactgtcaatcttatcaagctgcactcattggagaatgtaaagcatggtgttacttattcaatttacggtgccttcccaattgaagaatcatctcctgaaagttcat

SEQ ID NO: 111 - DNA, 1134 bp, Babesia microti
acaggctcactctgactttggcaacaaacactagaggaggcgcaggtaccgatgccacaagtgtgagtatagcaaattcaatacctacttcagcagcaaccgccgctcaatcaacaacagctgctacatcaacaacagctgctacatcaacaacatctgctacatcaacaacatctgctacatcaacaacagctaccacatcaacaacaactgccacatcaacaacaactgccacatcaacaacagctaccacatcaacaacagctgccacatcaacaatttctccgtctctggagaccacacaagatgttgctgtcacaaatattgtgaatcttaacataaacgaaataggatttgttgatcaagttccagagggtctttcttctagttacgttttttctactgatggaatctttaccaaagttaccccagctacagggttttcaattggttgtgtaatatttggcaatcaattaattccacagtccatggatgttatcactaggaccgtttcatacaccactaaatatcctttgattgttgttaggattcaagataagacttcgagttctacttcaaccgtttactatgagcaatctggtttacaatctagcaaatttgttttgagggatgacccagaatttacatcccaactaacaagttctttctagccgggtggtggtagcacacacctttaatcccagcatttgggaggcagaggcaggtggatgtctgtgagttcgaggccagcctgatctatagcctgagttccaggacaggctccaaagctaccaagaaaccctgtcttggaaaagcaaaaaacaaacaaacaaaattcccagagctctcacggtcttattgtctctggtaataaataccaccactggcattaacctcactaatccccaggcatttccccctttctcttctgttttatttttgttttttccccagacacggtttccctgtttagtcatggctatactggaactaggtttgtacatgaggttggccttgaactcaaagattcacctgcctgggaattagaggtgttcaccaccatgctctgctggcatttcttaatccaattaatttgacattaaattaacaatcaaaacctcaggggaaaaaaac

SEQ ID NO: 112 - DNA, 3011 bp, Babesia microti
ctcgtgccgaatgtcatttatgatctaataatattgtattatctctaatattatggtataatagatactgtgaaaataaattcaactggagataaggaaaccatttgtatagatattttatacaaattattatgaaataatctaaataaatgacaaaaaatcgattatacaaatcacattaatgacaaacaaacttgtatacatatattgattaacattacaaaactaaattataatatttagattgataattgttataatacttaacaatattctactttttaatataattttttattcaataatatactctttcatattttgtactattttatataatcatatatattatataattatatatatttgataattgaatatatcaataatgatgatatacatgaatatgcatatataccccatataatgttattatatttagtgcttacattattaattataaatatatttaaataattaaataataatgaaaattaacatagacaatataatattaatcaatttgataatattattgaatcgtaatgtagtatattgtgtggataaaaatgatgtttcattatggaaatcaaaacctataacaactgtcagtaccactaatgatactattacaaataaatacactagtactgtaattaatgccaattttgctagctaccgtgaatttgaggatagggaacctttaacaataggatttgaatacatgatcgataaatcacaacaagataaattatcacatccaaataaaattgataaaatcaaaatttctgattatataattgaatttgatgacaatgctaaattaccaactggtagtgttaatgatatatccatcattacttgcaagcataataatccagtattaattagattctcatgtttaatagaaggatctatctgctattatttctacttattgaataatgatacaaataaatggaataatcacaaattaaaatatgataaaacatacaatgaacatactgacaataatggtattaattattataaaatcgattatagtgaatctacagaacctactaccgaatctactacctgtttttgttttcgcaaaaaaaatcataaatctgagcgtaaagaattagaaaattataaatatgagggtacagaattagcaagaatacattgtaataaagggaaatgtgtaaaattgggtgacattaagataaaggataagaatttggaaatttatgtgaaacagttaatgtctgtaaatactccagtaaattttgacaaccctacatcgattaatctaccaactgtcagtactaccaatgatactattacaaataaatacactggtactataattaatgccaatattgttgagtactgtgaatttgaggatgaacctttaacaatagggtttagatacactatagataaatcacaacaaaataaattatcacatccaaataaaattgataaaatcaaattttttgattatataattgaatttgatgatgatgttaaattaccaacaattggtactgtcaatattatatatatctatacttgcgagcataataatccagtattagttgaatttatagtttctatagaagaatcttactacttttacttctactcaatgaataatgatacaaataaatggaataatcataaaataaaatatgacaaaagatttaataaacatactgacatgaatggtattaattgttatgaatatgtacttcgtaaatgcagttcttatactcgtaaaaatgaatatgagcataaagaattagcaagaatacattgtaatgaagaaaaatgtgtaaatgtaaaggtacgataacattgagaaaaagaatttggaaatttatgtaaaataatttaacgaagtgtaatatgtaaaatagtttaatgaagtataatattatttaaaataattcaaaatttcagaaattaatataattaattattataaatacaaaataattaattacaaaataacgtattattagccatttcagatttaaatacatatttttacatatattttaatttaaactttcaaattaatgtcatttttataaacattattataattatatactataattatcagtcatcaaataatatccaaagttatcctctacattatatcaatcactatggtatacaattatataacatattagcaacacataacaataaaccttaacatatacataatattatgactaatcaataattatttattctcgcttgttataatgtagaatcattgtatactaatttattataaattattataaaatacattcttttatttcattttatttcagttaaatttaatattttaatattatattcatctttctcatgttactttaatttatttccatttatatctcaatttctttatttaagagcaagatgttcgttcatgtatataataaatgtgtaaattttgtactatataataatatatacatctgaatttcatttaatttagaattaatattaatgcagtaaaaactaatgccatttgtccagggattattgctttagaaatcttggcgattgtggaaatatatgattttatgcttgaaataataccgctgtcaataacaataccaaggtccccaatggtgttattcatgacgtcaatagttttattaaattcttcaattgcaccattcacttccccctaaacaaagactcaaaatcttcatctatactagccagctctatcgcaaaagcctccgcagctatcactaatgaaagtggaactaaaaaactactgtcaataaccaatactacaagcactggcgggaaaaccccttatgccttagaaaactgtatatctaaggatgaaaactcaaataatataccttcggtttcccctaacacgccattgatggatagggttactaaaacctgcctggaatattctggcgattttgaccctgactcatgcctatgggatgataccataggcaaagctcaaatccatattccta

SEQ ID NO: 113 - DNA, 1161 bp, Babesia microti
atgtgaatgcattgatcaaggagttgaacgctcatattaaacagagagcaacatctacaacaacaattattattgaaactaatgctaaagatgtggatgagttagtgaaaaaatttgcaacaattgcatcttttgatgataagttcaagaacgtattctttgataattctgttattgatgaaattgtcaaaacgttggaaaagatgaaggttgagtcagatactgtattacctagttgcaatggaatccagaccactgaaaactctagtactgacccatatacagtattatcaaaacttataaagaaaattaacgactccataatcagacctatgacttctcggctgatcaacaaatcctttccggagttgtgcaagttgtttataaaaatgcccgatgtcgactccaacaaatttatggctttggatgtggacataagcaacactcttgtaaacaggagagtcagatattctgatggcagatttaccattgtaagcactgggtccaattttagatacacattggcaccaactgccgctggtcatgatttgtctctcttctcccaattgccaatctcaatgattacggtcacatcgcctcaggagcaggcacttacatcttgcgtcagtcatggtaacgaattcagcatagtaagcactgcaggcaagacaacttacactacacaatctaagttgttgtcacttttcaagttatctgcggagacgttaagggattttaatgaagctagatttgcacttggtaacatgactgatagtgctaataaatctaaagctttggaggtctacaaatcgacactaactactatgaaatcaatatcagtcgaattggaaaagatttttggcatattaaaatcaactccgaatattacttttgaatcagttgtttctaaatacaaattgactggtgttaatacagttgatactgccaatgctgatgtgatcaacgagacaatgtttgacgatttgtccaaggcaatttcctcatacctatactccctcatatctataatttttccggaggatattaaaggtcaaggtacaagtgaaggtcaacaaacaagtggaggtcaggatacaaatgagacaattttctcatacctatactccctcatatctataatttttccggaggatattaaag

SEQ ID NO: 114 - DNA, 984 bp, Babesia microti; identical to SEQ ID NO: 107

SEQ ID NO: 115 - DNA, 1205 bp, Babesia microti
cacctggggcatggggtgtagtcacgtgggttcctgaatgacttggaatgcatagagggaactgcggttggggagggggacggactccgctggggcgcgtggcctcacctccgggtccggtggacacggtgacctgcgcgtaggtggcttccgcggcggccgctgggcctgagacaccgttgagcgcggccacgcgcacggtatagcgcgcgcccggccgcaggtgcagcagcgtggcggcgcgttctcgcaaccctgcctgacgcgggacgaaggccacgcgtggcccgcagggttggcatgcgcccgccggaccgtcgcggccgcagagcaggcacagcagcgagtaggtgacgtcggaacgaccgccggagtccgcaggcggcagccaccgcagtcgcagcgccaggggggagcggctcaagctgtactgcaggtcccgtggcgcggacggcggccctgcgacatggggtggcgctgagtggggccgggaggggacggaagcactagaggtgccggggcgcaggggcatgagcaccctgcatgggtagcagtatgagagccggcagtccttgatggggatgtggcgggggtgggggagcagccaggaactgagaggagggcatgaggaccatcgaggggactgggatggggacatggtcacctggaacagaggacttggaagacaggagggacattccagccaggcccagggagacacagagaatcttgaccccatctcagagaatcttgaccccatctccacccttctgggttccccagattctaaagcatatttatattttcataacaaagtatatatttgaacttccatgactagtaacttatctgcctgtgataatttgaatgtgaatggcccctgtaggttcatatatttgaacactcaggcacaagttttgaaactgtttgagaaggatttaggaataagtatgtacttgagggaagtgtgtcattagagggggattttgaggcttcaagcccacaccaggaccaggcttttctttgtctgatgccttaggatcagagtgtaaagctctcaattactgatccagcaccatgccaacctgctaccatgcttcccacaatgatggtcatgagtcaaccctcttaaactgtaaacaagcccccatttaaatgctttcttttacaaaaattgccatggccatggtaaccctttacacaat

SEQ ID NO: 116 - DNA, 1919 bp, Babesia microti
tagtcattagattatcatgacaccaataagctttttatcttgaagttgttttatatattaatacaaccatagcatcataaaagctacatttgttttttttatcttaacccatggtcatctagtctttttcctttattattcatcattgattgtccttaaatgctcaaagcatctgcccctttaaactacttctttctaaattagcatatactctatatggtcatacctattctgtgtaatcatcaggttccctgtgcaggggaaaggaggaacgctcaagcactgaggaatcatcccgctgtgtgataacgttgatggaagacaagtgatacagttagttgttcaaacaaataagcatatttaaggggaagaatagtgtcgtactaactaaaatctaatttgaccataatacgcacattagtttgtttgtgctcaatttttttaatgaatcaggcccccgatttatatttgtgaaagtccatgtgggagcgtaaggatgggatagtttatttacagtagcttctctggggaaaggaaagcaaagccccaactgtatagagttcattggagctgtcacctacgcccctgccttcctgtccctttagagtgcctcagtttgctgtgtggcaagagtctctccctgctcctgctctcctagccccctctgcctgcctcccccagttgatgcgagagtccactgttggagagttaactctaatcttacacctggggagagctactggaaattaattttccatgtaactgtctttgagttctagcaggctttagattttagaagtttttgtgtgtgtgtgtgtgtgtgtgtgtgtgtgtgtgtgtgtgtgtgtgtgtgtgtgtgtatgaagagggcacagtttttgcctttgattgcctctggaatgttccatttcttgtgattatcaaatagcagcatcagaggcagagtatggctcattttcccccgctatctccttaaagattataagatgatcctggtatgtatctgcttcgtgaacattgaagatctgggaacacaaaaaaataaatctagcccactaggcaagatcggaacgtctatggacgatattatagccatgttttcgaatcccaatatgtatcttgtgaaggtggcgtacttgcaagccattgaacacatttttctcatatcaaccaaatacaatgatatatttgattacaccattgattttagtaagcgtgaagctactgattctggatcatttaccgatatattgctcggaaacaaggtgaaggaatctttgtcatttattgagggtttgatttctgacataaaatctcactcattgaaagctggggttacaggaggtatatcaagttcatcattatttgatgaaatcttcgacgagttaaatttggatcaagcaacaattagaacccttgttgcaccattagaagaaattaaaaatgagcttaagactatttcctctcagaaaatagccgatgccacagtaaccccttctacccccaataccaatgtgaacatcaaaacaattatcagcaagattaagaaaattttgatgataagtgagactatttcatccacagctcttgcacgtttatctgcagtattaagcattcttggtagggggacttccacaaatgtcattccggaacgtctaactagtatcgttgttgatttgaaatcggcaactgttccacaggaagtggcgcttaagaatggagtttacaagttgaaggaccaatttaagctaacgcacaagatgatacctgtttttggcagcgtgcaactgcagattccagagaaatcaacagtcgtgcagataagtgtagtagagcatgaaaatgataccaaaatggcaatcatcacccttgatgatcattcgaaattgactttggaaagggtgattctttcagaaacc

SEQ ID NO: 117 - DNA, 4722 bp, Babesia microti
tgactggatatcgccaattgtattgtaacaggtggttgctatggaaatattaccaagctgcagcgctttatcgccgagtctcttccacctttcaatatttatgggttcaatttgcattttagacgcggattttctgggcgagtttacctcagatgaattatttggtgatttcaaggcacctttataaaatgtctcataagaatcaccattcaattgttccagacactaaatatttgtcaacttactaactcaacatcaccggtgctcaaagccaattcaaatttccttgatgcatttaccgttatggcaactgctagctgtatattacccatagcttcaactattacgcttatcctttcccgtaaatgggttggtatttcagcaaccatatcaagtactaatccccaattattctccaacattgcagtctaaatgaattgattggattacattcaacttcaaataagtttcagttaggttgtatgacagaacatttccatctcgatcaataagatatatcttgcttgtttcaatggaatagccgagaatgtacagtttacgatctagataagcgtgcggatcaggcacgccagttgtgtacaaatagagtctcagaccaactgttgtgtaaataaatgtatcacaaacccatactccactcacaatacgttctgtaatctaggttaaacaacaatttaacctcagtatccagctcaaatgttgatggtactccctcgtcatcctgaggcgtattggcagcgactgataccaaaaaggaattatgatcatattcttctagttacgttttttctactgatggaatctttaccaaagttaccccagctacagggttttcaattggttgtgtaatatttggcaatcaattaattccacagtccatggatgttatcactaggaccgtttcatacaccactaaatatcctttgattgttgttaggattcaagataagacttcgagttctacttcaaccgtttactatgagcaatctggtttacaatctagcaaatttgttttgagggatgacccagaatttattattcctcaaaatcgaagtagtacttatacagtcaatgacataacatataaatcatttgatatttctagtgccgatgataacgaatttttaaaaatatcattaagtgatgggagcatgttgtacaccaataatccagattccaaaatttacatcagcgaagttaaggttggtgagataacaataccaataaatataacatcacaatatacactgatcaaattatcatttaatggtgaattggttgagttgtatactacaggatgtttcggtgaacataatattaaaaagtttaggaaagtaggttctacctataatgatatatctaacgcttttgacattgtgccttggattccagctcattttgtcgtgactcagaaagtggatttttctataccttttgatttatttgaatcaaattatcacagcattttactaccagcaggtgtgaaccattctatccacattaatactgaaacagggaatgtggattcagttgtttttttcttgaatccactggccaagcacatgttgacttttggaaatatacgttttcataatataaatctcccaccattttcattgggcataattcactcgattacggtagaaaaggcgattaactctgaagattttgacggaatacaaacacttttacaagtgtctatcattgctagttacggtccatctggcgattacagtagttttgtgttcactccagttgtaacagcagacaccaacgttttttacaaattagagacggatttcaaacttgatgttgatgttattactaagacatcactagaattgcccacaagtgttcctggctttcactacaccgaaactatttaccaaggcacagaattgtcaaaatttagcaagcctcagtgcaaacttaacgatcctcctattacaacaggatcggggttgcaaataatacatgatggtttgaataattcgacaattataaccaacaaagaagttaatgtggatggaacagatttagttttttttgaattgctccctccatcggatggcattcccaccttgcgatcaaaattatttcccgtcctgaaatcaattccaatgatatctaccggggttaatgaattactgttggaagtactcgagaacccctctttccctagtgcaattagcaattacaccggactgacaggccgacttaacaaattacttacagttttagacggtattgttgatagcgccattagtgtcaagactacagaaactgtccctgacgacgcagaaacttctatttcttcattgaaatcattgataaaggcaatacgagataatattactaccactcgaaacgaagttaccaaagatgatgtttatgcattgaagaaggccctcacttgtctaacgacacacctaatatatcattcaagagtagatggtatatcattcgacatgctgggaacacaaaaaaataaatctagcccactaggcaagatcggaacgtctatggacgatattatagccatgttttcgaatcccaatatgtatcttgtgaaggtggcgtacttgcaagccattgaacacatttttctcatatcaaccaaatacaatgatatatttgattacaccattgattttagtaagcgtgaagctactgattctggatcatttaccgatatattgctcggaaacaaggtgaaggaatctttgtcatttattgagggtttgatttctgacataaaatctcactcattgaaagctggggttacaggaggtatatcaagttcatcattatttgatgaaatcttcgacgagttaaatttggatcaagcaacaattagaacccttgttgcaccattagaagaaattaaaaatgagcttaagactatttcctctcagaaaatagccgatgccacagtaaccccttctacccccaataccaatgtgaacatcaaaacaattatcagcaagattaagaaaattttgatgataagtgagactatttcatccacagctcttgcacgtttatctgcagtattaagcattcttggtagggggacttccacaaatgtcattccggaacgtctaactagtatcgttgttgatttgaaatcggcaactgttccacaggaagtggcgcttaagaatggagtttacaagttgaaggaccaatttaagctaacgcacaagatgatacctgtttttggcagcgtgcaactgcagattccagagaaatcaacagtcgtgcagataagtgtagtagagcatgaaaatgataccaaaatggcaatcatcacccttgatgatcattcgaaattgactttggaaagggtgattctttcagaaacccctactgttgttggtttaacccacaccacacaagatccactggatgtattgctatcaatatttgtcaagatggataatacaacggatgatggggttatggagggctatttagatctcgatttgaattccaagattggtaactttatttcggccatcgaactcactgacctgaccaacacggtaaaatcagcgagcgtccaccctccccaactaaaagtgttggctctgaagtttggcaacaagatcgttgatgtcgaggagacaggcaggacatttgttacatttgatgagaagttgaattcaatagaaataattaccttcgaaaatgatggcactatgacatcaaaattttattccagggagtccctagacccaacaacctacatcggacatgcacctacagacatatttacgtcgccatggatcacgacccacatgcataacaagcgtcttgttgactttgaagttccatttgaagcaatttttgatgataaactcataagttattataccggtacggatgtcaacggcaagaataaggttcctgcagagcttaccaaggcaatatgcggcaaagaagacgtgtgtgagcttaacattaccggtttattgttgaaagatattagtgctaagaaattggaggagtgtaggaagaagaatgcatctagtggtactccatctggtggtacaccttctaatgttccagaggagtgtgtgattaaaagcaacttacagacggttatgaagaaggatgttactacaactttgaaatcggatgatgtcagcaattacagtgttgtatccattcacttttacattgataacgtgttcagacataatactgcttttggcagaattaagattggcaaccttgatctaccagcattttccattgggtttatccactcgatcttcgtcgagagggttctcatgggtgacaagagccttgccagtgttggcattataactaactacggtccaagtggagactatgagttgttgagatacatgcaagttgaggaagggaagaattatttcaaactcgtacaggggccagaaataacagctgattatattggatctgggttgactaaacacaagaggctgaccatgaatggcgcctccaccggttcaattggatttgaaaccaactacaaggaatcgatactcttcaatgagtttatgcgtccaaccaacaagatagtcaccctcttctatacggatagtgaaactgtcaatcttatcaagctgcactcattggagaatgtaaagcatggtgttacttattcaatttacggtgccttcccaattgaagaatcatctcctgaaagttcat

SEQ ID NO: 118 - DNA, 2215 bp, Babesia microti
gttcctactttgtcatcattggtcaagttgttcagtgaagttatgctgagagtgaaggatgcgtcttccacagaggctaccatacgcatgttcctccgtttcaacgcatttataaaatttttgaatgaggagaaatccagaggtgacaaaagtgcgttgaatgatgagggattgatgaggtttatatcgatgaccagtggatttatcgatgaccttgaattagttttagatgagttatccaagcacagtttgcttataaataacgaaggtgccaagagcatgctatcctctctcatactaagcttccgttatattaatcacataagaaatttgatcaatggtatttaccttggattgaataacccatcatcgtccattggtgagacagcacaagaaacaactgaaccctccactcccactcccactcccagcacacagacaatcctgaaaccgaagggatccgagataaggggctatataataaaagttgatcaaacagctaatctcatcacattcatagatgcattgatcaaggagttgaacgttcatattaaacagacaacaacttcgtctgttgttggcactaaagaaactaatggcactaccagtggttctcctgaaagcaatcccggttccaccgattcaggttctattcaagctgaggtggcggaactattgaaaaaatttgcaacaattgcatcttttgacgagaagttcacgaacttacacattaataaaccttttgccgatgcacttattaaaaggttgaatgaaataaaggctgaactatcatctaatagtggaacccctcccaaattacccgatatatcatgtttaagactatcagaaattgtgcagaaacttaaccgtttaatcaaatttaatacttctcggctgatcaacaaatcctttccggagttgtgcaagttgtttataaaaatgcccgatgtcgactccaacaaatttatggctttggatgtggacataagcaacactcttgtaaacaggagagtcagatattctgatggtagatttaccattgtaagcactgggtccaattttagatacacattggcaccaactgccgctggtcatgatttgtctctcttctcccaattgccaatctcaatgattacggtcacatcgcctcaggagcaggcacttacatcttgcgtcagtcatggtaacgaattcagcatagtaagcactgcaggcaagacaacttacactacacaatctaagttgttgtcacttttcaagttatctgcggagacgttaagggattttaatgaagctagatttgcacttggtaacatgactgatagtgctaataaatctaaagctttggaggtctacaaatcgacactaactactatgaaatcaatatcagtcgaattggaaaagatttttggcatattaaaatcaactccgaatattacttttgaatcagttgtttctaaatacaaattgactggtgttaatacagttgatactgccaatgctgatgtgatcaacgagacaatgtttgacgatttgtccaaggcaatttcctcatacctatactccctcatatctataatttttccggaggatattaaaggtcaaggtacaagtgaaggtcaacaaacaagtgaaggtcaacaaacaagtgaaggtcaacaaacaagtggagatcaggatacaagtggaggtcaggatacaaatgagacaattttctcatacctatactccctcatatctataatttttccggaggatattaaaggtcaaggtacaagtgctcaattattggagtatagaactcaattggcatctctgagcaagatcaaatctctcagaaaaaaaataaaaagaaggctctgacattcgtatcctactttctgtagtttgtcttatgtaccaagtaccagtgtttcatagttctgtaggaatgaattcctattaaacatggtctccttttctcagagtttgttcatttagttgtttccactacttttcagttgctggaccgaagttcttatgggtaattacatttatcctcattacttctcaccctcaattttaatgttatatacacttttcataacacctagagtttcccctccctgcctttctccctttctccctacctcccctcaacccaccacacactgaggagtgaatactcctcagaaatgctaatgcctccctggcacattaagttgaggcaaggcca

SEQ ID NO: 119 - DNA, 3002 bp, Babesia microti
agtgattctcataaaaaaccaatattaatacaattttctgctactataggaaaatcaacccgctattatttctacaaattgaaggataataatgaatggaaaaatgaaaaattagaatgtaccagtattagtcatcaagttgacgataatcgtattgaatattatgaaaccttttgtgatggtgcctttcccccttataatacagaatatggtaaacaaaaatgtagtgagcaaaaatgagtaaaagtgtatagtattaaggataagaatttggaaatttatgtaaaataatttaatgaagtataatattatttaaaataattcgaaatttaagaaattaatataattaattattataaaaataaagttatttatatctaaatttataataatcaaattgttatttaacatatggatctatattgtgtgatgaaacaatggattattaagggaatcataccattgtcagttaaaagtgatattggtaacaatattacaaatatatccaatgatacttttatattaataagcatctatacttgcaatcattataaactggagatacgtttatattaacattgtattaggaataaggataaacacaaatgatatgccataataaaagtaaagtcaaatgactagtatattatacaacgataaagtaataatataaaatatactaatatatctatgttatataaaatatgtctatactatagtatttatttatgtgatatagtcatatatttgtagaaataattagtattatttatgttatcatacaatatttatcattatcaaatcttactgttatattattattattatagagcaatttttatacaatatacaataaaattaagcgataaaccataaacatcacgtatgcaggcaaataaagacaaaattatttgaccccatataataaattaactatgttattacataataatcaacaagaatataacgtctatcaatttataacttgaacttatatttattatctgaagattaattcaagtatttcattattacaacgttattataactataataaacatatatattaatcaataacaattgtggataaggatgaagtcagttagaccaatactaattcattttattacattctttttaacaagtggaaatgtctttgcaggaaatggtgatgttaatcaatattcaagtgattttggacgagcattaaacgatcttatgatcgcttttaacgaggctaaaaaaatgtatgcaaaattttctgaacagatcacggacactatgattcatacctgcaaaaatagtattgatatactagaagcagatgagaagaatggtggtcataaaaattaccttgaaaagaaagaaattgagctcaaaagtaaaattgtggaatttaacgccattttttcaaacattgatttaaataatagtacggttaaaaatgaaataattaaactgcttaatgatatatccactatctctaccgatattaagtcaattgttgatgaaatatactataaggctcttggtacaattgaaggtgaaaatgctgaaaattttgagtatgaaattaagaaaaagaaagctgaactacttagaaacctgctgaatgataatattaaaccaattatgggatatttaactgagatatacaatatgcacataccaattatatcaaataaaagcgaatttaatgatatcaagaaagcatttgaaaagcacgaattagaagctaatgttttgatatccaagatattagaaaataatcagaattttggcactaattttaatgacattttaaatgaagtgaatggtgcaattgaagaatttaataaaactattgacgtcatgaataacaccattggggaccttggtattgttattgacagcggtattatttcaagcataaaatcatatatttccacaatcgccaagatttctaattcaataatccctggacaaatggcactagtttttactgcattaatattaattctaaattaaatgaaattcagatgtatatattattatatagtacaaaatttacacatttattatatacatgaacgaacatcttgctcttaaataaagaaattgagatataaatggaaataaattaaagtaacatgagaaagatgaatataatattaaaatattaaatttaactgaaataaaatgaaataaaagagtgtattttgtaataatttataataaattagtatacaatgattctacattataacaagcgagaataaataattattgattagtcataatattatgtatatgttaaggtttattgttatgtgttgctaatatgttatataattgtataccatagtgattgatataatgtagaggataactttggatattatttgatgaccgataattatagtatataattataataatgtttataaaaatgacattaatttgaaagtttaaattaaaatatatgtaaaaatatgtatttaaatctgaaatggctaataatacgttattttgtaattaattattttgtatttataataattaattatattaatttctgaaattttgaattattttaaataatattatacttcattaaactattttacatattacacttcgttaaattattttagataaatttccaaatttttattcccaatgttatctacctttacatttacacatttttcttcattacaatgtattcttgctaattctttatgctcatattcatttttacaagtataagaactgcatttaccaagtacatattcataataattaatacccattcatgtcagtatgttcattaaatcttgtatcatattttattttatgattattccatttatttgtatcattattcattgaagtagaagtaattgtagtaagatccttctatagaaactataaattcaactaatactggattattatgcttgcaagtatagatatatataatattgacagtaccaattgttgttaatttaacactcgtgccg

SEQ ID NO: 120 - protein, 1312 aa, Babesia microti
SYSSSYVFSTDGIFTKVTPATGFSIGCVIFGNQLIPQSMDVITRTVSYTTKYPLIVVRIQDKTSSSTSTVYYEQSGLQSSKFVLRDDPEFIIPQNRSSTYTVNDITYKSFDISSADDNEFLKISLSDGSMLYTNNPDSKIYISEVKVGEITIPINITSQYTLIKLSFNGELVELYTTGCFGEHNIKKFRKVGSTYNDISNAFDIVPWIPAHFVVTQKVDFSIPFDLFESNYHSILLPAGVNHSIHINTETGNVDSVVFFLNPLAKHMLTFGNIRFHNINLPPFSLGIIHSITVEKAINSEDFDGIQTLLQVSIIASYGPSGDYSSFVFTPVVTADTNVFYKLETDFKLDVDVITKTSLELPTSVPGFHYTETIYQGTELSKFSKPQCKLNDPPITTGSGLQIIHDGLNNSTIITNKEVNVDGTDLVFFELLPPSDGIPTLRSKLFPVLKSIPMISTGVNELLLEVLENPSFPSAISNYTGLTGRLNKLLTVLDGIVDSAISVKTTETVPDDAETSISSLKSLIKAIRDNITTTRNEVTKDDVYALKKALTCLTTHLIYHSRVDGISFDMLGTQKNKSSPLGKIGTSMDDIIAMFSNPNMYLVKVAYLQAIEHIFLISTKYNDIFDYTIDFSKREATDSGSFTDILLGNKVKESLSFIEGLISDIKSHSLKAGVTGGISSSSLFDEIFDELNLDQATIRTLVAPLEEIKNELKTISSQKIADATVTPSTPNTNVNIKTIISKIKKILMISETISSTALARLSAVLSILGRGTSTNVIPERLTSIVVDLKSATVPQEVALKNGVYKLKDQFKLTHKMIPVFGSVQLQIPEKSTVVQISVVEHENDTKMAIITLDDHSKLTLERVILSETPTVVGLTHTTQDPLDVLLSIFVKMDNTTDDGVMEGYLDLDLNSKIGNFISAIELTDLTNTVKSASVHPPQLKVLALKFGNKIVDVEETGRTFVTFDEKLNSIEIITFENDGTMTSKFYSRESLDPTTYIGHAPTDIFTSPWITTHMHNKRLVDFEVPFEAIFDDKLISYYTGTDVNGKNKVPAELTKAICGKEDVCELNITGLLLKDISAKKLEECRKKNASSGTPSGGTPSNVPEECVIKSNLQTVMKKDVTTTLKSDDVSNYSVVSIHFYIDNVFRHNTAFGRIKIGNLDLPAFSIGFIHSIFVERVLMGDKSLASVGIITNYGPSGDYELLRYMQVEEGKNYFKLVQGPEITADYIGSGLTKHKRLTMNGASTGSIGFETNYKESILFNEFMRPTNKIVTLFYTDSETVNLIKLHSLENVKHGVTYSIYGAFPIEESSPESS

SEQ ID NO: 121 - protein, 309 aa, Babesia microti
QLWIRMKSVRPILIHFITFFLTSGNVFAGNGDVNQYSSDFGRALNDLMIAFNEAKKMYAKFSEQITDTMIHTCKNSIDILEADEKNGGHKNYLEKKEIELKSKIVEFNAIFSNIDLNNSTVKNEIIKLLNDISTISTDIKSIVDEIYYKALGTIEGENAENFEYEIKKKKAELLRNLLNDNIKPIMGYLTEIYNMHIPIISNKSEFNDIKKAFEKHELEANVLISKILENNQNFGTNFNDILNEVNGAIEEFNKTIDVMNNTIGDLGIVIDSGIISSIKSYISTIAKISNSIIPGQMALVFTALILILN

SEQ ID NO: 122 - protein, 222 aa, Babesia microti
RLTLTLATNTRGGAGTDATSVSIANSIPTSAATAAQSTTAATSTTAATSTTSATSTTSATSTTATTSTTTATSTTTATSTTATTSTTAATSTISPSLETTQDVAVTNIVNLNINEIGFVDQVPEGLSSSYVFSTDGIFTKVTPATGFSIGCVIFGNQLIPQSMDVITRTVSYTTKYPLIVVRIQDKTSSSTSTVYYEQSGLQSSKFVLRDDPEFTSQLTSSF

SEQ ID NO: 123 - protein, 452 aa, Babesia microti
IIMKINIDNIILINLIILLNRNVVYCVDKNDVSLWKSKPITTVSTTNDTITNKYTSTVINANFASYREFEDREPLTIGFEYMIDKSQQDKLSHPNKIDKIKISDYIIEFDDNAKLPTGSVNDISIITCKHNNPVLIRFSCLIEGSICYYFYLLNNDTNKWNNHKLKYDKTYNEHTDNNGINYYKIDYSESTEPTTESTTCFCFRKKNHKSERKELENYKYEGTELARIHCNKGKCVKLGDIKIKDKNLEIYVKQLMSVNTPVNFDNPTSINLPTVSTTNDTITNKYTGTIINANIVEYCEFEDEPLTIGFRYTIDKSQQNKLSHPNKIDKIKFFDYIIEFDDDVKLPTIGTVNIIYIYTCEHNNPVLVEFIVSIEESYYFYFYSMNNDTNKWNNHKIKYDKRFNKHTDMNGINCYEYVLRKCSSYTRKNEYEHKELARIHCNEEKCVNVKVR

SEQ ID NO: 124 - protein, 732 aa, Babesia microti
VPTLSSLVKLFSEVMLRVKDASSTEATIRMFLRFNAFIKFLNEEKSRGDKSALNDEGLMRFISMTSGFIDDLELVLDELSKHSLLINNEGAKSMLSSLILSFRYINHIRNLINGIYLGLNNPSSSIGETAQETTEPSTPTPTPSTQTILKPKGSEIRGYIIKVDQTANLITFIDALIKELNVHIKQTTTSSVVGTKETNGTTSGSPESNPGSTDSGSIQAEVAELLKKFATIASFDEKFTNLHINKPFADALIKRLNEIKAELSSNSGTPPKLPDISCLRLSEIVQKLNRLIKFNTSRLINKSFPELCKLFIKMPDVDSNKFMALDVDISNTLVNRRVRYSDGRFTIVSTGSNFRYTLAPTAAGHDLSLFSQLPISMITVTSPQEQALTSCVSHGNEFSIVSTAGKTTYTTQSKLLSLFKLSAETLRDFNEARFALGNMTDSANKSKALEVYKSTLTTMKSISVELEKIFGILKSTPNITFESVVSKYKLTGVNTVDTANADVINETMFDDLSKAISSYLYSLISIIFPEDIKGQGTSEGQQTSEGQQTSEGQQTSGDQDTSGGQDTNETIFSYLYSLISIIFPEDIKGQGTSAQLLEYRTQLASLSKIKSLRKKIKRRLHSYPTFCSLSYVPSTSVSFCRNEFLLNMVSFSQSLFILFPLLFSCWTEVLMGNYIYPHYFSPSILMLYTLFITPRVSPPCLSPFLPTSPQPTTHGVNTPQKCCLPGTLSGKA

SEQ ID NO: 125 - protein, 334 aa, Babesia microti
LSNSSIRGRVWLIFPRYLLKDYKMILVCICFVNIEDLGTQKNKSSPLGKIGTSMDDIIAMFSNPNMYLVKVAYLQAIEHIFLISTKYNDIFDYTIDFSKREATDSGSFTDILLGNKVKESLSFIEGLISDIKSHSLKAGVTGGISSSSLFDEIFDELNLDQATIRTLVAPLEEIKNELKTISSQKIADATVTPSTPNTNVNIKTIISKIKKILMISETISSTALARLSAVLSILGRGTSTNVIPERLTSIVVDLKSATVPQEVALKNGVYKLKDQFKLTHKMIPVFGSVQLQIPEKSTVVQISVVEHENDTKMAIITLDDHSKLTLERVILSET

SEQ ID NO: 126 - protein, 268 aa, Babesia microti
KYKYALESGEPRRVEMGSRFSEMGSRFSVSPWAWLECPSCLPSPLFQVTMSPSQSPRWSSCPPLSSWLLPHPRHIPIKDCRLSYCYPCRVLMPLRPGTSSASVPSRPHSAPPHVAGPPSAPRDLQYSLSRSPLALRLRWLPPADSGGRSDVTYSLLCLLCGRDGPAGACQPCGPRVAFVPRQAGLRERAATLLHLRPGARYTVRVAALNGVSGPAAAAEATYAQVTVSTGPGGEATRPSGVRPPPQPQFPLCIPSHSGTHVTTPHAPG

SEQ ID NO: 127 - protein, 386 aa, Babesia microti
VNALIKELNAHIKQRATSTTTIIIETNAKDVDELVKKFATIASFDDKFKNVFFDNSVIDEIVKTLEKMKVESDTVLPSCNGIQTTENSSTDPYTVLSKLIKKINDSIIRPMTSRLINKSFPELCKLFIKMPDVDSNKFMALDVDISNTLVNRRVRYSDGRFTIVSTGSNFRYTLAPTAAGHDLSLFSQLPISMITVTSPQEQALTSCVSHGNEFSIVSTAGKTTYTTQSKLLSLFKLSAETLRDFNEARFALGNMTDSANKSKALEVYKSTLTTMKSISVELEKIFGILKSTPNITFESVVSKYKLTGVNTVDTANADVINETMFDDLSKAISSYLYSLISIIFPEDIKGQGTSEGQQTSGGQDTNETIFSYLYSLISIIFPEDIK

SEQ ID NO: 128 - DNA, 1371 bp, Babesia microti
acataacactagggacttggcattgcatatctgtaaatataattgaaaccaaaataaaatattggtgagttccataggttgggttgttcacagtgacatttaaaagtgaaattcttgagagctggtttggaggttctattaggggagtgcggtacttgtataccttggactgaagaccagtcctcctctattccgggaaggtcgtcctcttcgaccaagcatgcagcttcaggatggacacacatggagtgttgagggaggaaagagatccccctaagccagatagatcaactaaatgaaccttggaaataaatggggtgacagatgtagcagcgagattgccctcacatactgaaaatgaaataattaaccaccattagttttccatctgatacctaggcactctctaatttaattcaacattctgaaaagtgtctttgaaagattggtggcaaccacctattatccctccaatgggtaggcaaagacaggtgaatcgaagtatgttgtagggaggctagtcttaatatagggttcaactacagggaagacttcatgctaagatgctatttcagataaaaaagaaatgtgtgttttttatctgacttcttattgtggcaccatagagcattgaaaagcaccgtatgctgttttgtggtatcagatcacattattttcacagttgaaaggcattataaaacaggttttgttgacactagactttaatcccagcatttgggaaacagaggcaggtggatcttggagattcgtgctagcctggtctacagtgggagtttaaggatagctgggatttcaatgagaaaccatgtccctggggtgaggggaaggaagaaaaaaagataatgtagtatgatacaggataaaatttaatagcatctttttaagaattatgagaacactctctgtcactgggttctatggattcagtttattgtttaactgtgggtgtttagctctgcttctatcagtcactggatgaaggtactagaatggcatataacactggaacacttaacagcttacttgaggctagtaagtgaagcacatgatgacaggtatggaggggttgtgcagaatggaactaagttctagagaatttgcagaaatggtcattataggacactgggtgaaaggtgcctctctgtacacttcctaaagctctgtccgttgcttggataatatgtgatctcttggacagtgatgtacctgaccttccctctgtctctgttctctttctccttcctttcccctcccctctgcttcccctcctctgccctcccctctcttctattcccttctctcctctctcattcattccccttgctcttctctcccttcttctctttcttt

SEQ ID NO: 129 - DNA, 2417 bp, Babesia microti
attttgtactgttcaaatgtgtaatatatttgtgaaagaagaaaataatttaagtcaagaggatgatgaaagggcagaagtaatacttgagataagcacttcacatcttacaattaaaactcttctgtgtctacctgcaaattcatgacagatgaaattaacttgttttctattcgttttctcctcttatttctgccagtattataatttcaggaaggaacatgcatcataaattacatgtaactttcatgttgcagtgatgctgttttctatttttgatctcatttgacagcagtaaagtcatacaaaaaataataaatacctctcatggagcttgccatttcctctgcatcttttttggggaagaagtggcctgaagagtagagcgttaagactcacaaagtcaagaactttcagatagaacccagccatcaattgcagccacaatgggtgctgaatccaacttcttgatttgtttttaaaggtattaggaataatattgattagcacttgtcaggttcacaatccaggacctaatacaagacacacctcaggtgaatttctgagagattatccatattagttaattgaggagggaaactctaccttaattgtccatgggaccagttcaacagctagagtactgaactgcataaaaaggagaaagtgacctgggcaggaacatgaccattctcttgcgcctcgctgcagaagaaatgtgatcagcctctttaaagtcctgtagcagtgactcccatgccacaatgaactgtagccaatttcatcatactgtcctagcttcctttctctctttataatactttgtactgatgtgaccaaattctgaaccctcaagtcaccaagaaacccattccaaggcaaaagcaaacagacttgtattatttaacaagttaatgccatctactccggtccttcatacgttcatcatggtgggtggaatgagaaggaccccaatgggccatgaggcagggaatttattgggcacagcaaggggagtgtctagggtcattgttagctgactcagagtgcagtgctttgcctcgaatcctgagcgcatttattcggctcttaaggtagccaaccatgcctggggggactgtcctgcttcaatagcagtaaaggccgaacaatcatggctgcattgtgactttgtgtgactctaatcttacatagaagagtaattcagagccccgtgttgctcttctggcccctgcgtgtgggagggtgccgacgtgatccagagccatgagacacccgtgccatcatccgtcccctcccgcgccacgccttcttcatgcctcgcttatggttctcctgtgtggtcgcaaacgtttgaaaacacgagcaacaagcaacaccttctgaaaattaacagcaaggttttcttaagaattcaccaagtgcaggctggagagagggcccagaggttaagagtactggctgctcttccagaggtcctgagttcaattcccagcacccacagggtggctcacaaccatctgtaatgagatcgtctgccctcttctggcccgcaggcagaacactgtatatataataagtaaataaatctctttttaaaaaagagtgaggtactgaagcaaccccataccaccgtgtgtcaatatgtgatttaaaaaaaaagaattaactaagtgcaggatactgtggccattgtctgcccctggaaggtcctgtgccccaggaaaagtctgcgtgctcctgtctccaggccatgcagagggctgaatcccccgtccgccccccacacgcacaacatatactcgttttgtctcctctgcagaatctagatttcacatacatattacacgcaagcagaaagttgaccgttagagaaattgctttctattaatttttaatttaagggagattgactacatcaatgaattaagaactgatacatcaatattgaattctggaagatgaactggggagaatgctccatgtggaaattgttcaccatataaccatggggatatgggctcagcctcagtactcttgtagcaaactggacacagcagcacaccttgagcccttgcacaggacatgcaaagacagaatgatgataccagtggctttggggccagccaagtctaccccattaagggaagactaacagtcggtaaccaggaactcaggttcagtacctgcctgtggctttataaaacttacttctagtttagatttccattttatgtttcattattccagatattctgtttttgatctatctgcttatgatttatatttcctaaattctcaacttgtaaatggcattagaaggatggaattgtacagtttcactttgtaattgttaagtcctatgctgtgtttttgcatgtttttgaagtgttttcagtaagtatttacttatttat

SEQ ID NO: 130 - DNA, 1333 bp, Babesia microti
aggtcacacatagaggagtgtggtcaattaaacactcaagcaccctatgtcttggtttgctctctattgctgtgataaacaccagagctaagcccaacttgaagttgtcacatggtctccacacaaatacacacacacacacacacacacacacacctatgtatgcacatgcaaccccacacacatacaaaaaaaaaagaacctctactctttaacagcaataaaaaatgaactaggtgaaaagaaaaccaaccttgcttcatcatttagtcatagaaaatgatactgtggttgtcatttactatcattaacctaaaataaatgtgtccctacctaagggtataaactgttatctggccttgtacagattttggatcttgaattcttttagtgggttgcccaatagcattttaaggtcccagaataaatagacaggatgaaatgggatgggctagagtagaatggaggctaatatcagaacaaatcagacagtgaggatatacttggctttacaagaatcctatttacacacacatgcacatgtactgtcagtatgtactgctacatcaacaacatctgctacatcaacaacagctaccacatcaacaacaactgccacatcaacaacaactgccacatcaacaacagctaccacatcaacaacagctgccacatcaacaatttctccgtctctggagaccacacaagatgttgctgtcacaaatattgtgaatcttaacataaacgaaataggatttgttgatcaagttccagagggtctttcttctagttacgttttttctactgatggaatctttaccaaagttaccccagctacagggttttcaattggttgtgtaatatttggcaatcaattaattccacagtccatggatgttatcactaggaccgtttcatacaccactaaatatcctttgattgttgttaggattcaagataagacttcgagttctacttcaaccgtttactatgagcaatctggtttacaatctagcaaatttgttttgagggatgacccagaatttattattcctcaaaatcgaagtagtacttatacagtcaatgacataacatataaatcatttgatatttctagtgccgatgataacgaatttttaaaaatatcattaagtgatgggagcatgttgtacaccaataatccagattccaaaatttacatcagcgaagttaaggttggtgagataacaataccaataaatataacatcacaatatacactgatcaaattatcatttaatggtgaattggttgagttgtatactacaggatgtttcggtgaacataatattaa

SEQ ID NO: 131 - DNA, 537 bp, Babesia microti; identical to SEQ ID NO: 108

SEQ ID NO: 132 - protein, 178 aa, Babesia microti
MEGYLDLDLNSKIGNFISAIELTNLTNTVKSASVHPPQLKVLALKFGNKIVDVEETGRTFVTFDEKLNSIEIITFENDGTMTSKFYSRESLDSTTYIGHASTYTLPEVLTRSLCGKEDLCTLDITDLLLKEISAKKLEECRKKNASSGTPSGGTPSNVPEECVIRTNLQMVMKKNARA

SEQ ID NO: 133 - protein, 292 aa, Babesia microti
SRMEANIRTNQTVRIYLALQESYLHTHAHVLSVCTATSTTSATSTTATTSTTTATSTTTATSTTATTSTTAATSTISPSLETTQDVAVTNIVNLNINEIGFVDQVPEGLSSSYVFSTDGIFTKVTPATGFSIGCVIFGNQLIPQSMDVITRTVSYTTKYPLIVVRIQDKTSSSTSTVYYEQSGLQSSKFVLRDDPEFIIPQNRSSTYTVNDITYKSFDISSADDNEFLKISLSDGSMLYTNNPDSKIYISEVKVGEITIPINITSQYTLIKLSFNGELVELYTTGCFGEHNI

SEQ ID NO: 134 - protein, 215 aa, Babesia microti
VQTFENDGTMTSKFYSRESLDPTTYIGHAPTDIFTSPWITTHMHNKRLVDFEVPFEAIFDDKLISYYTGTDVNGKNKVPAELTKAICGKEDVCELNITGLLLKDISAKKLEECRKKNASSGTPSGGTPSNVPEECVIKSNLQTVMKKDVTTTLKSDDVSNYSVVSIHFYIDNVFRHNTAFGRIKIGNLDLPAFSIGFIHSIFVERVLMGDKSLAS

SEQ ID NO: 135 - DNA, 2560 bp, Babesia microti
agtgattctcataaaaaaccaatattaatacaattttctgctactataggaaaatcaacccgctattatttctacaaattgaaggataataatgaatggaaaaatgaaaaattagaatgtaccagtattagtcatcaagttgacgataatcgtattgaatattatgaaaccttttgtgatggtgcctttcccccttataatacagaatatggtaaacaaaaatgtagtgagcaaaaatgagtaaaagtgtatagtattaaggataagaatttggaaatttatgtaaaataatttaatgaagtataatattatttaaaataattcgaaatttaagaaattaatataattaattattataaaaataaagttatttatatctaaatttataataatcaaattgttatttaacatatggatctatattgtgtgatgaaacaatggattattaagggaatcataccattgtcagttaaaagtgatattggtaacaatattacaaatatatccaatgatacttttatattaataagcatctatacttgcaatcattataaactggagatacgtttatattaacattgtattaggaataaggataaacacaaatgatatgccataataaaagtaaagtcaaatgactagtatattatacaacgataaagtaataatataaaatatactaatatatctatgttatataaaatatgtctatactatagtatttatttatgtgatatagtcatatatttgtagaaataattagtattatttatgttatcatacaatatttatcattatcaaatcttactgttatattattattattatagagcaatttttatacaatatacaataaaattaagcgataaaccataaacatcacgtatgcaggcaaataaagacaaaattatttgaccccatataataaattaactatgttattacataataatcaacaagaatataacgtctatcaatttataacttgaacttatatttattatctgaagattaattcaaagtatttcattattacaacgttattataactataataaacatatatattaatcaataacaattgtggataaggatgaagtcagttagaccaatactaattcattttattacattctttttaacaagtggaaatgtctttgcaggaaatggtgatgttaatcaatattcaagtgattttggacgagcattaaacgatcttatgatcgcttttaacgaggctaaaaaaatgtatgcaaaattttctgaacagatcacggacactatgtttcatacctacaaaaatagtattgatatactaaaagcagatgagaagaatggtggtcataaaaattaccttgaaaagaaagaaattgagctcaaaagtaaaactgtggaatttgacgtcattttttcaaacattgatttaaataatagtacggttaaaaatgaaataattaaactgcttaatgatatatccactatctctaccgatattaagtcaattgttgatgaaatatactataaggctcttggtacaattgaaggtgaaaatgctgaaaattttgagtatgaaattaagaaaaagaaagctgaactacttagaaacctgctgaatgataatattaaaccaattatgggatatttaactgagatatacaatatgcacataccaattatatcaaataaaagcgaatttaatgatatcaagaaagcatttgaaaagcacgaattagaagctaatgttttgatatccaagatattagaaaataatcagaattttggcactaattttaatgacattttaaatgaagtgaatggtgcaattgaagaatttaataaaactattgacgtcatgaataacaccattggggaccttggtattgttattgacagcggtattatttcaagcataaaatcacatatttccacaatcgccaagatttctaaagcaataatccctggacaaatggcattagtttttactgcattaatattaattctaaattaaatgaaattcagatgtatatattattatatagtacaaaatttacacatttattatatacatgaacgaacatcttgctcttaaataaagaaattgagatataaatggaaataaattaaagtaacatgagaaagatgaatataatattaaaatattaaatttaactgaaataaaatgaaataaaagagtgtattttgtaataatttataataaattagtatacaatgattctacattataacaagcgagaataaataattattgattagtcataatattatgtatatgttaaggttgattgttatgtgttgctaatatgttatataattgtataccatagtgattgatataatgtagaggataactttggatattatttgatgactgataattatagtatataattataataatgtttataaaaatgacattaatttgaaagtttaaattaaaatatatgtaaaaatatgtatttaaatctgaaatggctaataatacgttattttgtaattaattattttgtatttataataattaattatattaatttctgaa

SEQ ID NO: 136 - protein, 309 aa, Babesia microti
QLWIRMKSVRPILIHFITFFLTSGNVFAGNGDVNQYSSDFGRALNDLMIAFNEAKKMYAKFSEQITDTMFHTYKNSIDILKADEKNGGHKNYLEKKEIELKSKTVEFDVIFSNIDLNNSTVKNEIIKLLNDISTISTDIKSIVDEIYYKALGTIEGENAENFEYEIKKKKAELLRNLLNDNIKPIMGYLTEIYNMHIPIISNKSEFNDIKKAFEKHELEANVLISKILENNQNFGTNFNDILNEVNGAIEEFNKTIDVMNNTIGDLGIVIDSGIISSIKSHISTIAKISKAIIPGQMALVFTALILILN

SEQ ID NO: 137 - DNA, 24 bp, Artificial Sequence
  PCR primer
gatcctctggtggctccggttcta

SEQ ID NO: 138 - DNA, 24 bp, Artificial Sequence
  PCR primer
agcttagaaccggagccaccagag

SEQ ID NO: 139 - DNA, 29 bp, Artificial Sequence
  PCR primer
attccagaacccaatgcggattcagaatc

SEQ ID NO: 140 - DNA, 37 bp, Artificial Sequence
  PCR primer
cttgaattcatagaatcccaggaaagccttaaacatg

SEQ ID NO: 141 - DNA, 31 bp, Artificial Sequence
  PCR primer
ccgccgtagaattctcaatttacaataaatg

SEQ ID NO: 142 - DNA, 32 bp, Artificial Sequence
  PCR primer
ggttctaagcttacagatgatattaagaaggc

SEQ ID NO: 143 - DNA, 2034 bp, Babesia
atgcagcatcaccaccatcaccacattccagaacccaatgcggattcagaatctgtacatgttgaaatccaggaacatgataacatcaatccacaagacgcttgcgatagtgagccgctcgaacaaatggattctgataccagggtgttgcccgaaagtttggatgagggggtaccacaccaattctctagattagggcaccactcagacatggcatctgatataaatgatgaagaaccatcatttaaaatcggcgagaatgacataattcaaccaccctgggaagatacagctccataccattcaatagatgatgaagagcttgacaacttaatgagactaacggcgcaagaaacaagtgacgatcatgaagaagggaatggcaaactcaatacgaataaaagtgagaagactgaaagaaaatcgcatgatactcagacaccgcaagaaatatatgaagagcttgacaacttactgagactaacggcacaagaaatatatgaagagcgtaaagaagggcatggcaaacccaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagggcatgacaaaatcaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagagcatggcaatctcaataagaataaaagtgggaaggctggaataaaatcgcataatactcagacaccgctgaaaaaaaaagacttttgtaaagaagggtgtcatggttgcaataataagcccgaggataatgaaagagacccgtcgtcgcctgatgatgatggtggctgcgaatgcggcatgacgaatcactttgtctttgactacaagacaacactcttgttaaagagcctcaagactgaaacatccactcattattacattgccatggctgcaatttttactatttcattattcccatgcatgtttaaggctttcctgggatcctctggtggctccggttctaagcttacagatgatattaagaaggcatttgacgaatgcaaatctaatgctattatattgaagaaaaagatacttgacaatgatgaagattataagattaattttagggaaatggtgaatgaagtaacatgtgcaaacacaaaatttgaagccctaaatgatttgataatttccgactgtgagaaaaaaggtattaagataaacagagatgtgatttcaagctacaaattgcttctttccacaatcacctatattgttggagctggagttgaagctgtaactgttagtgtgtctgctacatctaatggaactgaatctggtggagctggtagtggaactggaactagtgtgtctgctacatctactttaactggtaatggtggaactgaatctggtggaacagctggaactactacgtctagtggaactgaagctggtggaactagtggaactactacgtctagtggagctgctagtggtaaagctggaactggaacagctggaactactacgtctagtgaaggtgctggtagtgataaagctggaactggaactagtggaactactacgtctagtggaactggtgctggtggagctggtagtggtggacctagtggacatgcttctaatgcaaaaattcctggaataatgacactaactctatttgcattattaacatttattgtaaattga

SEQ ID NO: 144 - protein, 677 aa, Babesia
MQHHHHHHIPEPNADSESVHVEIQEHDNINPQDACDSEPLEQMDSDTRVLPESLDEGVPHQFSRLGHHSDMASDINDEEPSFKIGENDIIQPPWEDTAPYHSIDDEELDNLMRLTAQETSDDHEEGNGKLNTNKSEKTERKSHDTQTPQEIYEELDNLLRLTAQEIYEERKEGHGKPNTNKSEKAERKSHDTQTTQEICEECEEGHDKINKNKSGNAGIKSYDTQTTQEICEECEEGHDKINKNKSGNAGIKSYDTQTPQETSDAHEEGHDKINTNKSEKAERKSHDTQTTQEICEECEEGHDKINKNKSGNAGIKSYDTQTPQETSDAHEEEHGNLNKNKSGKAGIKSHNTQTPLKKKDFCKEGCHGCNNKPEDNERDPSSPDDDGGCECGMTNHFVFDYKTTLLLKSLKTETSTHYYIAMAAIFTISLFPCMFKAFLGSSGGSGSKLTDDIKKAFDECKSNAIILKKKILDNDEDYKINFREMVNEVTCANTKFEALNDLIISDCEKKGIKINRDVISSYKLLLSTITYIVGAGVEAVTVSVSATSNGTESGGAGSGTGTSVSATSTLTGNGGTESGGTAGTTTSSGTEAGGTSGTTTSSGAASGKAGTGTAGTTTSSEGAGSDKAGTGTSGTTTSSGTGAGGAGSGGPSGHASNAKIPGIMTLTLFALLTFIVN

SEQ ID NO: 145 - DNA, 26 bp, Artificial Sequence
  Primer
gacgagttaaatttggatcaagcaac

SEQ ID NO: 146 - DNA, 30 bp, Artificial Sequence
  Primer
cataactcgagtcatcaatgaactttcagg

SEQ ID NO: 147 - DNA, 30 bp, Artificial Sequence
  Primer
gatgttatcactaggaccgtttcatacacc

SEQ ID NO: 148 - DNA, 30 bp, Artificial Sequence
  Primer
cataagaattcatcagtgcttggccagtgg

SEQ ID NO: 149 - DNA, 31 bp, Artificial Sequence
  Primer
gtgaatgcattgatcaaggagttgaacgctc

SEQ ID NO: 150 - DNA, 28 bp, Artificial Sequence
  Primer
cccctcgaggtcgacggtatcgataagc

SEQ ID NO: 151 - DNA, 30 bp, Artificial Sequence
  Primer
ctgagagtgaaggatgcgtcttccacagag

SEQ ID NO: 152 - DNA, 30 bp, Artificial Sequence
  Primer
ctcgaactcgagctacagaaagtaggatac

SEQ ID NO: 153 - DNA, 29 bp, Artificial Sequence
  Primer
cattttattacattctttttaacaagtgg

SEQ ID NO: 154 - DNA, 37 bp, Artificial Sequence
  Primer
ccgagaattcattaatttagaattaatattaatgcag

SEQ ID NO: 155 - DNA, 27 bp, Artificial Sequence
  Primer
cgcctcactctgactttggcaacaaac

SEQ ID NO: 156 - DNA, 36 bp, Artificial Sequence
  Primer
cttgtagaattcactagaaagaacttgttagttggg

SEQ ID NO: 157 - DNA, 30 bp, Artificial Sequence
  Primer
gagggctatttagatctcgatttgaattcc

SEQ ID NO: 158 - DNA, 30 bp, Artificial Sequence
  Primer
caatactcgagttatcaggcacgagcattc

SEQ ID NO: 159 - DNA, 39 bp, Artificial Sequence
  Primer
ggttctcgtttctctgagatggggtcaagattctctgtg

SEQ ID NO: 160 - DNA, 36 bp, Artificial Sequence
  Primer
ccaatagaattcatcaacctggggcatggggtgtag

SEQ ID NO: 161 - DNA, 27 bp, Artificial Sequence
  Primer
cgcctcactctgactttggcaacaaac

SEQ ID NO: 162 - DNA, 36 bp, Artificial Sequence; identical to SEQ ID NO: 156
  Primer

SEQ ID NO: 163 - DNA, 1906 bp, Babesia microti
atgcagcatcaccaccatcaccacgacgagttaaatttggatcaagcaacaattagaacccttgttgcaccattagaagaaattaaaaatgagcttaagactatttcctctcagaaaatagccgatgccacagtaaccccttctacccccaataccaatgtgaacatcaaaacaattatcagcaagattaagaaaattttgatgataagtgagactatttcatccacagctcttgcacgtttatctgcagtattaagcattcttggtagggggacttccacaaatgtcattccggaacgtctaactagtatcgttgttgatttgaaatcggcaactgttccacaggaagtggcgcttaagaatggagtttacaagttgaaggaccaatttaagctaacgcacaagatgatacctgtttttggcagcgtgcaactgcagattccagagaaatcaacagtcgtgcagataagtgtagtagagcatgaaaatgataccaaaatggcaatcatcacccttgatgatcattcgaaattgactttggaaagggtgattctttcagaaacccctactgttgttggtttaacccacaccacacaagatccactggatgtattgctatcaatatttgtcaagatggataatacaacggatgatggggttatggagggctatttagatctcgatttgaattccaagattggtaactttatttcggccatcgaactcactgacctgaccaacacggtaaaatcagcgagcgtccaccctccccaactaaaagtgttggctctgaagtttggcaacaagatcgttgatgtcgaggagacaggcaggacatttgttacatttgatgagaagttgaattcaatagaaataattaccttcgaaaatgatggcactatgacatcaaaattttattccagggagtccctagacccaacaacctacatcggacatgcacctacagacatatttacgtcgccatggatcacgacccacatgcataacaagcgtcttgttgactttgaagttccatttgaagcaatttttgatgataaactcataagttattataccggtacggatgtcaacggcaagaataaggttcctgcagagcttaccaaggcaatatgcggcaaagaagacgtgtgtgagcttaacattaccggtttattgttgaaagatattagtgctaagaaattggaggagtgtaggaagaagaatgcatctagtggtactccatctggtggtacaccttctaatgttccagaggagtgtgtgattaaaagcaacttacagacggttatgaagaaggatgttactacaactttgaaatcggatgatgtcagcaattacagtgttgtatccattcacttttacattgataacgtgttcagacataatactgcttttggcagaattaagattggcaaccttgatctaccagcattttccattgggtttatccactcgatcttcgtcgagagggttctcatgggtgacaagagccttgccagtgttggcattataactaactacggtccaagtggagactatgagttgttgagatacatgcaagttgaggaagggaagaattatttcaaactcgtacaggggccagaaataacagctgattatattggatctgggttgactaaacacaagaggctgaccatgaatggcgcctccaccggttcaattggatttgaaaccaactacaaggaatcgatactcttcaatgagtttatgcgtccaaccaacaagatagtcaccctcttctatacggatagtgaaactgtcaatcttatcaagctgcactcattggagaatgtaaagcatggtgttacttattcaatttacggtgccttcccaattgaagaatcatctcctgaaagttcattgatga

SEQ ID NO: 164 - DNA, 711 bp, Babesia microti
atgcagcatcaccaccatcaccacgatgttatcactaggaccgtttcatacaccactaaatatcctttgattgttgttaggattcaagataagacttcgagttctacttcaaccgtttactatgagcaatctggtttacaatctagcaaatttgttttgagggatgacccagaatttattattcctcaaaatcgaagtagtacttatacagtcaatgacataacatataaatcatttgatatttctagtgccgatgataacgaatttttaaaaatatcattaagtgatgggagcatgttgtacaccaataatccagattccaaaatttacatcagcgaagttaaggttggtgagataacaataccaataaatataacatcacaatatacactgatcaaattatcatttaatggtgaattggttgagttgtatactacaggatgtttcggtgaacataatattaaaaagtttaggaaagtaggttctacctataatgatatatctaacgcttttgacattgtgccttggattccagctcattttgtcgtgactcagaaagtggatttttctataccttttgatttatttgaatcaaattatcacagcattttactaccagcaggtgtgaaccattctatccacattaatactgaaacagggaatgtggattcagttgtttttttcttgaatccactggccaagcactgatga

SEQ ID NO: 165 - DNA, 1248 bp, Babesia microti
atgcagcatcaccaccatcaccacgtgaatgcattgatcaaggagttgaacgctcatattaaacagagagcaacatctacaacaacaattattattgaaactaatgctaaagatgtggatgagttagtgaaaaaatttgcaacaattgcatcttttgatgataagttcaagaacgtattctttgataattctgttattgatgaaattgtcaaaacgttggaaaagatgaaggttgagtcagatactgtattacctagttgcaatggaatccagaccactgaaaactctagtactgacccatatacagtattatcaaaacttataaagaaaattaacgactccataatcagacctatgacttctcggctgatcaacaaatcctttccggagttgtgcaagttgtttataaaaatgcccgatgtcgactccaacaaatttatggctttggatgtggacataagcaacactcttgtaaacaggagagtcagatattctgatggcagatttaccattgtaagcactgggtccaattttagatacacattggcaccaactgccgctggtcatgatttgtctctcttctcccaattgccaatctcaatgattacggtcacatcgcctcaggagcaggcacttacatcttgcgtcagtcatggtaacgaattcagcatagtaagcactgcaggcaagacaacttacactacacaatctaagttgttgtcacttttcaagttatctgcggagacgttaagggattttaatgaagctagatttgcacttggtaacatgactgatagtgctaataaatctaaagctttggaggtctacaaatcgacactaactactatgaaatcaatatcagtcgaattggaaaagatttttggcatattaaaatcaactccgaatattacttttgaatcagttgtttctaaatacaaattgactggtgttaatacagttgatactgccaatgctgatgtgatcaacgagacaatgtttgacgatttgtccaaggcaatttcctcatacctatactccctcatatctataatttttccggaggatattaaaggtcaaggtacaagtgaaggtcaacaaacaagtggaggtcaggatacaaatgagacaattttctcatacctatactccctcatatctataatttttccggaggatattaaaggtgccgaattcgatatcaagcttatcgataccgtcgacctcgagcaccaccaccaccaccactga

SEQ ID NO: 166 - DNA, 1842 bp, Babesia microti
atgcagcatcaccaccatcaccacctgagagtgaaggatgcgtcttccacagaggctaccatacgcatgttcctccgtttcaacgcatttataaaatttttgaatgaggagaaatccagaggtgacaaaagtgcgttgaatgatgagggattgatgaggtttatatcgatgaccagtggatttatcgatgaccttgaattagttttagatgagttatccaagcacagtttgcttataaataacgaaggtgccaagagcatgctatcctctctcatactaagcttccgttatattaatcacataagaaatttgatcaatggtatttaccttggattgaataacccatcatcgtccattggtgagacagcacaagaaacaactgaaccctccactcccactcccactcccagcacacagacaatcctgaaaccgaagggatccgagataaggggctatataataaaagttgatcaaacagctaatctcatcacattcatagatgcattgatcaaggagttgaacgttcatattaaacagacaacaacttcgtctgttgttggcactaaagaaactaatggcactaccagtggttctcctgaaagcaatcccggttccaccgattcaggttctattcaagctgaggtggcggaactattgaaaaaatttgcaacaattgcatcttttgacgagaagttcacgaacttacacattaataaaccttttgccgatgcacttattaaaaggttgaatgaaataaaggctgaactatcatctaatagtggaacccctcccaaattacccgatatatcatgtttaagactatcagaaattgtgcagaaacttaaccgtttaatcaaatttaatacttctcggctgatcaacaaatcctttccggagttgtgcaagttgtttataaaaatgcccgatgtcgactccaacaaatttatggctttggatgtggacataagcaacactcttgtaaacaggagagtcagatattctgatggtagatttaccattgtaagcactgggtccaattttagatacacattggcaccaactgccgctggtcatgatttgtctctcttctcccaattgccaatctcaatgattacggtcacatcgcctcaggagcaggcacttacatcttgcgtcagtcatggtaacgaattcagcatagtaagcactgcaggcaagacaacttacactacacaatctaagttgttgtcacttttcaagttatctgcggagacgttaagggattttaatgaagctagatttgcacttggtaacatgactgatagtgctaataaatctaaagctttggaggtctacaaatcgacactaactactatgaaatcaatatcagtcgaattggaaaagatttttggcatattaaaatcaactccgaatattacttttgaatcagttgtttctaaatacaaattgactggtgttaatacagttgatactgccaatgctgatgtgatcaacgagacaatgtttgacgatttgtccaaggcaatttcctcatacctatactccctcatatctataatttttccggaggatattaaaggtcaaggtacaagtgaaggtcaacaaacaagtgaaggtcaacaaacaagtgaaggtcaacaaacaagtggagatcaggatacaagtggaggtcaggatacaaatgagacaattttctcatacctatactccctcatatctataatttttccggaggatattaaaggtcaaggtacaagtgctcaattattggagtatagaactcaattggcatctctgagcaagatcaaatctctcagaaaaaaaataaaaagaaggctctga

SEQ ID NO: 167 - DNA, 918 bp, Babesia microti
atgcagcatcaccaccatcaccaccattttattacattctttttaacaagtggaaatgtctttgcaggaaatggtgatgttaatcaatattcaagtgattttggacgagcattaaacgatcttatgatcgcttttaacgaggctaaaaaaatgtatgcaaaattttctgaacagatcacggacactatgattcatacctgcaaaaatagtattgatatactagaagcagatgagaagaatggtggtcataaaaattaccttgaaaagaaagaaattgagctcaaaagtaaaattgtggaatttaacgccattttttcaaacattgatttaaataatagtacggttaaaaatgaaataattaaactgcttaatgatatatccactatctctaccgatattaagtcaattgttgatgaaatatactataaggctcttggtacaattgaaggtgaaaatgctgaaaattttgagtatgaaattaagaaaaagaaagctgaactacttagaaacctgctgaatgataatattaaaccaattatgggatatttaactgagatatacaatatgcacataccaattatatcaaataaaagcgaatttaatgatatcaagaaagcatttgaaaagcacgaattagaagctaatgttttgatatccaagatattagaaaataatcagaattttggcactaattttaatgacattttaaatgaagtgaatggtgcaattgaagaatttaataaaactattgacgtcatgaataacaccattggggaccttggtattgttattgacagcggtattatttcaagcataaaatcatatatttccacaatcgccaagatttctaattcaataatccctggacaaatggcactagtttttactgcattaatattaattctaaattaatgatga

SEQ ID NO: 168 - DNA, 696 bp, Babesia microti
atgcagcatcaccaccatcaccaccgcctcactctgactttggcaacaaacactagaggaggcgcaggtaccgatgccacaagtgtgagtatagcaaattcaatacctacttcagcagcaaccgccgctcaatcaacaacagctgctacatcaacaacagctgctacatcaacaacatctgctacatcaacaacatctgctacatcaacaacagctaccacatcaacaacaactgccacatcaacaacaactgccacatcaacaacagctaccacatcaacaacagctgccacatcaacaatttctccgtctctggagaccacacaagatgttgctgtcacaaatattgtgaatcttaacataaacgaaataggatttgttgatcaagttccagagggtctttcttctagttacgttttttctactgatggaatctttaccaaagttaccccagctacagggttttcaattggttgtgtaatatttggcaatcaattaattccacagtccatggatgttatcactaggaccgtttcatacaccactaaatatcctttgattgttgttaggattcaagataagacttcgagttctacttcaaccgtttactatgagcaatctggtttacaatctagcaaatttgttttgagggatgacccagaatttacatcccaactaacaagttctttctagtga

SEQ ID NO: 169 - DNA, 786 bp, Babesia microti
atgcagcatcaccaccatcaccacggttctcgtttctctgagatggggtcaagattctctgtgtctccctgggcctggctggaatgtccctcctgtcttccaagtcctctgttccaggtgaccatgtccccatcccagtcccctcgatggtcctcatgccctcctctcagttcctggctgctcccccacccccgccacatccccatcaaggactgccggctctcatactgctacccatgcagggtgctcatgcccctgcgccccggcacctctagtgcttccgtcccctcccggccccactcagcgccaccccatgtcgcagggccgccgtccgcgccacgggacctgcagtacagcttgagccgctcccccctggcgctgcgactgcggtggctgccgcctgcggactccggcggtcgttccgacgtcacctactcgctgctgtgcctgctctgcggccgcgacggtccggcgggcgcatgccaaccctgcgggccacgcgtggccttcgtcccgcgtcaggcagggttgcgagaacgcgccgccacgctgctgcacctgcggccgggcgcgcgctataccgtgcgcgtggccgcgctcaacggtgtctcaggcccagcggccgccgcggaagccacctacgcgcaggtcaccgtgtccaccggacccggaggtgaggccacgcgccccagcggagtccgtccccctccccaaccgcagttccctctatgcattccaagtcattcaggaacccacgtgactacaccccatgccccaggttgatga

SEQ ID NO: 170 - DNA, 561 bp, Babesia microti
atgcagcatcaccaccatcaccacgagggctatttagatctcgatttgaattccaagattggtaactttatttcagccatcgaactcactaacctgaccaacacggtaaaatcagcgagcgtccaccctccccaactaaaagtgttggctctgaagtttggcaacaagatcgttgatgtcgaggagacaggcaggacatttgttacatttgatgagaagttgaattcaatagaaataattaccttcgaaaatgatggcactatgacatcaaaattttattccagggagtccctagactcaacaacctacattggacatgcctctacgtacacacttcccgaagtgcttaccaggtcattatgtggtaaagaggacttatgtacgcttgacattacggatctattgttgaaagagattagtgctaagaaattggaggagtgtaggaagaagaatgcatctagtggtactccatctggtggtacaccttctaatgttccagaggagtgtgtaattagaaccaacttacagatggttatgaagaagaatgctcgtgcctgataa

SEQ ID NO: 171 - DNA, 897 bp, Babesia microti
atgcagcatcaccaccatcaccacgaggctaatatcagaacaaatcagacagtgaggatatacttggctttacaagaatcctatttacacacacatgcacatgtactgtcagtatgtactgctacatcaacaacatctgctacatcaacaacagctaccacatcaacaacaactgccacatcaacaacaactgccacatcaacaacagctaccacatcaacaacagctgccacatcaacaatttctccgtctctggagaccacacaagatgttgctgtcacaaatattgtgaatcttaacataaacgaaataggatttgttgatcaagttccagagggtctttcttctagttacgttttttctactgatggaatctttaccaaagttaccccagctacagggttttcaattggttgtgtaatatttggcaatcaattaattccacagtccatggatgttatcactaggaccgtttcatacaccactaaatatcctttgattgttgttaggattcaagataagacttcgagttctacttcaaccgtttactatgagcaatctggtttacaatctagcaaatttgttttgagggatgacccagaatttattattcctcaaaatcgaagtagtacttatacagtcaatgacataacatataaatcatttgatatttctagtgccgatgataacgaatttttaaaaatatcattaagtgatgggagcatgttgtacaccaataatccagattccaaaatttacatcagcgaagttaaggttggtgagataacaataccaataaatataacatcacaatatacactgatcaaattatcatttaatggtgaattggttgagttgtatactacaggatgtttcggtgaacataatatttgatga

SEQ ID NO: 172 - protein, 635 aa, Babesia microti
MQHHHHHHDELNLDQATIRTLVAPLEEIKNELKTISSQKIADATVTPSTPNTNVNIKTIISKIKKILMISETISSTALARLSAVLSILGRGTSTNVIPERLTSIVVDLKSATVPQEVALKNGVYKLKDQFKLTHKMIPVFGSVQLQIPEKSTVVQISVVEHENDTKMAIITLDDHSKLTLERVILSETPTVVGLTHTTQDPLDVLLSIFVKMDNTTDDGVMEGYLDLDLNSKIGNFISAIELTDLTNTVKSASVHPPQLKVLALKFGNKIVDVEETGRTFVTFDEKLNSIEIITFENDGTMTSKFYSRESLDPTTYIGHAPTDIFTSPWITTHMHNKRLVDFEVPFEAIFDDKLISYYTGTDVNGKNKVPAELTKAICGKEDVCELNITGLLLKDISAKKLEECRKKNASSGTPSGGTPSNVPEECVIKSNLQTVMKKDVTTTLKSDDVSNYSVVSIHFYIDNVFRHNTAFGRIKIGNLDLPAFSIGFIHSIFVERVLMGDKSLASVGIITNYGPSGDYELLRYMQVEEGKNYFKLVQGPEITADYIGSGLTKHKRLTMNGASTGSIGFETNYKESILFNEFMRPTNKIVTLFYTDSETVNLIKLHSLENVKHGVTYSIYGAFPIEESSPESSLM

SEQ ID NO: 173 - protein, 235 aa, Babesia microti
MQHHHHHHDVITRTVSYTTKYPLIVVRIQDKTSSSTSTVYYEQSGLQSSKFVLRDDPEFIIPQNRSSTYTVNDITYKSFDISSADDNEFLKISLSDGSMLYTNNPDSKIYISEVKVGEITIPINITSQYTLIKLSFNGELVELYTTGCFGEHNIKKFRKVGSTYNDISNAFDIVPWIPAHFVVTQKVDFSIPFDLFESNYHSILLPAGVNHSIHINTETGNVDSVVFFLNPLAKH

SEQ ID NO: 174 - protein, 415 aa, Babesia microti
MQHHHHHHVNALIKELNAHIKQRATSTTTIIIETNAKDVDELVKKFATIASFDDKFKNVFFDNSVIDEIVKTLEKMKVESDTVLPSCNGIQTTENSSTDPYTVLSKLIKKINDSIIRPMTSRLINKSFPELCKLFIKMPDVDSNKFMALDVDISNTLVNRRVRYSDGRFTIVSTGSNFRYTLAPTAAGHDLSLFSQLPISMITVTSPQEQALTSCVSHGNEFSIVSTAGKTTYTTQSKLLSLFKLSAETLRDFNEARFALGNMTDSANKSKALEVYKSTLTTMKSISVELEKIFGILKSTPNITFESVVSKYKLTGVNTVDTANADVINETMFDDLSKAISSYLYSLISIIFPEDIKGQGTSEGQQTSGGQDTNETIFSYLYSLISIIFPEDIKGAEFDIKLIDTVDLEHHHHHH

SEQ ID NO: 175 - protein, 613 aa, Babesia microti
MQHHHHHHLRVKDASSTEATIRMFLRFNAFIKFLNEEKSRGDKSALNDEGLMRFISMTSGFIDDLELVLDELSKHSLLINNEGAKSMLSSLILSFRYINHIRNLINGIYLGLNNPSSSIGETAQETTEPSTPTPTPSTQTILKPKGSEIRGYIIKVDQTANLITFIDALIKELNVHIKQTTTSSVVGTKETNGTTSGSPESNPGSTDSGSIQAEVAELLKKFATIASFDEKFTNLHINKPFADALIKRLNEIKAELSSNSGTPPKLPDISCLRLSEIVQKLNRLIKFNTSRLINKSFPELCKLFIKMPDVDSNKFMALDVDISNTLVNRRVRYSDGRFTIVSTGSNFRYTLAPTAAGHDLSLFSQLPISMITVTSPQEQALTSCVSHGNEFSIVSTAGKTTYTTQSKLLSLFKLSAETLRDFNEARFALGNMTDSANKSKALEVYKSTLTTMKSISVELEKIFGILKSTPNITFESVVSKYKLTGVNTVDTANADVINETMFDDLSKAISSYLYSLISIIFPEDIKGQGTSEGQQTSEGQQTSEGQQTSGDQDTSGGQDTNETIFSYLYSLISIIFPEDIKGQGTSAQLLEYRTQLASLSKIKSLRKKIKRRL

SEQ ID NO: 176 - protein, 303 aa, Babesia microti
MQHHHHHHHFITFFLTSGNVFAGNGDVNQYSSDFGRALNDLMIAFNEAKKMYAKFSEQITDTMIHTCKNSIDILEADEKNGGHKNYLEKKEIELKSKIVEFNAIFSNIDLNNSTVKNEIIKLLNDISTISTDIKSIVDEIYYKALGTIEGENAENFEYEIKKKKAELLRNLLNDNIKPIMGYLTEIYNMHIPIISNKSEFNDIKKAFEKHELEANVLISKILENNQNFGTNFNDILNEVNGAIEEFNKTIDVMNNTIGDLGIVIDSGIISSIKSYISTIAKISNSIIPGQMALVFTALILILN

SEQ ID NO: 177 - protein, 230 aa, Babesia microti
MQHHHHHHRLTLTLATNTRGGAGTDATSVSIANSIPTSAATAAQSTTAATSTTAATSTTSATSTTSATSTTATTSTTTATSTTTATSTTATTSTTAATSTISPSLETTQDVAVTNIVNLNINEIGFVDQVPEGLSSSYVFSTDGIFTKVTPATGFSIGCVIFGNQLIPQSMDVITRTVSYTTKYPLIVVRIQDKTSSSTSTVYYEQSGLQSSKFVLRDDPEFTSQLTSSF

SEQ ID NO: 178 - protein, 185 aa, Babesia microti
MQHHHHHHEGYLDLDLNSKIGNFISAIELTNLTNTVKSASVHPPQLKVLALKFGNKIVDVEETGRTFVTFDEKLNSIEIITFENDGTMTSKFYSRESLDSTTYIGHASTYTLPEVLTRSLCGKEDLCTLDITDLLLKEISAKKLEECRKKNASSGTPSGGTPSNVPEECVIRTNLQMVMKKNARA

SEQ ID NO: 179 - protein, 260 aa, Babesia microti
MQHHHHHHGSRFSEMGSRFSVSPWAWLECPSCLPSPLFQVTMSPSQSPRWSSCPPLSSWLLPHPRHIPIKDCRLSYCYPCRVLMPLRPGTSSASVPSRPHSAPPHVAGPPSAPRDLQYSLSRSPLALRLRWLPPADSGGRSDVTYSLLCLLCGRDGPAGACQPCGPRVAFVPRQAGLRERAATLLHLRPGARYTVRVAALNGVSGPAAAAEATYAQVTVSTGPGGEATRPSGVRPPPQPQFPLCIPSHSGTHVTTPHAPG

SEQ ID NO: 180 - protein, 297 aa, Babesia microti
MQHHHHHHEANIRTNQTVRIYLALQESYLHTHAHVLSVCTATSTTSATSTTATTSTTTATSTTTATSTTATTSTTAATSTISPSLETTQDVAVTNIVNLNINEIGFVDQVPEGLSSSYVFSTDGIFTKVTPATGFSIGCVIFGNQLIPQSMDVITRTVSYTTKYPLIVVRIQDKTSSSTSTVYYEQSGLQSSKFVLRDDPEFIIPQNRSSTYTVNDITYKSFDISSADDNEFLKISLSDGSMLYTNNPDSKIYISEVKVGEITIPINITSQYTLIKLSFNGELVELYTTGCFGEHNI

SEQ ID NO: 181 - protein, 25 aa, B. microti
TGTAGTTTSSEGAGSDKAGTGTSGT

SEQ ID NO: 182 - protein, 25 aa, B. microti
EAGGTSGTTTSSGAASGKAGTGTAG

SEQ ID NO: 183 - protein, 25 aa, B. microti
TGNGGTESGGTAGTTTSSGTEAGGT

SEQ ID NO: 184 - protein, 25 aa, B. microti
TESGGAGSGTGTSVSATSTLTGNGG

SEQ ID NO: 185 - protein, 25 aa, B. microti
YIVGAGVEAVTVSVSATSNGTESGG

SEQ ID NO: 186 - protein, 25 aa, B. microti
GIKINRDVISSYKLLLSTITYIVGA

SEQ ID NO: 187 - protein, 26 aa, B. microti
TCANTKFEALNDLIISDCEKKGIKIN

SEQ ID NO: 188 - protein, 25 aa, B. microti
ILDNDEDYKINFREMVNEVTCANTK

SEQ ID NO: 189 - protein, 27 aa, B. microti
PSGHASNAKIPGIMTLTLFALLTFIVN

SEQ ID NO: 190 - protein, 25 aa, B. microti
GTSGTTTSSGTGAGGAGSGGPSGHA

SEQ ID NO: 191 - protein, 25 aa, B. microti
DDIKKAFDECKSNAIILKKKILDND

SEQ ID NO: 192 - protein, 25 aa, B. microti
GNAGIKSYDTQTTQEICEECEEGHD

SEQ ID NO: 193 - protein, 25 aa, B. microti
TQEICEECEEGHDKINKNKSGNAGI

SEQ ID NO: 194 - protein, 50 aa, B. microti
GKPNTNKSEKAERKSHDTQTTQEICEEGGTSGTTTSSGAASGKAGTGTAG

SEQ ID NO: 195 - protein, 26 aa, B. microti
GKPNTNKSEKAERKSHDTQTTQEICE

SEQ ID NO: 196 - protein, 25 aa, B. microti
LDNLLRLTAQEIYEERKEGHGKPNT

SEQ ID NO: 197 - protein, 25 aa, B. microti
SEKTERKSHDTQTPQEIYEELDNLL

SEQ ID NO: 198 - protein, 25 aa, B. microti
IKSYDTQTPQETSDAHEEEHGNLNK

SEQ ID NO: 199 - protein, 26 aa, B. microti
ICEECEEGHDKINKNKSGNAGIKSYD

SEQ ID NO: 200 - protein, 25 aa, B. microti
TAQETSDDHEEGNGKLNTNKSEKTE

SEQ ID NO: 201 - protein, 25 aa, B. microti
TNKSEKAERKSHDTQTTQEICEECE

SEQ ID NO: 202 - protein, 25 aa, B. microti
EEGHDKINKNKSGNAGIKSYDTQTP

SEQ ID NO: 203 - protein, 25 aa, B. microti
DTQTPQETSDAHEEGHDKINTNKSE

SEQ ID NO: 204 - DNA, 1359 bp, Babesia microti
taaaatatgacaaaagatttaatgaacatactgacatgaatggtattcattattattatattgatggtagtttacttgcgagtggcgaagttacatctaattttcgttatatttctaaagaatatgaatatgagcatacagaattagcaaaagagcattgcaagaaagaaaaatgtgtaaatgtggataacattgaggataataatttgaaaatatatgcgaaacagtttaaatctgtagttactactccagctgatgtagcgggtgtgtcagatggattttttatacgtggccaaaatcttggtgctgtgggcagtgtaaatgaacaacctaatactgttggtatgagtttagaacaattcatcaagaacgagctttattcttttagtaatgaaatttatcatacaatatctagtcaaatcagtaattctttcttaataatgatgtctgatgcaattgttaaacatgataactatattttaaaaaaagaaggtgaaggctgtgaacaaatctacaattatgaggaatttatagaaaagttgaggggtgctagaagtgaggggaataatatgtttcaggaagctctgataaggtttaggaatgctagtagtgaagaaatggttaatgctgcaagttatctatccgccgcccttttcagatataaggaatttgatgatgaattattcaaaaaggccaacgataattttggacgcgatgatggatatgattttgattatataaatacaaagaaagagttagttatacttgccagtgtgttggatggtttggatttaataatggaacgtttgatcgaaaatttcagtgatgtcaataatacagatgatattaagaaggcatttgacgaatgcaaatctaatgctattatattgaagaaaaagatacttgacaatgatgaagattataagattaattttagggaaatggtgaatgaagtaacatgtgcaaacacaaaatttgaagccctaaatgatttgataatttccgactgtgagaaaaaaggtattaagataaacagagatgtgatttcaagctacaaattgcttctttccacaatcacctatattgttggagctggagttgaagctgtaactgttagtgtgtctgctacatctaatggaactggtggtggtggagctgctagtggaactggaactagtggaactactacgtctagtgaaggtgctggtagtggtaaagctggaactggaactagtggaactactacgtctagtggaactggtgctggtggagctggtagtggtggacctagtggacatgcttctaatgcaaaaattcctggaataatgacactaactctatttgcattattaacattta

SEQ ID NO: 205 - DNA, 25 bp, Babesia microti
aaatgttaataatgcaaatagagtt

SEQ ID NO: 206 - DNA, 26 bp, Babesia microti
caatgaataatgatacaaataaatgg

SEQ ID NO: 207 - protein, 54 aa, Babesia microti
YIVGAGVEAVTVSVSATSNGTGGGGAASGTGTSGTTTSSEGAGSGKAGTGTSGT

SEQ ID NO: 208 - protein, 45 aa, Babesia microti
YIVGAGVEAVTVSVSATSNGTESGGAGSGTGTSVSATSTLTGNGG

SEQ ID NO: 209 - protein, 452 aa, Babesia microti
KYDKRFNEHTDMNGIHYYYIDGSLLASGEVTSNFRYISKEYEYEHTELAKEHCKKEKCVNVDNIEDNNLKIYAKQFKSVVTTPADVAGVSDGFFIRGQNLGAVGSVNEQPNTVGMSLEQFIKNELYSFSNEIYHTISSQISNSFLIMMSDAIVKHDNYILKKEGEGCEQIYNYEEFIEKLRGARSEGNNMFQEALIRFRNASSEEMVNAASYLSAALFRYKEFDDELFKKANDNFGRDDGYDFDYINTKKELVILASVLDGLDLIMERLIENFSDVNNTDDIKKAFDECKSNAIILKKKILDNDEDYKINFREMVNEVTCANTKFEALNDLIISDCEKKGIKINRDVISSYKLLLSTITYIVGAGVEAVTVSVSATSNGTGGGGAASGTGTSGTTTSSEGAGSGKAGTGTSGTTTSSGTGAGGAGSGGPSGHASNAKIPGIMTLTLFALLTF

SEQ ID NO: 210 - DNA, 2079 bp, Babesia microti
aatccaacatctagcctagttagtatatataggttaatatcacattatagattatctttggatgattggttattatataacatgtcgctgaatgacgattattttgctagataatataactaccggtgattctgaggacctactttaaagagaataattaacatatctaccagaatcagttccaatttatgtattttaaagctaatcactactcgaaaactacggtgaaaatggaaaaacaagtggaagctgtatgtcgtggaaagtcactacattttatgtgggcaaatttaataattctaaatactatgtttttgatgttaaaaagcgaaaaacacactttaatgcacattttaacatcatctgtataatatatatatcagcgttgaaatcatatggcaaaggtaataaagcgttacattttgagcgaataaaggcacatatgcaaacgtatgaagccttgtatatttgtggaattatattatgctagtaatttgtgattaataatggcaatatttatatacaaatattcgagcgttctattatatgcatgcacataattaatcacaaactctcatatcatggggcggtttcgcccatcataaacattactgttagcactctggtagattagcatggtgaatctctcgatacctgggctactgttgctttccgcatattccttaaattctgcaagtgcgggggatgtatatgagatatcttctggtaatccacccgacatagagccaacatctacttctctagaaacaaatgtagttaccaactatattccagaacccaatgcggattcagaatctgtacatgttgaaatccaggaacatgataacatcaatccacaagacgcttgcgatagtgagccgctcgaacaaatggattctgataccagggtgttgcccgaaagtttggatgagggggtaccacaccaattctctagattagggcaccactcagacatggcatctgatataaatgatgaagaaccatcatttaaaatcggcgagaatgacataattcaaccaccctgggaagatacagctccataccattcaatagatgatgaagagcttgacaacttaatgagactaacggcgcaagaaacaagtgacgatcatgaagaagggaatggcaaactcaatacgaataaaagtgagaagactgaaagaaaatcgcatgatactcagacaccgcaagaaatatatgaagagcttgacaacttactgagactaacggcacaagaaatatatgaagagcgtaaagaagggcatggcaaacccaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagggcatgacaaaatcaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagagcatggcaatctcaataagaataaaagtgggaaggctggaataaaatcgcataatactcagacaccgctgaaaaaaaaagacttttgtaaagaagggtgtcatggttgcaataataagcccgaggataatgaaagagacccgtcgtcgcctgatgatgatggtggctgcgaatgcggcatgacgaatcactttgtctttgactacaagacaacactcttgttaaagagcctcaagactgaaacatccactcattattacattgccatggctgcaatttttactatttcattattcccatgcatgtttaaggctttcc

SEQ ID NO: 211 - protein, 481 aa, Babesia microti
MVNLSIPGLLLLSAYSLNSASAGDVYEISSGNPPDIEPTSTSLETNVVTNYIPEPNADSESVHVEIQEHDNINPQDACDSEPLEQMDSDTRVLPESLDEGVPHQFSRLGHHSDMASDINDEEPSFKIGENDIIQPPWEDTAPYHSIDDEELDNLMRLTAQETSDDHEEGNGKLNTNKSEKTERKSHDTQTPQEIYEELDNLLRLTAQEIYEERKEGHGKPNTNKSEKAERKSHDTQTTQEICEECEEGHDKINKNKSGNAGIKSYDTQTTQEICEECEEGHDKINKNKSGNAGIKSYDTQTPQETSDAHEEGHDKINTNKSEKAERKSHDTQTTQEICEECEEGHDKINKNKSGNAGIKSYDTQTPQETSDAHEEEHGNLNKNKSGKAGIKSHNTQTPLKKKDFCKEGCHGCNNKPEDNERDPSSPDDDGGCECGMTNHFVFDYKTTLLLKSLKTETSTHYYIAMAAIFTISLFPCMFKAF

SEQ ID NO: 212 - protein, 20 aa, Babesia microti
NSASAGDVYEISSGNPPDIE

SEQ ID NO: 213 - protein, 20 aa, Babesia microti
PPDIEPTSTSLETNVVTNYI

SEQ ID NO: 214 - protein, 20 aa, Babesia microti
VTNYIPEPNADSESVHVEIQ

SEQ ID NO: 215 - protein, 20 aa, Babesia microti
HVEIQEHDNINPQDACDSEP

SEQ ID NO: 216 - protein, 21 aa, Babesia microti
ACDSEPLEQMDSDTRVLPESL

SEQ ID NO: 217 - protein, 20 aa, Babesia microti
LPESLDEGVPHQFSRLGHHS

SEQ ID NO: 218 - protein, 20 aa, Babesia microti
LGHHSDMASDINDEEPSFKI

SEQ ID NO: 219 - protein, 20 aa, Babesia microti
PSFKIGENDIIQPPWEDTAP

SEQ ID NO: 220 - protein, 20 aa, Babesia microti
EDTAPYHSIDDEELDNLMRL

SEQ ID NO: 221 - protein, 20 aa, Babesia microti
HSIDDEELDNLMRLTAQETS

SEQ ID NO: 222 - protein, 20 aa, Babesia microti
TTLLLKSLKTETSTHYYIAM

SEQ ID NO: 223 - protein, 21 aa, Babesia microti
ECGMTNHFVFDYKTTLLLKSL

SEQ ID NO: 224 - protein, 20 aa, Babesia microti
DNERDPSSPDDDGGCECGMT

(19) United States
(12) Patent Application Publication
(10) Pub. No.: US 2003/0109689 A1
(43) Pub. Date: Jun. 12, 2003

(54) Compounds and methods for the diagnosis and treatment of B. microti infection

(76) Inventors: Steven G. Reed, Bellevue, WA (US); Michael J. Lodes, Seattle, WA (US); Raymond L. Houghton, Bothell, WA (US); Paul R. Sleath, Seattle, WA (US); Patricia D. McNeill, Federal Way, WA (US); Mary J. Homer, Seattle, WA (US); Heather Secrist, Seattle, WA (US)

Correspondence Address: CORIXA CORPORATION, 1124 COLUMBIA STREET, SUITE 200, SEATTLE, WA, 98104, US

(21) Appl. No.: 09/853,079

(22) Filed: May 9, 2001

Related U.S. Application Data
(63) Continuation-in-part of application No. 09/794,764, filed on Feb. 26, 2001.
(63) Continuation-in-part of application No. 09/737,178, filed on Dec. 13, 2000, now abandoned.
(63) Continuation-in-part of application No. 09/685,436, filed on Oct. 10, 2000.
(63) Continuation-in-part of application No. 09/656,688, filed on Sep. 7, 2000, now abandoned.
(63) Continuation-in-part of application No. 09/605,724, filed on Jun. 27, 2000, now abandoned.
(63) Continuation-in-part of application No. 09/569,098, filed on May 10, 2000.
(63) Continuation-in-part of application No. 09/528,784, filed on Mar. 17, 2000, now Pat. No. 6,451,315.
(63) Continuation-in-part of application No. 09/286,488, filed on Apr. 5, 1999.
(63) Continuation-in-part of application No. 08/990,571, filed on Dec. 11, 1997, now Pat. No. 6,214,971.
(63) Continuation-in-part of application No. 08/845,258, filed on Apr. 24, 1997, now Pat. No. 6,183,976.
(63) Continuation-in-part of application No. 08/723,142, filed on Oct. 1, 1996, now Pat. No. 6,306,396.

(51) Int. Cl. C12Q 1/68; C07H 21/02; C07H 21/04

(52) U.S. Cl. 536/23.1; 435/6

(57) ABSTRACT

Compounds and methods for the diagnosis and treatment of B. microti infection are disclosed. The compounds provided include polypeptides that contain at least one antigenic portion of a B. microti antigen and DNA sequences encoding such polypeptides. Antigenic epitopes of such antigens are also provided, together with pharmaceutical compositions and immunogenic compositions comprising such polypeptides, DNA sequences or antigenic epitopes. Diagnostic kits containing such polypeptides, DNA sequences or antigenic epitopes and a suitable detection reagent may be used for the detection of B. microti infection in patients and biological samples. Antibodies directed against such polypeptides and antigenic epitopes are also provided.

### CROSS-REFERENCE TO RELATED APPLICATIONS

This application is related to U.S. patent application Ser. No. 09/794,764, filed Feb. 26, 2001 (pending); U.S. patent application Ser. No. 09/737,178, filed Dec. 13, 2000 (pending); U.S. patent application Ser. No. 09/685,436, filed Oct. 10, 2000 (pending); U.S. patent application Ser. No. 09/656,688, filed Sep. 7, 2000 (pending); U.S. application Ser. No. 09/605,724, filed Jun. 27, 2000 (pending); U.S. application Ser. No. 09/569,098, filed May 10, 2000 (pending); U.S. application Ser. No. 09/528,784, filed Mar. 17, 2000 (pending); U.S. application Ser. No. 09/286,488, filed Apr. 5, 1999 (pending); U.S. application Ser. No. 08/990,571, filed Dec. 11, 1997 (allowed); U.S. application Ser. No. 08/845,258, filed Apr. 24, 1997 (allowed); U.S. application Ser. No. 08/723,142, filed Oct. 1, 1996 (pending); each a continuation-in-part of the previous application and all incorporated herein by reference.

### TECHNICAL FIELD

The present invention relates generally to the detection of Babesia microti infection. In particular, the invention is related to polypeptides comprising a B. microti antigen, to antigenic epitopes of such an antigen and the use of such polypeptides and antigenic epitopes for the serodiagnosis and treatment of B. microti infection.

### BACKGROUND OF THE INVENTION

Babesiosis is a malaria-like illness caused by the rodent parasite Babesia microti (B. microti) which is generally transmitted to humans by the same tick that is responsible for the transmission of Lyme disease and ehrlichiosis, thereby leading to the possibility of co-infection with babesiosis, Lyme disease and ehrlichiosis from a single tick bite. While the number of reported cases of B. microti infection in the United States is increasing rapidly, infection with B. microti, including co-infection with Lyme disease, often remains undetected for extended periods of time. Babesiosis is potentially fatal, particularly in the elderly and in patients with suppressed immune systems. Patients infected with both Lyme disease and babesiosis have more severe symptoms and prolonged illness compared to those with either infection alone.

The preferred treatments for Lyme disease, ehrlichiosis and babesiosis are different, with penicillins, such as doxycycline and amoxicillin, being most effective in treating Lyme disease, tetracycline being preferred for the treatment of ehrlichiosis, and anti-malarial drugs, such as quinine and clindamycin, being most effective in the treatment of babesiosis. Accurate and early diagnosis of B. microti infection is thus critical but methods currently employed for diagnosis are problematic.

All three tick-borne illnesses share the same flu-like symptoms of muscle aches, fever, headaches and fatigue, thus making clinical diagnosis difficult. Microscopic analysis of blood samples may provide false-negative results when patients are first seen in the clinic. Indirect fluorescent antibody staining methods for total immunoglobulins to B. microti may be used to diagnose babesiosis infection, but such methods are time-consuming and expensive. There thus remains a need in the art for improved methods for the detection of B. microti infection.

### SUMMARY OF THE INVENTION

The present invention provides compositions and methods for the diagnosis and treatment of B. microti infection. In one aspect, polypeptides are provided comprising an immunogenic portion of a B. microti antigen, or a variant of such an antigen that differs only in conservative substitutions and/or modifications. In one embodiment, the antigen comprises an amino acid sequence encoded by a DNA sequence selected from the group consisting of (a) sequences recited in SEQ ID NOs:1-17, 37, 40, 42, 45, 50, 51, 91-119, 128-131, 135, 204 and 210; (b) the complements of said sequences; and (c) sequences that hybridize to a sequence of (a) or (b) under moderately stringent conditions. In certain embodiments, such polypeptides comprise a sequence selected from the group consisting of SEQ ID NO: 181-203, 207-209 and 211-224.

In another aspect, the present invention provides an antigenic epitope of a B. microti antigen comprising the amino acid sequence -X₁-X₂-X₃-X₄-X₅-Ser- (SEQ ID NO:35), wherein X₁ is Glu or Gly, X₂ is Ala or Thr, X₃ is Gly or Val, X₄ is Trp or Gly and X₅ is Pro or Ser. In one embodiment of this aspect, X₁ is Glu, X₂ is Ala and X₃ is Gly. In a second embodiment X₁ is Gly, X₂ is Thr and X₅ is Pro. The present invention further provides polypeptides comprising at least two of the above antigenic epitopes, the epitopes being contiguous.

In yet another aspect, the present invention provides an antigenic epitope of a B. microti antigen comprising an amino acid sequence selected from the group consisting of SEQ ID NOs:36 and 39, together with polypeptides comprising at least two such antigenic epitopes, the epitopes being contiguous.

In a related aspect, polynucleotides encoding the above polypeptides, recombinant expression vectors comprising these polynucleotides and host cells transformed or transfected with such expression vectors are also provided.

In another aspect, the present invention provides fusion proteins comprising either a first and a second inventive polypeptide, a first and a second inventive antigenic epitope, or, alternatively, an inventive polypeptide and an inventive antigenic epitope. In specific embodiments, fusion proteins comprising an amino acid sequence of SEQ ID NO:85, 87, 144 or 211 are provided.

In further aspects of the subject invention, methods and diagnostic kits are provided for detecting B. microti infection in a patient. In one embodiment, the method comprises: (a) contacting a biological sample with at least one polypeptide comprising an immunogenic portion of a B. microti antigen; and (b) detecting in the sample the presence of antibodies that bind to the polypeptide, thereby detecting B. microti infection in the biological sample. In other embodiments, the methods comprise: (a) contacting a biological sample with at least one of the above polypeptides or antigenic epitopes; and (b) detecting in the sample the presence of antibodies that bind to the polypeptide or antigenic epitope. Suitable biological samples include whole blood, sputum, serum, plasma, saliva, cerebrospinal fluid and urine. The diagnostic kits comprise one or more of the above polypeptides or antigenic epitopes in combination with a detection reagent.

The present invention also provides methods for detecting B. microti infection comprising: (a) obtaining a biological sample from a patient; (b) contacting the sample with at least two oligonucleotide primers in a polymerase chain reaction, at least one of the oligonucleotide primers being specific for a DNA sequence encoding the above polypeptides; and (c) detecting in the sample a DNA sequence that amplifies in the presence of the first and second oligonucleotide primers. In one embodiment, the oligonucleotide primer comprises at least about 10 contiguous nucleotides of a DNA sequence encoding the above polypeptides.

In a further aspect, the present invention provides a method for detecting B. microti infection in a patient comprising: (a) obtaining a biological sample from the patient; (b) contacting the sample with an oligonucleotide probe specific for a DNA sequence encoding the above polypeptides; and (c) detecting in the sample a DNA sequence that hybridizes to the oligonucleotide probe. In one embodiment of this aspect, the oligonucleotide probe comprises at least about 15 contiguous nucleotides of a DNA sequence encoding the above polypeptides.

In yet another aspect, the present invention provides antibodies, both polyclonal and monoclonal, that bind to the polypeptides described above, as well as methods for their use in the detection of B. microti infection.

Within other aspects, the present invention provides pharmaceutical compositions that comprise one or more of the above polypeptides or antigenic epitopes, or a polynucleotide encoding such polypeptides, and a physiologically acceptable carrier. The invention also provides immunogenic compositions comprising one or more of the inventive polypeptides or antigenic epitopes and an immunostimulant, together with immunogenic compositions comprising one or more polynucleotides encoding such polypeptides and an immunostimulant.

In yet another aspect, methods are provided for inducing protective immunity in a patient, comprising administering to a patient an effective amount of one or more of the above pharmaceutical compositions or immunogenic compositions.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 shows the genomic sequence of the B. microti antigen BMNI-3 (SEQ ID NO: 3) including a translation of the putative open reading frame (SEQ ID NO: 49). An internal six amino acid repeat sequence (SEQ ID NO:35) is indicated by vertical lines within the open reading frame.

FIG. 2a shows the reactivity of the B. microti antigens BMNI-3 and BMNI-6, and the peptides BABS-1 and BABS-4 with sera from B. microti-infected individuals and from normal donors as determined by ELISA. FIG. 2b shows the reactivity of the B. microti antigens BMNI-4 and BMNI-15 with sera from B. microti-infected individuals and from normal donors as determined by ELISA.

FIG. 3 shows the reactivity of the B. microti antigens MN-10 and BMNI-20 with sera from B. microti-infected patients and from normal donors as determined by ELISA.

FIG. 4 shows the results of Western blot analysis of representative B. microti antigens of the present invention.

FIG. 5 shows the reactivity of purified recombinant B. microti antigen BMNI-3 with sera from B. microti-infected patients, Lyme disease-infected patients, ehrlichiosis-infected patients and normal donors as determined by Western blot analysis.

FIG. 6 shows an alignment of the repeat region of different homologues of the B. microti antigen BMNI-6, illustrating the geographic variation in the number and location of the repeats.

### DETAILED DESCRIPTION OF THE INVENTION

As noted above, the present invention is generally directed to compositions and methods for the diagnosis and treatment of B. microti infection. In one aspect, the compositions of the subject invention include polypeptides that comprise at least one immunogenic portion of a B. microti antigen, or a variant thereof.

As used herein, the term “polypeptide” encompasses amino acid chains of any length, including full length proteins (i.e., antigens), wherein the amino acid residues are linked by covalent peptide bonds. Thus, a polypeptide comprising an immunogenic portion of one of the above antigens may consist entirely of the immunogenic portion, or may contain additional sequences. The additional sequences may be derived from the native B. microti antigen or may be heterologous, and such sequences may (but need not) be immunogenic.

An “immunogenic portion” of an antigen is a portion that is capable of reacting with sera obtained from a B. microti-infected individual (i.e., generates an absorbance reading with sera from infected individuals that is at least three standard deviations above the absorbance obtained with sera from uninfected individuals, in a representative ELISA assay described herein). Polypeptides comprising at least an immunogenic portion of one or more B. microti antigens as described herein may generally be used, alone or in combination, to detect B. microti in a patient.

Polynucleotides encoding the inventive polypeptides are also provided. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

The compositions and methods of the present invention also encompass variants of the above polypeptides and polynucleotides. Such variants include, but are not limited to, naturally occurring allelic variants of the inventive sequences.

Polynucleotides may comprise a native sequence (i.e., an endogenous sequence that encodes a protein or a portion thereof) or may comprise a variant, or a biological or antigenic functional equivalent of such a sequence. Polynucleotide variants may contain one or more substitutions, additions, deletions and/or insertions, as further described below, preferably such that the immunogenicity of the encoded polypeptide is not diminished, relative to a native tumor protein. The effect on the immunogenicity of the encoded polypeptide may generally be assessed as described herein. The term “cvariants” also encompasses homologous genes of xenogenic origin.

When comparing polynucleotide or polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Therefore, the present invention encompasses polynucleotide and polypeptide sequences having substantial identity to the sequences disclosed herein, for example those comprising at least 50% sequence identity, preferably at least 55%, 60%, 65%, 70%, 75%, 80%, 85%, 90%, 95%, 96%, 97%, 98%, or 99% or higher, sequence identity compared to a polynucleotide or polypeptide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

In additional embodiments, the present invention provides isolated polynucleotides and polypeptides comprising various lengths of contiguous stretches of sequence identical to or complementary to one or more of the sequences disclosed herein. For example, polynucleotides are provided by this invention that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative DNA segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

In other embodiments, the present invention is directed to polynucleotides that are capable of hybridizing under moderately stringent conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

A polypeptide “variant,” as used herein, is a polypeptide that differs from a native protein in one or more substitutions, deletions, additions and/or insertions, such that the immunogenicity of the polypeptide is not substantially diminished. In other words, the ability of a variant to react with antigen-specific antisera may be enhanced or unchanged, relative to the native protein, or may be diminished by less than 50%, and preferably less than 20%, relative to the native protein. Such variants may generally be identified by modifying one of the above polypeptide sequences and evaluating the reactivity of the modified polypeptide with antigen-specific antibodies or antisera as described herein. Preferred variants include those in which one or more portions, such as an N-terminal leader sequence or transmembrane domain, have been removed. Other preferred variants include variants in which a small portion (e.g., 1-30 amino acids, preferably 5-15 amino acids) has been removed from the N- and/or C-terminal of the mature protein.

Polypeptide variants encompassed by the present invention include those exhibiting at least about 70%, 75%, 80%, 85%, 90%, 91%, 92%, 93%, 94%, 95%, 96%, 97%, 98%, or 99% or more identity (determined as described above) to the polypeptides disclosed herein.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

In general, B. microti antigens, and polynucleotides encoding such antigens, may be prepared using any of a variety of procedures. For example, polynucleotides encoding B. microti antigens may be isolated from a B. microti genomic or cDNA expression library by screening with sera from B. microti-infected individuals as described below in Example 1, and sequenced using techniques well known to those of skill in the art. Polynucleotides encoding B. microti antigens may also be isolated by screening an appropriate B. microti expression library with anti-sera (e.g., rabbit) raised specifically against B. microti antigens.

Antigens may be induced from such clones and evaluated for a desired property, such as the ability to react with sera obtained from a B. microti-infected individual as described herein. Alternatively, antigens may be produced recombinantly, as described below, by inserting a polynucleotide that encodes the antigen into an expression vector and expressing the antigen in an appropriate host. Antigens may be partially sequenced using, for example, traditional Edman chemistry. See Edman and Berg, Eur. J. Biochem. 80:116-132,1967.

Polynucleotides encoding antigens may also be obtained by screening an appropriate B. microti cDNA or genomic DNA library for polynucleotides that hybridize to degenerate oligonucleotides derived from partial amino acid sequences of isolated antigens. Degenerate oligonucleotides for use in such a screen may be designed and synthesized, and the screen may be performed, as described (for example) in Sambrook et al., Molecular Cloning: A Laboratory Manual, Cold Spring Harbor Laboratories, Cold Spring Harbor, N.Y. (and references cited therein). Polymerase chain reaction (PCR) may also be employed, using the above oligonucleotides in methods well known in the art, to isolate a nucleic acid probe from a cDNA or genomic library. The library screen may then be performed using the isolated probe.

Synthetic polypeptides having fewer than about 100 amino acids, and generally fewer than about 50 amino acids, may be generated using techniques well known in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division, Foster City, Calif., and may be operated according to the manufacturer's instructions.

Immunogenic portions of B. microti antigens may be prepared and identified using well known techniques, such as those summarized in Paul, Fundamental Immunology, 3d ed., Raven Press, 1993, pp. 243-247 and references cited therein. Such techniques include screening polypeptide portions of the native antigen for immunogenic properties. The representative ELISAs described herein may generally be employed in these screens. An immunogenic portion of a polypeptide is a portion that, within such representative assays, generates a signal in such assays that is substantially similar to that generated by the full length antigen. In other words, an immunogenic portion of a B. microti antigen generates at least about 20%, and preferably about 100%, of the signal induced by the full length antigen in a model ELISA as described herein.

Portions and other variants of B. microti antigens may be generated by synthetic or recombinant means. Variants of a native antigen may generally be prepared using standard mutagenesis techniques, such as oligonucleotide-directed site-specific mutagenesis. Sections of the DNA sequence may also be removed using standard techniques to permit preparation of truncated polypeptides.

Recombinant polypeptides containing portions and/or variants of a native antigen may be readily prepared from a polynucleotide encoding the polypeptide using a variety of techniques well known to those of ordinary skill in the art. For example, supernatants from suitable host/vector systems which secrete recombinant protein into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant protein.

Any of a variety of expression vectors known to those of ordinary skill in the art may be employed to express recombinant polypeptides as described herein. Expression may be achieved in any appropriate host cell that has been transformed or transfected with an expression vector containing a polynucleotide that encodes a recombinant polypeptide. Suitable host cells include prokaryotes, yeast and higher eukaryotic cells. Preferably, the host cells employed are E. coli, yeast or a mammalian cell line, such as COS or CHO. The polynucleotides expressed in this manner may encode naturally occurring antigens, portions of naturally occurring antigens, or other variants thereof.

In another aspect, the present invention provides epitope repeat sequences, or antigenic epitopes, of a B. microti antigen, together with polypeptides comprising at least two such contiguous antigenic epitopes. As used herein an “epitope” is a portion of an antigen that reacts with sera from B. microti-infected individuals (i.e. an epitope is specifically bound by one or more antibodies present in such sera). As discussed above, epitopes of the antigens described in the present application may be generally identified using techniques well known to those of skill in the art.

In one embodiment, antigenic epitopes of the present invention comprise the amino acid sequence -X₁-X₂-X₃-X₄-X₅-Ser- (SEQ ID NO:35), wherein X₁ is Glu or Gly, X₂ is Ala or Thr, X₃ is Gly or Val, X₄ is Trp or Gly, and X₅ is Pro or Ser. In another embodiment, the antigenic epitopes of the present invention comprise an amino acid sequence selected from the group consisting of SEQ ID NO: 36 and 39. As discussed in more detail below, antigenic epitopes provided herein may be employed in the diagnosis and treatment of B. microti infection, either alone or in combination with other B. microti antigens or antigenic epitopes. Antigenic epitopes and polypeptides comprising such epitopes may be prepared by synthetic means, as described generally above and in detail in Example 2.

In general, regardless of the method of preparation, the polypeptides, polynucleotides and antigenic epitopes disclosed herein are prepared in an isolated, 10 substantially pure, form. Preferably, the polypeptides and antigenic epitopes are at least about 80% pure, more preferably at least about 90% pure and most preferably at least about 99% pure.

In a further aspect, the present invention provides fusion proteins comprising either a first and a second inventive polypeptide, a first and a second inventive antigenic epitope or an inventive polypeptide and an antigenic epitope of the present invention, together with variants of such fusion proteins. The fusion proteins of the present invention may also include a linker peptide between the polypeptides or antigenic epitopes.

A polynucleotide encoding a fusion protein of the present invention is constructed using known recombinant DNA techniques to assemble separate polynucleotides encoding, for example, the first and second polypeptides into an appropriate expression vector. The 3′ end of a polynucleotide encoding the first polypeptide is ligated, with or without a peptide linker, to the 5′ end of a polynucleotide encoding the second polypeptide so that the reading frames of the sequences are in phase to permit mRNA translation of the two polynucleotides into a single fusion protein that retains the biological activity of both the first and the second polypeptides.

A peptide linker sequence may be employed to separate the first and the second polypeptides by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8562, 1986; U.S. Pat. No. 4,935,233 and U.S. Pat. No. 4,751,180. The linker sequence may be from 1 to about 50 amino acids in length. Peptide linker sequences are not required when the first and second polypeptides have non-essential N-terminal amino acid regions that can be used to separate the functional domains and prevent steric hindrance.

In another aspect, the present invention provides methods for using polypeptides comprising an immunogenic portion of a B. microti antigen and/or the antigenic epitopes described above to diagnose babesiosis. In this aspect, methods are provided for detecting B. microti infection in a biological sample, using one or more of the above polypeptides and antigenic epitopes, alone or in combination. For clarity, the term “polypeptide” will be used when describing specific embodiments of the inventive diagnostic methods. However, it will be clear to one of skill in the art that the antigenic epitopes of the present invention may also be employed in such methods.

As used herein, a “biological sample” is any antibody-containing sample obtained from a patient. Preferably, the sample is whole blood, sputum, serum, plasma, saliva, cerebrospinal fluid or urine. More preferably, the sample is a blood, serum or plasma sample obtained from a patient. The polypeptides are used in an assay, as described below, to determine the presence or absence of antibodies to the polypeptide(s) in the sample, relative to a predetermined cut-off value. The presence of such antibodies indicates previous sensitization to B. microti antigens which may be indicative of babesiosis.

In embodiments in which more than one polypeptide is employed, the polypeptides used are preferably complementary (i.e., one component polypeptide will tend to detect infection in samples where the infection would not be detected by another component polypeptide). Complementary polypeptides may generally be identified by using each polypeptide individually to evaluate serum samples obtained from a series of patients known to be infected with B. microti. After determining which samples test positive (as described below) with each polypeptide, combinations of two or more polypeptides may be formulated that are capable of detecting infection in most, or all, of the samples tested.

A variety of assay formats are known to those of ordinary skill in the art for using one or more polypeptides to detect antibodies in a sample. See, e.g., Harlow and Lane, Antibodies: A Laboratory Manual, Cold Spring Harbor Laboratory, 1988, which is incorporated herein by reference. In a preferred embodiment, the assay involves the use of polypeptide immobilized on a solid support to bind to and remove the antibody from the sample. The bound antibody may then be detected using a detection reagent that contains a reporter group. Suitable detection reagents include antibodies that bind to the antibody/polypeptide complex and free polypeptide labeled with a reporter group (e.g., in a semi-competitive assay). Alternatively, a competitive assay may be utilized, in which an antibody that binds to the polypeptide is labeled with a reporter group and allowed to bind to the immobilized antigen after incubation of the antigen with the sample. The extent to which components of the sample inhibit the binding of the labeled antibody to the polypeptide is indicative of the reactivity of the sample with the immobilized polypeptide.

The solid support may be any solid material known to those of ordinary skill in the art to which the antigen may be attached. For example, the solid support may be a test well in a microtiter plate, or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

The polypeptides may be bound to the solid support using a variety of techniques known to those of ordinary skill in the art. In the context of the present invention, the term “bound” refers to both noncovalent association, such as adsorption, and covalent attachment (which may be a direct linkage between the antigen and functional groups on the support or may be a linkage by way of a cross-linking agent). Binding by adsorption to a well in a microtiter plate or to a membrane is preferred. In such cases, adsorption may be achieved by contacting the polypeptide, in a suitable buffer, with the solid support for a suitable amount of time. The contact time varies with temperature, but is typically between about 1 hour and 1 day. In general, contacting a well of a plastic microtiter plate (such as polystyrene or polyvinylchloride) with an amount of polypeptide ranging from about 10 ng to about 1 μg, and preferably about 100 ng, is sufficient to bind an adequate amount of antigen.

Covalent attachment of polypeptide to a solid support may generally be achieved by first reacting the support with a bifunctional reagent that will react with both the support and a functional group, such as a hydroxyl or amino group, on the polypeptide. For example, the polypeptide may be bound to supports having an appropriate polymer coating using benzoquinone or by condensation of an aldehyde group on the support with an amine and an active hydrogen on the polypeptide (see, e.g., Pierce Immunotechnology Catalog and Handbook, 1991, at A12-A13).

In certain embodiments, the assay is an enzyme linked immunosorbent assay (ELISA). This assay may be performed by first contacting a polypeptide antigen that has been immobilized on a solid support, commonly the well of a microtiter plate, with the sample, such that antibodies to the polypeptide within the sample are allowed to bind to the immobilized polypeptide. Unbound sample is then removed from the immobilized polypeptide and a detection reagent capable of binding to the immobilized antibody-polypeptide complex is added. The amount of detection reagent that remains bound to the solid support is then determined using a method appropriate for the specific detection reagent.

More specifically, once the polypeptide is immobilized on the support as described above, the remaining protein binding sites on the support are typically blocked. Any suitable blocking agent known to those of ordinary skill in the art, such as bovine serum albumin (BSA) or Tween 20™ (Sigma Chemical Co., St. Louis, Mo.) may be employed. The immobilized polypeptide is then incubated with the sample, and antibody is allowed to bind to the antigen. The sample may be diluted with a suitable diluent, such as phosphate-buffered saline (PBS) prior to incubation. In general, an appropriate contact time (i.e., incubation time) is that period of time that is sufficient to detect the presence of antibody within a B. microti-infected sample. Preferably, the contact time is sufficient to achieve a level of binding that is at least 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. Detection reagent may then be added to the solid support. An appropriate detection reagent is any compound that binds to the immobilized antibody-polypeptide complex and that can be detected by any of a variety of means known to those in the art. Preferably, the detection reagent contains a binding agent (such as, for example, Protein A, Protein G, immunoglobulin, lectin or free antigen) conjugated to a reporter group. Preferred reporter groups include enzymes (such as horseradish peroxidase), substrates, cofactors, inhibitors, dyes, radionuclides, luminescent groups, fluorescent groups and biotin. The conjugation of binding agent to reporter group may be achieved using standard methods known to those of ordinary skill in the art. Common binding agents may also be purchased conjugated to a variety of reporter groups from many commercial sources (e.g., Zymed Laboratories, San Francisco, Calif., and Pierce, Rockford, Ill.).

The detection reagent is then incubated with the immobilized antibody-polypeptide complex for an amount of time sufficient to detect the bound antibody. An appropriate amount of time may generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of anti-B. microti antibodies in the sample, the signal detected from the reporter group that remains bound to the solid support is generally compared to a signal that corresponds to a predetermined cut-off value. In one preferred embodiment, the cut-off value is the average mean signal obtained when the immobilized antigen is incubated with samples from an uninfected patient. In general, a sample generating a signal that is three standard deviations above the predetermined cut-off value is considered positive for babesiosis. In an alternate preferred embodiment, the cut-off value is determined using a Receiver Operator Curve, according to the method of Sackett et al., Clinical Epidemiology: A Basic Science for Clinical Medicine, Little Brown and Co., 1985, pp. 106-107. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for babesiosis.

In a related embodiment, the assay is performed in a rapid flow-through or strip test format, wherein the antigen is immobilized on a membrane, such as nitrocellulose. In the flow-through test, antibodies within the sample bind to the immobilized polypeptide as the sample passes through the membrane. A detection reagent (e.g., protein A-colloidal gold) then binds to the antibody-polypeptide complex as the solution containing the detection reagent flows through the membrane. The detection of bound detection reagent may then be performed as described above. In the strip test format, one end of the membrane to which polypeptide is bound is immersed in a solution containing the sample. The sample migrates along the membrane through a region containing detection reagent and to the area of immobilized polypeptide. Concentration of detection reagent at the polypeptide indicates the presence of anti-B. microti antibodies in the sample. Typically, the concentration of detection reagent at that site generates a pattern, such as a line, that can be read visually. The absence of such a pattern indicates a negative result. In general, the amount of polypeptide immobilized on the membrane is selected to generate a visually discernible pattern when the biological sample contains a level of antibodies that would be sufficient to generate a positive signal in an ELISA, as discussed above. Preferably, the amount of polypeptide immobilized on the membrane ranges from about 25 ng to about 1 pg, and more preferably from about 50 ng to about 500 ng. Such tests can typically be performed with a very small amount (e.g., one drop) of patient serum or blood.

Of course, numerous other assay protocols exist that are suitable for use with the polypeptides and antigenic epitopes of the present invention. The above descriptions are intended to be exemplary only.

In yet another aspect, the present invention provides antibodies to the polypeptides and antigenic epitopes of the present invention. Antibodies may be prepared by any of a variety of techniques known to those of ordinary skill in the art. See, e.g., Harlow and Lane, Antibodies: A Laboratory Manual, Cold Spring Harbor Laboratory, Cold Spring Harbor, N.Y., 1988. In one such technique, an immunogen comprising the antigenic polypeptide or epitope is initially injected into any of a wide variety of mammals (e.g., mice, rats, rabbits, sheep and goats). The polypeptides and antigenic epitopes of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide or antigenic epitope may then be purified from such antisera by, for example, affinity chromatography using the polypeptide or antigenic epitope coupled to a suitable solid support.

Monoclonal antibodies specific for the antigenic polypeptide or epitope of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide or antigenic epitope of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and tested for binding activity against the polypeptide or antigenic epitope. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides or antigenic epitopes of this invention may be used in the purification process in, for example, an affinity chromatography step.

Antibodies may be used in diagnostic tests to detect the presence of B. microti antigens using assays similar to those detailed above and other techniques well known to those of skill in the art, thereby providing a method for detecting B. microti infection in a patient.

The presence of B. microti infection may also, or alternatively, be detected based on the level of mRNA encoding a B. microti-specific protein in a biological sample. For example, at least two oligonucleotide primers may be employed in a polymerase chain reaction (PCR) based assay to amplify a portion of a B. microti-specific cDNA derived from a biological sample, wherein at least one of the oligonucleotide primers is specific for (i.e., hybridizes to) a polynucleotide encoding the B. microti protein. The amplified cDNA is then separated and detected using techniques well known in the art, such as gel electrophoresis. Similarly, oligonucleotide probes that specifically hybridize to a polynucleotide encoding a B. microti protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a B. microti protein that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule that is complementary to polynucleotide disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

Primers or probes may thus be used to detect B. microti-specific sequences in biological samples, preferably sputum, blood, serum, saliva, cerebrospinal fluid or urine. Oligonucleotide primers and probes may be used alone or in combination with each other.

In another aspect, the present invention provides methods for using one or more of the above polypeptides, antigenic epitopes or fusion proteins (or polynucleotides encoding such polypeptides) to induce protective immunity against B. microti infection in a patient. As used herein, a “patient” refers to any warm-blooded animal, preferably a human. A patient may be afflicted with a disease, or may be free of detectable disease and/or infection. In other words, protective immunity may be induced to prevent or treat babesiosis.

In this aspect, the polypeptide, antigenic epitope, fusion protein or polynucleotide is generally present within a pharmaceutical composition, or a vaccine or immunogenic composition. Pharmaceutical compositions may comprise one or more polypeptides, each of which may contain one or more of the above sequences (or variants thereof), and a physiologically acceptable carrier. Vaccines, or immunogenic compositions may comprise one or more of the above polypeptides and an immunostimulant, such as an adjuvant or a liposome (into which the polypeptide is incorporated). Such pharmaceutical compositions and immunogenic compositions may also contain other B. microti antigens, either incorporated into a combination polypeptide or present within a separate polypeptide.

Alternatively, an immunogenic composition may contain a polynucleotide encoding one or more polypeptides, antigenic epitopes or fusion proteins as described above, such that the polypeptide is generated in situ. In such immunogenic compositions, the polynucleotide may be present within any of a variety of delivery systems known to those of ordinary skill in the art, including nucleic acid expression systems, bacterial and viral expression systems. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a preferred embodiment, the polynucleotide may be introduced using a viral expression system (e.g., vaccinia or other pox virus, retrovirus, or adenovirus), which may involve the use of a non-pathogenic (defective), replication competent virus. Techniques for incorporating polynucleotides into such expression systems are well known to those of ordinary skill in the art. The polynucleotide may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In a related aspect, a DNA vaccine as described above may be administered simultaneously with or sequentially to either a polypeptide of the present invention or a known B. microti antigen. For example, administration of a polynucleotide encoding a polypeptide of the present invention, either “naked” or in a delivery system as described above, may be followed by administration of an antigen in order to enhance the protective immune effect of the vaccine, or immunogenic composition.

Routes and frequency of administration, as well as dosage, will vary from individual to individual. In general, the pharmaceutical compositions and immunogenic compositions may be administered by injection (e.g., intracutaneous, intramuscular, intravenous or subcutaneous), intranasally (e.g., by aspiration) or orally. Between 1 and 3 doses may be administered for a 1-36 week period. Preferably, 3 doses are administered, at intervals of 3-4 months, and booster vaccinations may be given periodically thereafter. Alternate protocols may be appropriate for individual patients. A suitable dose is an amount of polypeptide or polynucleotide that, when administered as described above, is capable of raising an immune response in an immunized patient sufficient to protect the patient from B. microti infection for at least 1-2 years. In general, the amount of polypeptide present in a dose (or produced in situ by the polynucleotide in a dose) ranges from about 1 pg to about 1100 mg per kg of host, typically from about 10 pg to about 1 mg, and preferably from about 100 pg to about 1 μg. Suitable dose sizes will vary with the size of the patient, but will typically range from about 0.1 mL to about 5 mL.

While any suitable carrier known to those of ordinary skill in the art may be employed in the pharmaceutical compositions of this invention, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of adjuvants may be employed in the immunogenic compositions of this invention to enhance the immune response. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants. In certain embodiments, the inventive immunogenic compositions include an adjuvant capable of eliciting a predominantly Th-1 type response. Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corp. (Hamilton, Mont.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555 and WP 99/33488. Immunostimulatory DNA sequences are also described, for example, by Sato et al., Science 273:352, 1996. Another preferred adjuvant is a saponin, preferably QS21 (Aquila, United States), which may be used alone or in combination with other adjuvants. For example, an enhanced system involves the combination of a monophosphoryl lipid A and saponin derivative, such as the combination of Qr21 and 3D-MPL as described in WO 94/00153, or a less reactogenic composition where the QS21 is quenched with cholesterol, as described in WO 96/33739. Other preferred formultions comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Corixa, Hamilton, Mont.), RC-529 (Corixa, Hamilton, Mont.) To and other aminoalkyl glucosaminide 4-phosphates (AGPs), such as those described in pending U.S. patent application Ser. Nos. 08/853,826 and 09/074,720, the disclosures of which are incorporated herein by reference in their entireties.

The following Examples are offered by way of illustration and not by way of limitation.

### EXAMPLE 1

### Isolation of DNA Sequences Encoding B. microti Antigens

This example illustrates the preparation of DNA sequences encoding B. microti antigens by screening a B. microti expression library with sera obtained from patients infected with B. microti.

B. microti genomic DNA was isolated from infected hamsters and sheared by sonication. The resulting randomly sheared DNA was used to construct a B. microti genomic expression library (approximately 0.5-4.0 kbp inserts) with EcoRI adaptors and a Lambda ZAP II/EcoRI/CIAP vector (Stratagene, La Jolla, Calif.). The unamplified library (1.2×10⁶/ml) was screened with an E. coli lysate-absorbed B. microti patient serum pool, as described in Sambrook et al., Molecular Cloning: A Laboratory Manual, Cold Spring Harbor Laboratories, Cold Spring Harbor, N.Y., 1989. Positive plaques were visualized and purified with goat-anti-human alkaline phosphatase. Phagemid from the plaques was rescued and DNA sequence for positive clones was obtained using forward, reverse, and specific internal primers on a Perkin Elmer/Applied Biosystems Inc. Automated Sequencer Model 373A (Foster City, Calif.).

Seventeen antigens (hereinafter referred to as BMNI-1-BMNI-17) were purified and three were possibly redundant. The determined DNA sequences for BMNI-1-BMNI-17 are shown in SEQ ID NOs:1-17, respectively. The deduced amino acid sequences for BMNI-1-BMNI-6, BMNI-8 and BMNI-10-BMNI-17 are shown in SEQ ID NOs:18-32, respectively, with the predicted 5′ and 3′ protein sequences for BMNI-9 being shown in SEQ ID NOs:33 and 34, respectively.

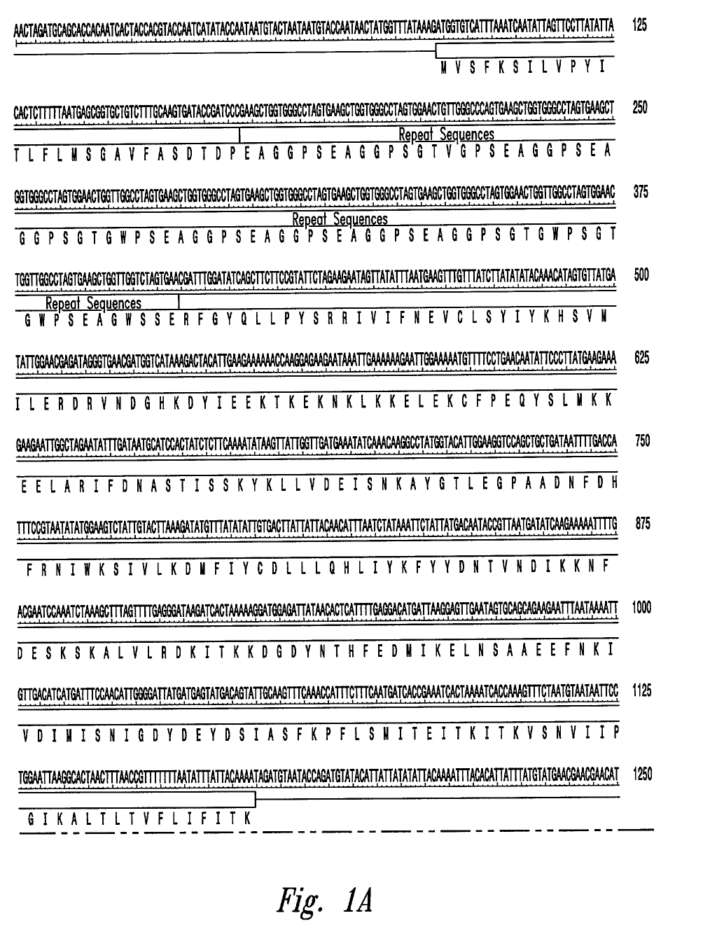

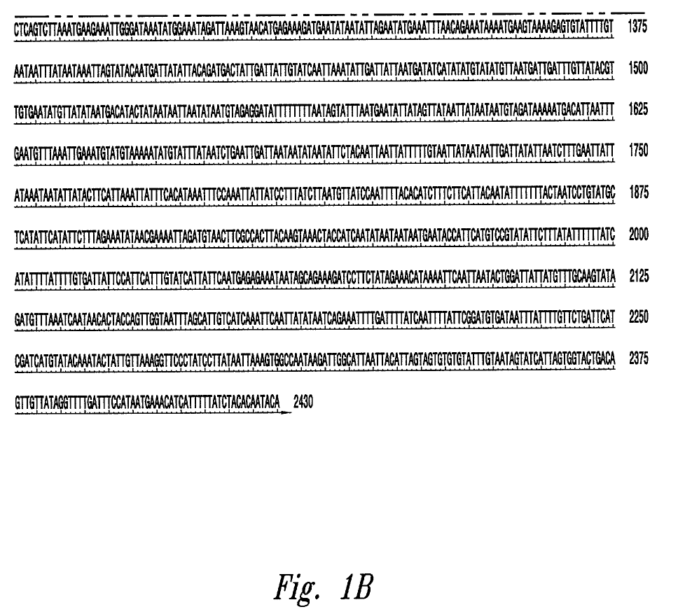

The isolated DNA sequences were compared to known sequences in the gene bank using the DNA STAR system. Nine of the seventeen antigens (BMNI-1, BMNI-2, BMNI-3, BMNI-5, BMNI-6, BMNI-7, BMNI-12, BMNI-13 and BMNI-16) share some homology, with BMNI-1 and BMNI-16 being partial clones of BMNI-3. All of these nine antigens contain a degenerate repeat of six amino acids (SEQ ID NO:35), with between nine to twenty-two repeats occurring in each antigen. The repeat portion of the sequences was found to bear some similarity to a Plasmodium falciparum merozoite surface antigen (MSA-2 gene). FIG. 1 shows the genomic sequence of BMNI-3 including a translation of the putative open reading frame, with the internal six amino acid repeat sequence being indicated by vertical lines within the open reading frame.

A second group of five antigens bear some homology to each other but do not show homology to any previously identified sequences (BMNI-4, BMNI-8, BMNI-9, BMNI-10 and BMNI-11). These antigens may belong to a family of genes or may represent parts of a repetitive sequence. BMNI-17 contains a novel degenerate repeat of 32 amino acids (SEQ ID NO:36). Similarly, the reverse complement of BMNI-17 (SEQ ID NO:37) contains an open reading frame that encodes an amino acid sequence (SEQ ID NO:38) having a degenerate 32 amino acid repeat (SEQ ID NO:39).

The reverse complement of BMNI-3 (SEQ ID NO:40) has an open reading frame which shows homology with the BMNI-4-like genes. The predicted amino acid sequence encoded by this open reading frame is shown in SEQ ID NO:41. The reverse complement of BMNI-5 (SEQ ID NO:42) contains a partial copy of a BMNI-3-like sequence and also an open reading frame with some homology to two yeast genes (S. cerevisiae G9365 ORE gene, and S. cerevisiae accession no. U18922). The predicted 5′ and 3′ amino acid sequences encoded by this open reading frame are shown in SEQ ID NOs:43 and 44, respectively. The reverse complement of BMNI-7 (SEQ ID NO: 45) contains an open reading frame encoding the amino acid sequence shown in SEQ ID NO:46.

A telomeric repeat sequence, which is conserved over a wide range of organisms, was found in five antigens (BMNI-2, BMNI-5, BMNI-6, BMNI-7 and BMNI-16), indicating that many of the isolated genes may have a telomere-proximal location in the genome. BMNI-10 appears to include a double insert, the 3′-most segment having some homology to E. coli aminopeptidase N. In addition, BMNI-7 contains apparently random insertions of hamster DNA. One such insertion has characteristics of a transposible element (i.e. poly A tail and flanked by a direct repeat).

In subsequent studies, two additional B. microti antigens were isolated by screening the B. microti genomic DNA expression library described above with a serum pool from B. microti infected patients that showed low reactivity with recombinant proteins generated from clones BMNI-2-BMNI-17. The determined DNA sequences for these two clones, hereinafter referred to as MN-10 and BMNI-20, are provided in SEQ ID NOs:50 and 51, respectively, with the corresponding predicted amino acid sequences being provided in SEQ ID NOs:52 and 53. MN-b was found to extend the sequence of BMNI-4 in the 3′ direction and BMNI-20 was found to extend the sequence of BMNI-17 in the 5′ direction.

Additional B. microti sequences were identified using a technique designed to target secreted or shed antigens. Specifically, infection with B. microti (strain MN1) was established by intraperitoneal inoculation of 500 ul of cyropreserved hamster blood into 3 week old 50 g female Golden Syrian hamsters (SASCO; Charles River, Wilmington, Mass.). Infection was monitored by use of Giemsa-stained or acridine orange-stained blood smear over a 2 week period. Blood was harvested by cardiac puncture when the parasitemia levels reached 60-70%. Infected blood was diluted in saline to 100,000,000 infected red blood cells/mL. This blood was then used to inoculate several CB-17 SCID mice (Jackson Labs, Bar Harbor, Me.). Infection was monitored as above. At 3 weeks post-inoculation, the blood was harvested and had a parasitemia of approx. 5%. Serum was obtained by centrifuging the harvested blood at approx. 3000 rpm for 5-10 minutes and removing the serum from the top of the pelleted cells and debris. Syngeneic immunocompetent mice (BALB/c) were immunized with 200 ul total of a 1:1 (vol:vol) mixture of the SCID sera and MPL adjuvant monthly for a total of 5 injections. The BALB/c mice were bled via the tail vein 12 days post-3^{rd} and 4^{th} immunizations and were bled via cardiac stick post-5^{th} immunization.

The serum was used to screen the B. microti expression library described above for secreted/shed antigens. Before screening, the serum was adsorbed with E. coli proteins on nitrocellulose filters. The library was plated on eleven large Petri plates at a concentration of approximately 20,000 plaques/plate. The plaques were lifted onto nitrocellulose filters and then processed using standard protocols with the adsorbed SCID sera as the primary antibody and goat anti-mouse (IgGT, IgA, IgM HPL), alkaline phosphatase conjugated, secondary antibody to visualize positive plaques.

Seventy plaques were picked upon the first screening of the library. These plaques were then processed and replated for secondary screens and, in some cases, tertiary screens. Twenty-seven clones were confirmed as positive and processed according to the protocols developed by Stratagene for their ZAP II vector for excision of the insert and subsequently cloning into the SOLR strain of E. coli (Stratagene, La Jolla, Calif.). The DNA from the inserts in each clone was sequenced in both directions. The 5′ cDNA sequence for clone BM10 is provided in SEQ ID NO:91, the 5′ and 3′ cDNA sequences for clone BM12 are provided in SEQ ID NOs: 92 and 93, respectively; the 5′ and 3′ cDNA sequences for clone BM21 are provided in SEQ ID NOs:94 and 95, respectively; the 5′ and 3′ cDNA sequences for clone BM24 are provided in SEQ ID NOs:96 and 97, respectively; the 5′ cDNA sequence for clone BM26 is provided in SEQ ID NO:98; the complete cDNA sequence for the insert of clone BM31 is provided in SEQ ID NO:99; the 5′ and 3′ cDNA sequences for clone BM33 are provided in SEQ ID NOs:100 and 101, respectively; the 3′ cDNA sequence for clone BM37 is provided in SEQ ID NO: 102; the complete cDNA sequence for a BMNI-10 clone is provided in SEQ ID NO:103; the complete cDNA sequence for the insert of clone BM61 is provided in SEQ ID NO:104; the 3′ cDNA sequence for clone BM6.36 is provided in SEQ ID NO: 105; the complete cDNA sequence for the insert of clone BM4 is provided in SEQ ID NO:106; the complete cDNA sequence for the insert of clone BM45 is provided in SEQ ID NO:107; the complete cDNA sequence for the insert of clone BM40.42 is provided in SEQ ID NO: 108; the complete cDNA sequence for a BMNI-11-like clone (referred to as BM111) is provided in SEQ ID NO: 109; and the complete cDNA sequence for a BMNI-15-like clone (referred to as BM15) is provided in SEQ ID NO: 110.

The sequences of SEQ ID NOs:96, 99, 101 and 104 were found to show some similarity to sequences previous deposited in Genbank and/or GeneSeq. The sequences of SEQ ID NOs:107 and 110 were found to have some overlap. SEQ ID NO:105 was found to show some similarity to the sequence of MN10 described above. The sequences of SEQ ID NOs: 103, 109 and 110 were found to show some similarity to the sequences of BMNI-10, BMNI-11 and BMNI-15 described above. No significant similarities were found to the sequences of SEQ ID NOs:91-95, 97, 98, 100, 102, 106 and 108.

Subsequent studies led to the isolation of extended cDNA sequences for the clones BM61, BM6.36, BM4, BM45, BM31, BM26, BM15, BM12 and BM11 (SEQ ID NO: 111-119, respectively) and to the isolation of an additional clone, referred to as BM28 (SEQ ID NO: 135), which was found to be related to BM11. Each of these sequences was found to contain an open reading frame. The amino acid sequences encoded by the extended cDNA sequences of BM15, BM11, BM61, BM6.36, BM12, BM26, BM31 and BM4 are provided in SEQ ID NO: 120-127, respectively, with the amino acid sequence for BM28 being provided in SEQ ID NO: 136. The amino acid sequence encoded by the ORF of BM45 is provided in SEQ ID NO: 134, and is contained within SEQ ID NO: 120. Extended cDNA sequences for the clones BM33, BM21, BM24 and BM40.42 are provided in SEQ ID NO: 128-131, respectively. The amino acid sequences encoded by the cDNA sequences for BM40.42, and BM24 are provided in SEQ ID NO: 132 and 133, respectively.

### EXAMPLE 2

### Synthesis of Synthetic Polypeptides

Polypeptides may be synthesized on a Millipore 9050 peptide synthesizer using FMOC chemistry with HPTU (O-Benzotriazole-N,N,N′,N′-tetramethyluronium hexafluorophosphate) activation. A Gly-Cys-Gly sequence may be attached to the amino terminus of the peptide to provide a method of conjugating or labeling of the peptide. Cleavage of the peptides from the solid support may be carried out using the following cleavage mixture: trifluoroacetic acid:ethanedithiol:thioanisole:water:phenol (40:1:2:2:3). After cleaving for 2 hours, the peptides may be precipitated in cold methyl-t-butyl-ether. The peptide pellets may then be dissolved in water containing 0.1% trifluoroacetic acid (TFA) and lyophilized prior to purification by C18 reverse phase HPLC. A gradient of 0-60% acetonitrile (containing 0.1% TFA) in water (containing 0.1% TFA) may be used to elute the peptides. Following lyophilization of the pure fractions, the peptides may be characterized using electrospray mass spectrometry and by amino acid analysis.

This procedure was used to synthesize two peptides (hereinafter referred to as BABS-1 and BABS-4) made to the repeat region of the isolated B. microti antigen BMNI-3. The sequences of BABS-1 and BABS-4 are shown in SEQ ID NO: 47 and 48, respectively.

### EXAMPLE 3

### Use of Representative Antigens and Peptides for Serodiagnosis of B. microti Infection

The diagnostic properties of recombinant BMNI-3, BMNI-4, BMNI-6, BMNI-15, MN-b and BMNI-20, and the BABS-l and BABS-4 peptides were determined as follows.

Assays were performed in 96 well plates coated overnight at 4° C. with 200 ng antigen/well added in 50 μl of carbonate coating buffer. The plate contents were then removed and the wells were blocked for 2 hours with 200 μl of PBS/1% BSA. After the blocking step, the wells were washed six times with PBS/0.1% Tween 20™ Fifty microliters of sera, diluted 1:100 in PBS/0.1% Tween 20™/0.1% BSA, was then added to each well and incubated for 30 minutes at room temperature. The plates were then washed six times with PBS/0.1% Tween 20™.

The enzyme conjugate (horseradish peroxidase-Protein A, Zymed, San Francisco, Calif.) was then diluted 1:20,000 in PBS/0.1% Tween 20™/0.1% BSA, and 50 μl of the diluted conjugate was added to each well and incubated for 30 minutes at room temperature. Following incubation, the wells were washed six times with PBS/0.1% Tween 20™. 100 μl of tetramethylbenzidine peroxidase substrate (Kirkegaard and Perry Laboratories, Gaithersburg, Md.) was added, undiluted, and incubated for 15 minutes. The reaction was stopped by the addition of 100 μl of 1N H₂SO₄ to each well and the plates were read at 450 nm.

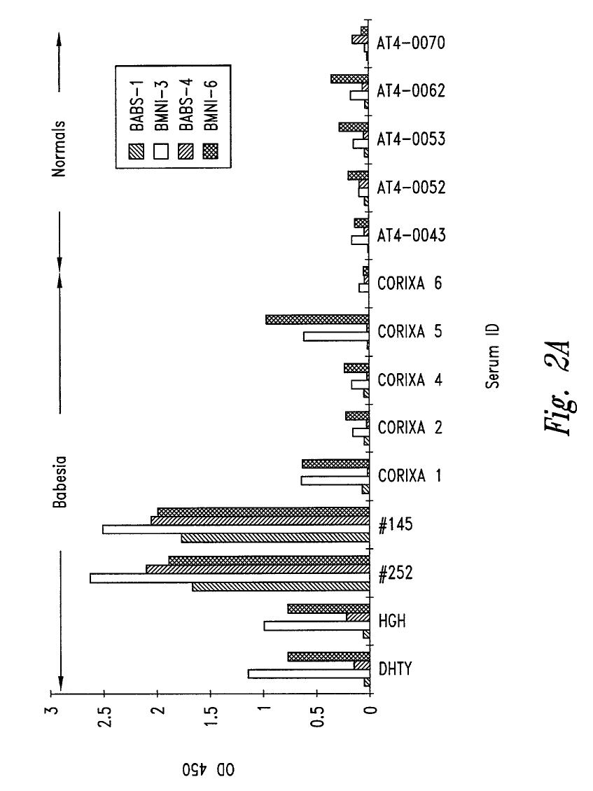

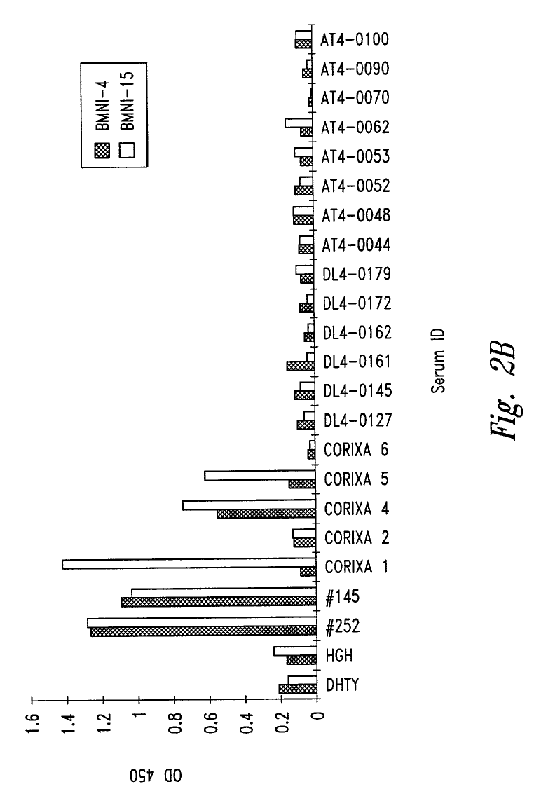

FIG. 2a shows the reactivity of the recombinant BMNI-3 and BMNI-6 antigens and the two peptides BABS-l and BABS-4 in the ELISA assay. The recombinant antigens and the two peptides were negative in ELISA with all seven samples from normal (B. microti negative) individuals. In contrast, both BMNI-3 and BMNI-6 detected six of the nine B. microti-infected samples, as compared to two out of the nine for the BABS-1 and BABS-4 peptides. This would suggest that BMNI-3 and BMNI-6 may contain other antigenic epitopes in addition to those present in the repeat epitopes in BABS-1 and BABS-4, or that an insufficient number of repeats are available in the peptides to fully express the antigenic epitopes present in the recombinant antigens BMNI-3 and BMNI-6.

FIG. 2b shows the ELISA reactivity of the recombinant antigens BMNI-4 and BMNI-15. Both recombinants were negative with all fifteen samples from normal individuals. BMNI-4 detected four out of nine B. microti-infected samples and BMNI-15 detected six out of nine B. microti-infected samples. Both BMNI-4 and BMNI-15 detected a B. microti-infected sample which was not detected by BMNI-3 or BMNI-6, suggesting that BMNI-4 and BMNI-15 might be complementary to BMNI-3 and BMNI-6 in the ELISA test described herein.

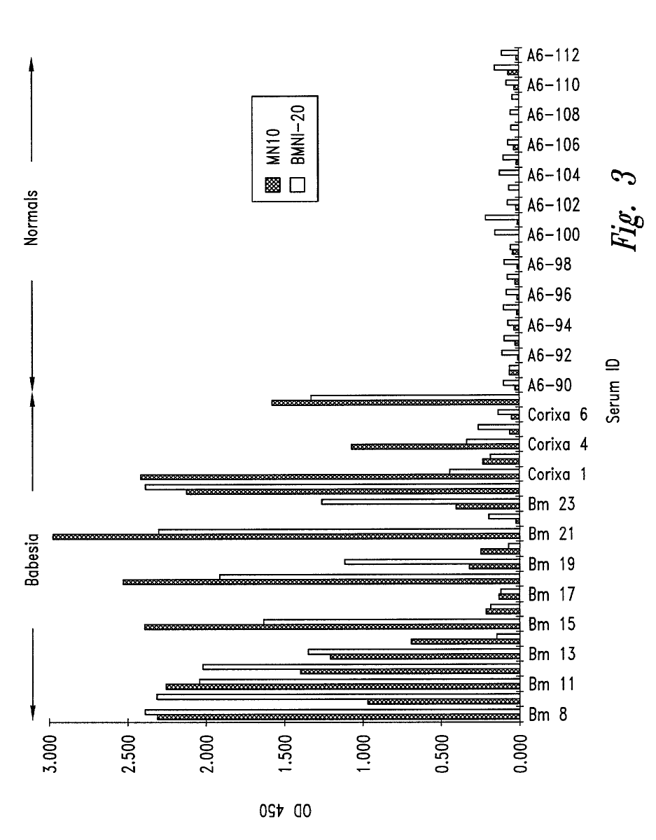

The ELISA reactivity of recombinant MN-10 and BMNI-20 with sera from B. microti-infected patients and from normal donors is shown in FIG. 3. MN-10 and BMNI-20 were found to be reactive with B. microti-infected sera that were not reactive with recombinant BMNI-2 through BMNI-17. Therefore, MN-10 and BMNI-20 may be usefully employed in combination with other B. microti antigens of the present invention for the detection of B. microti infection.

Table 1 shows the reactivity of the recombinant B. microti antigens BMNI-2, BMNI-17, MN-10 and a combination of BMNI-17 and MN-10, as determined by ELISA, with Babesia-positive sera, sera positive for both Babesia and Ehrlichia, sera positive only for Ehrlichia, Lyme disease sera and sera from normal donors. The data indicate a sensitivity of approximately 93% and a specificity in normal donors in excess of 98%. These results indicate that a combination of BMNI-17 and MN-10 is particularly effective in the diagnosis of B. microti infection.

TABLE 1

| | | | | | Normal |
|---|---|---|---|---|---|
| Antigen | Babesia | Babesia/Ehrlichia | Ehrlichia | Lyme | donors |
| BMNI-2 | 27/50 | 2/3 | 1/4 | 0/10 | 1/73 |
| BMNI-17 | 35/50 | 3/3 | 0/4 | 0/10 | 0/86 |
| MN-10 | 37/49 | 3/3 | 0/4 | 1/10 | 1/98 |
| BMNI-17/ | 46/50 | 3/3 | 0/4 | 1/10 | 1/98 |
| MN-10 | | | | | |

B. Diagnostic Properties of Representative Antigens and Peptides as Determined by Western Analysis

Western blot analyses were performed on representative B. microti antigens as follows.

Antigens were induced as pBluescript SK-constructs (Stratagene), with 2 mM IPTG for three hours (T3), after which the resulting proteins from time 0 (T0) and T3 were separated by SDS-PAGE on 15% gels. Separated proteins were then transferred to nitrocellulose and blocked for 1 hr in 0.1% Tween 20™/PBS. Blots were then washed 3 times in 0.1% Tween 20™/PBS and incubated with a B. microti patient serum pool (1:200) for a period of 2 hours. After washing blots in 0.1% Tween 20™/PBS 3 times, immunocomplexes were detected by the addition of Protein A conjugated to ¹²⁵I (1/25000; NEN-Dupont, Billerica, Mass.) followed by exposure to X-ray film (Kodak XAR 5; Eastman Kodak Co., Rochester, N.Y.) at −70° C. for 1 day.

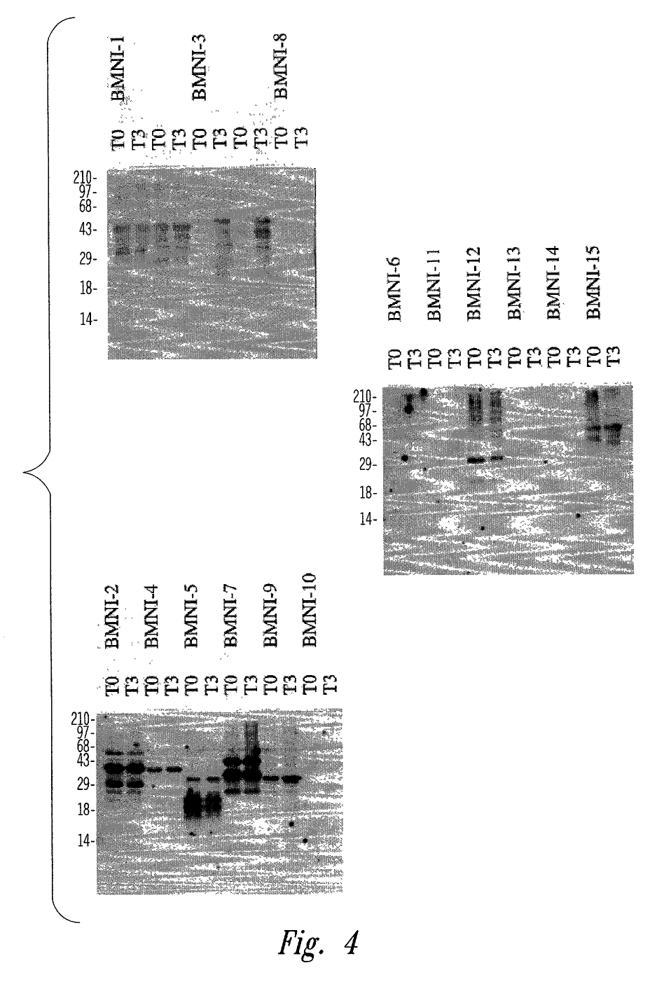

As shown in FIG. 4, resulting bands of reactivity with serum antibody were seen at 43 kDa for BMNI-1, 38 kDa for BMNI-2, 45 kDa for BMNI-3, 37 kDa for BMNI-4, 18 and 20 kDa for BMNI-5, 35 and 43 kDa for BMNI-7, 32 kDa for BMNI-9, 38 kDa for BMNI-11. 30 kDa for BMNI-12, 45 kDa for BMNI-15, and 43 kDa for BMNI-17 (not shown). Antigen BMNI-6, after reengineering as a pET 17b construct (Novagen, Madison, Wis.) showed a band of reactivity at 33 kDa (data not shown). Protein size standards, in kDa (Gibco BRL, (Gaithersburg, Md.), are shown to the left of the blots.

Western blots were performed on purified BMNI-3, BMNI-2, BMNI-15, BMNI-17 and MN-10 recombinant antigen with a series of patient sera from B. microti patients and from patients with either Lyme disease or ehrlichiosis. Specifically, purified recombinant antigen (4 μg) was separated by SDS-PAGE on 12% gels. Protein was then transferred to nitrocellulose membrane for immunoblot analysis. The membrane was first blocked with PBS containing 1% Tween 20™ for 2 hours. Membranes were then cut into strips and incubated with individual sera (1/500) for two hours. The strips were washed 3 times in PBS/0.1% Tween 20™ containing 0.5 M NaCl prior to incubating with Protein A-horseradish peroxidase conjugate (1/20,000) in PBS/0.1% Tween 20™/0.5 M NaCl for 45 minutes. After further washing three times in PBS/0.1% Tween 20 ™/0.5 M NaCl, ECL chemiluminescent substrate (Amersham, Arlington Heights, Ill.) was added for 1 min. Strips were then reassembled and exposed to Hyperfilm ECL (Amersham) for 5-30 seconds.

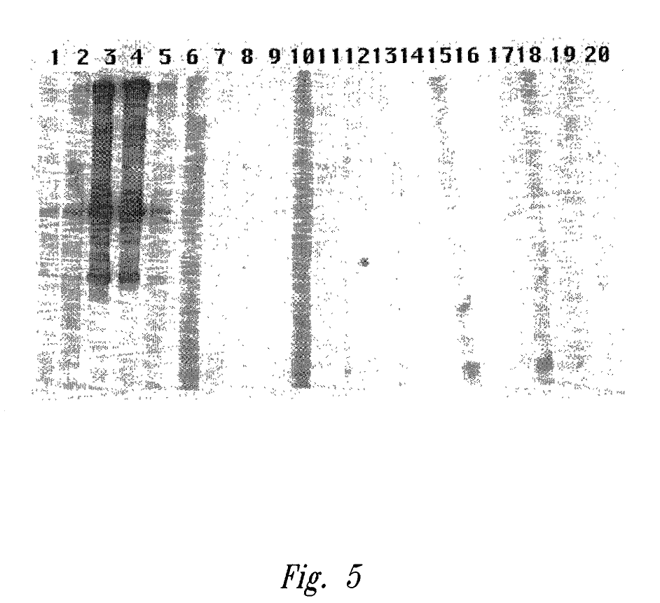

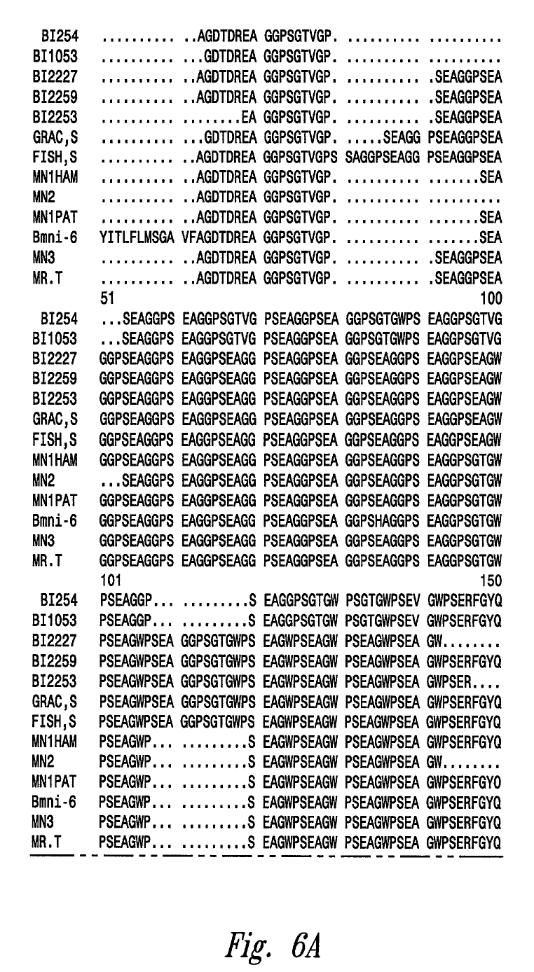

Lanes 1-9 of FIG. 5 show the reactivity of purified recombinant BMNI-3 with sera from nine B. microti-infected patients, of which five were clearly positive and a further two were low positives detectable at higher exposure to the hyperfilm ECL. This correlates with the reactivity as determined by ELISA. In contrast, no immunoreactivity was seen with sera from patients with either ehrlichiosis (lanes 10 and 11) or Lyme disease (lanes 12-14), or with sera from normal individuals (lanes 15-20). A major reactive band appeared at 45 kDa and a small break down band was seen at approximately 25 kDa.

Table 2, below, summarizes the reactivity of the recombinant antigens BMNI-2, BMNI-15, BMNI-17 and MN-10 with B. microti positive sera. No reactivity was seen with Lyme or Ehrlichia-infected sera, with little or no reactivity being seen with normal sera.

TABLE 2

| Sample ID | BMNI-2 | BMNI-15 | BMNI-17 | MN-10 |
|---|---|---|---|---|
| BM8 | ++ | ++ | +++++ | − |
| BM21 | ++ | − | ++++ | ++++ |
| COR4 | ± | ++++ | ++++ | + |
| COR5 | ± | +++ | + | − |
| 252 | ++++ | ++++ | ++++++ | +++ |

−indicates no reactivity

### EXAMPLE 4

### Analysis of Geographic Variation Within Antigens

The reactivity of the inventive antigens with sera from B. microti patients, as determined by Western blot, was found to vary with the U.S. location of the patients. Accordingly, geographic variation within the gene encoding the exemplary antigen BMNI-6 was examined as follows.

Two PCR primers, referred to as BMNI-6/5′ and BMNI-6/3′ (SEQ ID NOs:54 and 55, respectively) were designed based on the region flanking the six amino acid degenerate repeat region of BMNI-6 (SEQ ID NO:6). These primers were employed to amplify genomic DNA from whole blood obtained from twelve B. microti-infected patients and genomic DNA from whole blood from P. leucopus and hamsters in a Perkin Elmer 480 thermal cycler using the manufacturer's protocol. PCR products were evaluated for size on 2% agarose gels and then Southern blotted and probed with a DIG-labeled oligonucleotide. Positive clones were sequenced using an Applied Biosystems Model 373A or 377 sequencer. RT-PCR was performed on Trizol LS extracted B. microti-infected hamster whole blood RNA using the primers described above, and the resulting clones were sequenced as described above.

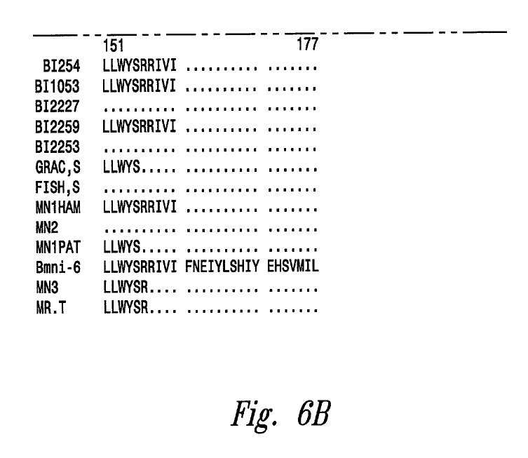

These studies resulted in the isolation of twelve BMNI-6 homologues, referred to hereinafter as BI254, BI1053, BI2227, BI2259, BI2253, BI2018, RIFS, MN1HAM, MN2, MN1PAT, MN3 and MRT with MN1HAM being obtained from hamster and the other eleven from patients. The determined DNA sequences of these clones are provided in SEQ ID NO:56-67, respectively, with the corresponding predicted amino acid sequences being provided in SEQ ID NO:68-79, respectively. Isolates from hamsters had the same sequences as found in the corresponding human blood, suggesting that genetic variation of BMNI-6 does not occur during passage. However, clones from different patients often showed variation in the number and location of the degenerate repeat found within BMNI-6. An alignment of the repeat regions from each of the twelve clones is provided in FIG. 6. Furthermore, strains that were closely related geographically were also closely related at the sequence level. For example, three patients from Nantucket Island, Mass., harbored clones (BI2253, BI12259 and BI2227) that were indistinguishable from each other but distinct from those found in other northeastern or upper midwestern strains. These results suggest that considerable antigenic diversity exists among isolates of B. microti from the U.S. and that geographic clustering of subtypes exists.

### EXAMPLE 5

### Preparation and Characterization of B. microti Fusion Proteins

A. Preparation of a Fusion Protein Containing MN-10 and BMNI-17

A fusion protein containing the B. microti antigens MN-10 and BMNI-17, referred to as BaF-3, was prepared as follows.

MN-10 and BMNI-17 DNA was used to perform PCR using the primers PDM-285 and PDM-286 (SEQ ID NOs:80 and 81); and PDM-283 and PDM-284 (SEQ ID NOs:82 and 83), respectively. In both cases, the DNA amplification was performed using 10 μl of 10×Pfu buffer (Stratagene), 1 μl of 10 mM dNTPs, 2 μl each of the PCR primers at 10 μM concentration, 83 μl water, 1.5 μl Pfu DNA polymerase (Stratagene, La Jolla, Calif.) and 1 μl DNA at 50 ng/μl. Denaturation at 96° C. was performed for 2 min, followed by 40 cycles of 96° C. for 20 sec. 59° C. for 15 sec and 72° C. for 3 min and lastly by 72° C. for 4 min. The MN-10 and BMNI-17 PCR products were digested with SspI and then ligated using a ligation kit from Panvera (Madison, Wis.). The resulting BaF-3 fusion was PCR amplified using primers PDM 285 and PDM-284 and the same conditions as listed above. This PCR product was then digested with ScaI and EcoRI, and cloned into a modified pET28 vector. The fusion construct was confirmed by sequencing. The expression construct was transformed into BL21 (DE3) CodonPlus cells (Novagen, Madison, Wis.) for induction and expression. The protein came out in the inclusion body pellet. This pellet was washed three times with a 0.5% CHAPS wash in 20 mM Tris (8.0) and 300 mM NaCl. The pellet was then solubilized in 8 M urea, 20 mM Tris (8.0), 300 mM NaCl and batch bound to Nickel NTA resin (Qiagen). The nickel resin was washed with 100 ml 8 M urea, 20 mM Tris (9.0), 300 mM NaCl, 1% DOC. A second wash was performed as described for the first wash, but with the omission of DOC. The protein was first eluted with 8 M urea, 20 mM Tris (9.0), 100 mM NaCl, and 500 mM imidazole. In a second elution, the imidazole was increased to 1 M. The elutions were run on a 4-20 SDS-PAGE gel and the fractions containing the protein of interest were pooled and dialyzed against 1 mM Tris (8.).

The determined cDNA sequence of coding region for the BaF-3 fusion protein is provided in SEQ ID NO: 84, with the corresponding amino acid sequence being provided in SEQ ID NO: 85.

B. Preparation of a Fusion Protein Containing BMNI-15, MN-10 and BMNI-17

A fusion protein containing the B. microti antigens BMNI-15, MN-10 and BMNI-17, referred to as BaF-4, was prepared as follows.

BMNI-15 DNA was used to perform PCR using the primers PDM-349 and PDM-363 (SEQ ID NO: 88 and 89). DNA amplification was performed using 10 μl of 10×Pfu buffer (Stratagene), 1 μl of 10 mM dNTPs, 2 μl each of the PCR primers at 10 μM concentration, 83 μl water, 1.5 μl Pfu DNA polymerase (Stratagene, La Jolla, Calif.) and 1 μl DNA at 50 ng/μl. Denaturation at 96° C. was performed for 2 min, followed by 40 cycles of 96° C. for 20 sec, 61° C. for 15 sec and 72° C. for 3 min, and lastly by one cycle of 72° C. for 4 min. The PCR product was digested with PvuII and EcoRI, and cloned into a modified pET28 vector, which had been cut with Eco72I and EcoRI. The construct was confirmed to be correct by sequencing. MN-10/BMNI-17 DNA from BaF-3, described above, was used to perform PCR using the primers PDM-364 and PDM-284 (SEQ ID NO: 90 and 83, respectively). DNA amplification was performed using 10 μl of 10×Pfu buffer (Stratagene), 1 μl of 10 mM dNTPs, 2 μl each of the PCR primers at 10 μM concentration, 83 μl water, 1.5 μl Pfu DNA polymerase (Stratagene, La Jolla, Calif.) and 1 μl DNA at 50 ng/μl. Denaturation at 96° C. was performed for 2 min, followed by 40 cycles of 96° C. for 20 sec, 60° C. for 15 sec and 72° C. for 6 min, and lastly by 72° C. for 4 min. The PCR product was cut with BamHI and EcoRI, and cloned into the pPDM BMNI-15 construct at the BamHI and EcoRI sites. The resulting construct was found by sequence analysis to have a single base pair deletion 419 bp in from the stop codon. This base pair deletion was corrected by digesting the pPDM BaF4B-6 clone with KpnI and SphI, and purifying the 2.6 kb insert plus 5′ vector. This band was then cloned into pPDM TrX2H BaF3-10 that was digested with the same enzymes and contained the 3′ end of BMNI-17 plus most of the pPDM vector. The correct sequence was confirmed by sequence analysis and then transformed into the BL21 CodonPlus expression host (Novagen).

The determined cDNA sequence of the coding region of the BaF-4 fusion protein is provided in SEQ ID NO: 86, with the corresponding amino acid sequence being provided in SEQ ID NO: 87.

C. Preparation of a Fusion Protein Containing MN-10 and BMNI-17

A fusion protein containing the B. microti antigens MN-10 and BMNI-17, referred to as BaF-5, was prepared as follows.

Two oligonucleotides referred to as PDM-391 and PDM-392 (SEQ ID NO: 137 and 138, respectively) were annealed and the resulting annealed pair was employed for ligating the linker between BMNI-17 and MN-10 at the BamHI and HindIII sites.

BMNI-17 DNA was amplified from BaF-1 by PCR using the primers PDM-252 and PDM-389 (SEQ ID NO: 139 and 140, respectively). DNA amplification was performed using 10 μl of 10×Pfu buffer (Stratagene), 1 μl of 10 mM dNTPs, 2 μl each of the PCR primers at 10 μM concentration, 83 μl water, 1.5 μl Pfu DNA polymerase (Stratagene, La Jolla, Calif.) and 50 ng DNA. Denaturation at 96° C. was performed for 2 min, followed by 40 cycles of 96° C. for 20 sec, 61° C. for 15 sec and 72° C. for 2 min, and lastly by one cycle of 72° C. for 4 min. The PCR product was digested with EcoRI and cloned into a modified pET28 vector, which had been cut with Eco72I and EcoRI. The resulting construct was referred to as pPDM BMNI-17D.

MN-10 DNA was amplified from BaF-1 by PCR using the primers PDM-252 and PDM-389 (SEQ ID NO: 141 and 142, respectively). DNA amplification was performed using 10 μl of 10×Pfu buffer (Stratagene), 1 μl of 10 mM dNTPs, 2 μl each of the PCR primers at 10 μM concentration, 83 μl water, 1.5 μl Pfu DNA polymerase (Stratagene, La Jolla, Calif.) and 50 ng DNA. Denaturation at 96° C. was performed for 2 min, followed by 40 cycles of 96° C. for 20 sec, 58° C. for 15 sec and 72° C. for 1.5 min, and lastly by one cycle of 72° C. for 4 min. The PCR product was digested with HindIII and EcoRI, and cloned into pPDM BMNI-17D.

The resulting construct was confirmed to be correct by sequencing, and then transformed into BL21 CodonPlus cells. The amino acid sequence of the BaF-5 fusion protein is provided in SEQ ID NO: 144, with the corresponding cDNA sequence for the coding region being provided in SEQ ID NO: 143.

One of skill in the art will appreciate that the order of the individual antigens within the fusion protein may be changed and that comparable or enhanced activity could be expected provided each of the epitopes is still functionally available. In addition, truncated forms of the proteins containing active epitopes may be used in the construction of fusion proteins.

### EXAMPLE 6

### Expression of Recombinant B. microti Antigens

This example describes the expression of B. microti antigens in E. coli.

A. Expression of Recombinant BMNI-15

To express the C-terminal portion of BMNI-15 (SEQ ID NO: 15) in E. coli, the open reading frame was amplified by PCR using the primers PDM-494 (SEQ ID NO: 145) and PDM-495 (SEQ JD NO: 146), and the following conditions: 10 μl of 10×Pfu buffer (Stratagene), 1 μl of 10 mM dNTPs, 2 μl each of the PCR primers at 10 μM concentration, 83 μl water, 1.5 μl Pfu DNA polymerase (Stratagene) and 50 ng DNA. Denaturation at 96° C. was performed for 2 min, followed by 40 cycles of 96° C. for 20 sec, 58° C. for 15 sec and 72° C. for 4 min and lastly by one cycle of 72° C. for 4 min. The PCR product was digested with XhoI and cloned into a modified pET28 vector with a His tag in-frame on the 5′ end, which had been digested with Eco72I and XhoI. The correct construct was confirmed through sequence analysis and transformed into BL21pLysS (Novagen, Madison, Wis.) and BLR(DE3) CodonPlus RIL E. coli cells. The amino acid sequence of the recombinant BMNI-15 C-terminal protein, including His tag, is provided in SEQ ID NO: 172, with the cDNA sequence of the coding region being provided in SEQ ID NO: 163.

To express the N-terminal portion of BMNI-15 (SEQ ID NO: 15) in E. coli, the open reading frame was amplified by PCR using the primers PDM-549 (SEQ ID NO: 147) and PDM-550 (SEQ ID NO: 148). PCR was carried out as described for the C-terminal portion of BMNI-15, except that denaturation at 96° C. for 2 mm was followed by 40 cycles of 96° C. for 20 sec, 61° C. for 15 sec and 72° C. for 1 min 20 sec. and lastly by one cycle of 72° C. for 4 min. The PCR product was digested with EcoRI and cloned into a modified pET28 vector with a His tag in-frame on the 5′ end, which had been digested with Eco72I and EcoRI. The correct construct was confirmed through sequence analysis and transformed into BLR(DE3)pLysS and BLR(DE3) CodonPlus RIL E. coli cells. The amino acid sequence of the recombinant BMNI-15 N-terminal protein, including His tag, is provided in SEQ ID NO: 173, with the cDNA sequence of the coding region being provided in SEQ ID NO: 164.

B. Expression of Recombinant BM4

To express BM4 (SEQ ID NO: 113) in E. coli, the open reading frame was amplified by PCR using the primers PDM-559 (SEQ ID NO: 149) and PDM-560 (SEQ ID NO: 150). PCR was carried out as described above for BMNI-15, except that denaturation at 96° C. for 2 min was followed by 40 cycles of 96° C. for 20 sec, 64° C. for 15 sec and 72° C. for 2 min 20 sec, and lastly by one cycle of 72° C. for 4 mm. The PCR product was digested with XhoI and cloned into a modified pET28 vector with a His tag in-frame on the 5′ end, which had been digested with Eco72I and XhoI. The correct construct was confirmed through sequence analysis and transformed into BL21pLysS and BLR(DE3) CodonPlus RIL E. coli cells. The amino acid sequence of the recombinant BM4 protein, including His tag, is provided in SEQ ID NO: 174. with the cDNA sequence of the coding region being provided in SEQ ID NO: 165.

C. Expression of Recombinant BM12

To express BM-12 (SEQ ID NO: 118) in E. coli, the open reading frame was amplified by PCR using the primers PDM-561 (SEQ ID NO: 151) and PDM-562 (SEQ ID NO: 152). PCR was carried out as described above for BMNI-15, except that denaturation at 96° C. for 2 min was followed by 40 cycles of 96° C. for 20 sec, 63° C. for 15 sec and 72° C. for 4 min, and lastly by one cycle of 72° C. for 4 min. The PCR product was digested with XhoI and cloned into a modified pET28 vector with a His tag in-frame on the 5′ end, which had been digested with Eco72I and XhoI. The correct construct was confirmed through sequence analysis and transformed into BL21pLysS and BLR(DE3) CodonPlus RIL E. coli cells. The amino acid sequence of the recombinant BM-12 protein, including His tag, is provided in SEQ ID NO: 175, with the cDNA sequence of the coding region being provided in SEQ ID NO: 166.

D. Expression of Recombinant BMNI-11

The open reading frame of BMNI-11 (SEQ ID NO: 11) starting at amino acid 10 of the signal sequence was amplified by PCR using the primers PDM-604 (SEQ ID NO: 153) and PDM-605 (SEQ ID NO: 154). PCR was carried out as described above except that denaturation for 2 min at 96° C. was followed by 40 cycles of 96° C. for 20 sec, 55° C. for 15 sec and 72° C. for 1.5 ml, and lastly by one cycle of 72° C. for 4 ml. The PCR product was digested with EcoRI and cloned into a modified pET28 vector with a His tag in-frame on the 5′ end, which had been digested with Eco72I and EcoRI. The correct construct was confirmed through sequence analysis and transformed into BLR(DE3)pLysS and BLR(DE3) CodonPlus RIL E. coli cells. The amino acid sequence of the recombinant BMNI-11 protein, including His tag, is provided in SEQ ID NO: 175, with the DNA sequence of the coding region being provided in SEQ ID NO: 167.

E. Expression of Recombinant BM61

The open reading frame of BM61 (SEQ ID NO: 111) was amplified by PCR using the primers PDM-496 (SEQ ID NO: 155) and PDM-497 (SEQ ID NO: 156). PCR was carried out as described above except that denaturation for 2 min at 96° C. was followed by 40 cycles of 96° C. for 20 sec, 61° C. for 15 sec and 72° C. for 1.5 min, and lastly by one cycle of 72° C. for 4 min. The PCR product was digested with EcoRI and cloned into a modified pET28 vector with a His tag in-frame on the 5′ end, which had been digested with Eco72I and EcoRI. The correct construct was confirmed through sequence analysis and transformed into BLR(DE3)pLysS and BLR(DE3) CodonPlus RIL E. coli cells. The amino acid sequence of the recombinant BM61 protein, including His tag, is provided in SEQ ID NO: 177, with the cDNA sequence of the coding region being provided in SEQ ID NO: 168.

F. Expression of Recombinant BM40.42

The open reading frame of BM40.42 (SEQ ID NO: 131) was amplified by PCR using the primers PDM-474 (SEQ ID NO: 157) and PDM-475 (SEQ ID NO: 158). PCR was carried out as described above except that denaturation for 2 min at 96° C. was followed by 40 cycles of 96° C. for 20 sec. 60° C. for 15 sec and 72° C. for 1 min, and lastly by one cycle of 72° C. for 4 min. The PCR product was digested with XhoI and cloned into a modified pET28 vector with a His tag in-frame on the 5′ end, which had been digested with Eco72I and XhoI. The correct construct was confirmed through sequence analysis and transformed into BL21pLysS E. coli cells. The amino acid sequence of the recombinant BM40.42 protein, including His tag, is provided in SEQ ID NO: 178, with the cDNA sequence of the coding region being provided in SEQ ID NO: 170.

G. Expression of Recombinant BM31

The open reading frame of BM31 (SEQ ID NO: 115) was amplified by PCR using the primers PDM-606 (SEQ ID NO: 159) and PDM-607 (SEQ ID NO: 160). PCR was carried out as described above except that denaturation for 2 min at 96° C. was followed by 40 cycles of 96° C. for 20 sec. 67° C. for 15 sec and 72° C. for 1.5 min, and lastly by one cycle of 72° C. for 4 min. The PCR product was digested with EcoRI and cloned into a modified pET28 vector with a His tag in-frame on the 5′ end, which had been digested with Eco72I and EcoRI. The correct construct was confirmed through sequence analysis and transformed into BLR(DE3)pLysS and BLR(DE3)CodonPlus RIL E. coli cells. The amino acid sequence of the recombinant BM31 protein, including His tag, is provided in SEQ ID NO: 179, with the cDNA sequence of the coding region being provided in SEQ ID NO: 169.

H. Expression of Recombinant BM24

The open reading frame of BM-24 (SEQ ID NO: 130) was amplified by PCR using the primers PDM-496 (SEQ ID NO: 161) and PDM-497 (SEQ ID NO: 162). PCR was carried out as described above except that denaturation for 2 min at 96° C. was followed by 40 cycles of 96° C. for 20 sec. 61° C. for 15 sec and 72° C. for 1.5 min, and lastly by one cycle of 72° C. for 4 ml. The PCR product was digested with EcoRI and cloned into a modified pET28 vector with a His tag in-frame on the 5′ end, which had been digested with Eco72I and EcoRI. The correct construct was confirmed through sequence analysis and transformed into BLR(DE3)pLysS and BLR(DE3)CodonPlus RIL E. coli cells. The amino acid sequence of the recombinant BM24 protein, including His tag, is provided in SEQ ID NO: 180, with the cDNA sequence of the coding region being provided in SEQ ID NO: 171.

### EXAMPLE 7

### Identification of Reactive Epitopes in B. microti Antigens

A series of 11 overlapping peptides to the antigen MN-10 (referred to as MN10-1-MN10-11; SEQ ID NO: 191, 188, 187, 186, 185, 184, 183, 182, 181, 190 and 189, respectively) and 11 overlapping peptides to the antigen BMNI-17 (referred to as BMNI17-1-12; SEQ ID NO: 200, 197, 196, 195, 193, 192, 202, 203, 201, 199 and 198, respectively) were prepared as described above, together with a peptide that represents a combination of BMNI17-4 with MN10-8 (SEQ ID NO: 194).

ELISA plates were coated with either the individual peptides, a mix of the peptides BMNI17-4 and MN10-8, or the combination peptide, and incubated with sera taken from patients infected with B. microti. Protein A-HRP was used to bind any IgG antibodies that had been captured by the antigen. Comparison of the peptide-coated ELISA plates to ELISA plates coated with recombinant BMNI-17 or MN-10 showed reactivity to the infected sera in both the recombinant proteins and most of the peptides. However, the peptides did not react with sera taken patients infected with malaria. Peptides that showed good reactivity to Babesia-positive sera and that did not react with malaria-positive sera were tested further. A mix of the peptides BMNI17-4 and MN10-8 was found to show as much reactivity to Babesia-positive sera as the recombinant proteins but no reactivity to malaria-positive sera. The individual peptides, as well as a combination of the reactive peptides, showed low background reactivity to normal human sera. A fusion of the two peptides BMNI17-4 and MN10-8 was prepared and found to possess the same reactivity as the individual peptides. This fusion, together with the peptides BMNI17-4 and MN10-8, may be usefully employed in screening blood for the presence of Babesia infection.

### EXAMPLE 8

### Identification of a Variant Sequence of the Antigen MN-10

Using PCR, a variant of the B. microti antigen MN-10 was isolated that contains a deletion in the region of MN-10 for which diagnostic peptides are being developed. Identification of this variant permits inclusion of other diagnostic peptides in an ELISA assay so that infection with variant forms of B. microti can be detected.

PCR primers were designed from putative conserved regions of the MN10 sequence, to include the repeat regions of the MN 10 sequence and also to include as much of the sequence as possible. The DNASTAR™ package program PrimerSelect™ was used to identify reasonable candidates for primers and primer pairs. Several primer pairs generated by this method were then tested using DNA isolated from Babesia-infected hamster blood. When a primer pair generated an appropriate product, based on accumulation and correct size migration of visible product on agarose gels, a method was optimized for that primer pair. The primer pair was then tested with DNA isolated from known Babesia microti-infected patient samples. Each 100 ul reaction mixture contained the following: 2.5 units of PFUTurbo™ (Stratagene), 1×PFU buffer (Stratagene), 100 uM dNTP mix (GibcoBRL), 500 nM of each primer and 0.5 ul of sample DNA (the amount of template is variable from patient to patient depending on the parasitemia levels of the organism but the detectable range is approximately from 5.0 ng to 5.0 pg). The conditions for amplification were as follows: 94° C. for 5 minutes followed by 45 cycles of 94° C. for 1 minute, 49° C. for 1 minute, 72° C. for 2 minutes and ending with one cycle of 72° C. for 5 minutes.

A variant of the original MN10 sequence was identified using the PCR primers MN10.2 and MN10.5 (SEQ ID NO: 206 and 205, respectively) and the protocol described above on a panel of DNA samples from known Babesia microti-infected patients. Amplification of MN10 sequences from a subset of patients from Nantucket, Mass., generated a truncated version of MN10 containing an approximately 153 base pair (51 amino acids) deletion in the region containing degenerate repeats. The PCR product was cloned and sequenced using conventional methods. The genomic DNA sequence of the MN-1 variant is provided in SEQ ID NO: 204, with the corresponding amino acid sequence being provided in SEQ ID NO: 209.

### EXAMPLE 9

### Identification of Peptide Epitopes of the Antigen MN-10

Using standard techniques, a series of overlapping peptides covering the amino acid sequence of MN-10 (SEQ ID NO: 52) were prepared. The peptide MN10-5 spans the juncture from the conserved region into the degenerate repeat region of MN-10 and the peptide MN10-6 is entirely in the repeat region of MN-10. MN10-5 showed a limited amount of reactivity by ELISA and MN10-6 showed good reactivity by ELISA. To ensure that the immunodominant epitope in the repeat region is represented, a combination peptide of MN10-5 and MN10-6 (referred to as MN10-5/6; SEQ ID NO: 208) was synthesized using conventional methods.

Epitope mapping of MN10 showed that the most immunodominant epitopes are contained within the peptide MN10-8. The deletion of the variant described above occurs in this region, spanning half of MN10-6, all of MN10-7 and MN10-8, and part of MN10-9. A peptide derived from the variant (referred to as MN10-10, SEQ ID NO: 207), spanning the region the deletion was designed and synthesized using conventional methods to be tested for diagnostic use.

### EXAMPLE 10

### Identification of Peptide Epitopes of a Fusion of BMNI-17 and BMNI-20

As discussed above, the antigens BMNI-17 and BMNI-20 are members of the same immunoreactive gene family. More N-terminal sequence information is available for BMNI-20 than for BMNI-17, whereas more C-terminal sequence is available for BMNI-17. A fusion sequence of BMNI-17 and BMNI-20 was therefore designed to give the maximal sequence length. The DNA sequence of this fusion is provided in SEQ ID NO: 210, with the corresponding amino acid sequence being provided in SEQ ID NO: 211.

A series of 10 overlapping peptides to the N-terminal sequence of SEQ ID NO: 211 (SEQ ID NO: 212-221; referred to as BMN N-term 1-BMN N-term 10, respectively), and three overlapping peptides to the C terminal sequence of SEQ ID NO: 211 (SEQ ID NO: 222-224; referred to as BMN C-term 3-BMN C-term 1, respectively) were prepared as described above, and their reactivity with sera from patients infected with B. microti was determined by ELISA as described above in Example 7. High levels of seroreactivity were observed with the peptides BMN N-term 8, BMN N-term 2 and BMN N-term 6 (SEQ ID NO: 219, 213 and 217, respectively).

Although the present invention has been described in some detail by way of illustration and example for purposes of clarity of understanding, changes and modifications can be carried out without departing from the scope of the invention which is intended to be limited only by the scope of the appended claims.

1. An isolated polynucleotide comprising a sequence selected from the group consisting of:
(a) sequences provided in SEQ ID NO: 1-17, 37, 40, 42, 45, 50, 51, 91-119, 128-131, 135, 204 and 210;
(b) complements of the sequences provided in SEQ ID NO: 1-17, 37, 40, 42, 45, 50, 51, 91-119, 128-131, 135, 204 and 210;
(c) sequences that hybridize to a sequence provided in SEQ ID NO: 1-17, 37, 40, 42, 45, 50, 51, 91-119, 128-131, 135, 204 and 210, under moderately stringent conditions;
(d) sequences having at least 75% identity to a sequence of SEQ ID NO: 1-17, 37, 40, 42, 45, 50, 51, 91-119, 128-131, 135, 204 and 210;
(e) sequences having at least 90% identity to a sequence of SEQ ID NO: 1-17, 37, 40, 42, 45, 50, 51, 91-119, 128-131, 135, 204 and 210; and
(f) degenerate variants of a sequence provided in SEQ ID NO: 1-17, 37, 40, 42, 45, 50, 51, 91-119, 128-131, 135, 204 and 210.

2. An isolated polypeptide comprising an amino acid sequence selected from the group consisting of:
(a) sequences encoded by a polynucleotide of claim 1; and
(b) sequences having at least 70% identity to a sequence encoded by a polynucleotide of claim 1;
(c) sequences having at least 90% identity to a sequence encoded by a polynucleotide of claim 1; and
(d) sequences selected from the group consisting of SEQ ID NO: 181-203, 207-209 and 211-224.

3. An isolated antigenic epitope of a B. microti antigen comprising the amino acid sequence -X₁-X₂-X₃-X₄-X₅-Ser-, wherein X₁ is Glu or Gly, X₂ is Ala or Thr, X₃ is Gly or Val, X₄ is Trp or Gly and X₅ is Pro or Ser.

4. An isolated antigenic epitope according to claim 3 wherein X₁ is Glu, X₂ is Ala and X₃ is Gly.

5. An isolated antigenic epitope according to claim 3 wherein X₁ is Gly, X₂ is Thr and X₅ is Pro.

6. An isolated polypeptide comprising at least two contiguous antigenic epitopes according to claim 3.

7. An isolated antigenic epitope of a B. microti antigen comprising an amino acid sequence selected from the group consisting of SEQ ID NOs:36 and 39.

8. An isolated polypeptide comprising at least two contiguous antigenic epitopes according to any one of claims 3 and 7.

9. An expression vector comprising a polynucleotide of claim 1 operably linked to an expression control sequence.

10. A host cell transformed or transfected with an expression vector according to claim 9.

11. An isolated antibody, or antigen-binding fragment thereof, that specifically binds to a polypeptide of claim 2.

12. A fusion protein comprising at least one polypeptide according to claim 2.

13. A fusion protein comprising a polypeptide having an amino acid sequence of SEQ ID NO:32.

14. The fusion protein of claim 13 further comprising a polypeptide having an amino acid sequence of SEQ ID NO:52.

15. A fusion protein comprising at least two antigenic epitopes according to any one of claims 3 and 7.

16. A fusion protein comprising at least one polypeptide according to any one of claims 2, 6 and 8, and at least one antigenic epitope according to any one of claims 3 and 7.

17. An oligonucleotide that hybridizes to a sequence recited in SEQ ID NO: 1-17, 37, 40, 42, 45, 50, 51, 91-119, 128-131, 135, 204 and 210 under moderately stringent conditions.

18. A composition comprising a first component selected from the group consisting of physiologically acceptable carriers and immunostimulants, and a second component selected from the group consisting of:
(a) polypeptides according to any one of claims 2, 6 and 8;
(b) polynucleotides according to claim 1;
(c) antibodies according to claim 11; and
(d) fusion proteins according to any one of claims 13, 16 and 36.

19. A method for stimulating an immune response in a patient, comprising administering to the patient a composition of claim 18.

20. A method for the treatment of B. microti infection in a patient, comprising administering to the patient a composition of claim 18.

21. A method for determining B. microti infection in a patient, comprising the steps of:
(a) obtaining a biological sample from the patient;
(b) contacting the biological sample with an oligonucleotide according to claim 18;
(c) detecting in the sample an amount of a polynucleotide that hybridizes to the oligonucleotide; and
(d) comparing the amount of polynucleotide that hybridizes to the oligonucleotide to a predetermined cut-off value, and therefrom determining B. microti infection in the patient.

22. A diagnostic kit comprising at least one oligonucleotide according—to claim 18.

23. A diagnostic kit comprising at least one antibody according to claim 5 and a detection reagent, wherein the detection reagent comprises a reporter group.

24. A method for detecting B. microti infection in a patient, comprising the steps of:
(a) obtaining a biological sample from the patient;
(b) contacting the biological sample with a binding agent that binds to a polypeptide of claim 2;
(c) detecting in the sample an amount of polypeptide that binds to the binding agent; and
(e) comparing the amount of polypeptide to a predetermined cut-off value and therefrom determining B. microti infection in the patient.

25. A method for detecting B. microti infection in a patient, comprising:
(a) obtaining a sample from the patient;
(b) contacting the sample with at least one antigenic epitope according to any one of claims 3 and 7; and
(c) detecting the presence of antibodies that bind to the antigenic epitope.

26. A method for detecting B. microti infection in a patient, comprising:
(a) obtaining a sample from the patient;
(b) contacting the sample with at least one polypeptide according to any one of claims 2, 6 and 8; and
The (c) detecting the presence of antibodies that bind to the polypeptide.

27. A method for detecting B. microti infection in a patient, comprising:
(a) obtaining a sample from the patient;
(b) contacting the sample with at least one polypeptide according to any one of claims 2, 6 and 8, and at least one antigenic epitope according to any one of claims 3 and 7; and
(c) detecting the presence of antibodies that bind to the polypeptide or antigenic epitope.

28. A method for detecting B. microti infection in a patient, comprising:
(a) obtaining a sample from the patient;
(b) contacting the sample with a fusion protein according to any one of claims 13, 16 and 36, and
(c) detecting the presence of antibodies that bind to the fusion protein.

29. A method of detecting B. microti infection in a biological sample, comprising:
(a) contacting the biological sample with a first binding agent which is capable of binding to a polypeptide according to any one of claims 2, 6 and 8, and a second binding agent which is capable of binding to an antigenic epitope according to any one of claims 3 and 7; and
(b) detecting in the sample a polypeptide that binds to the first binding agent or an antigenic epitope that binds to the second binding agent, thereby detecting B. microti infection in the biological sample.

30. The method of claim 29 wherein the binding agent is a monoclonal antibody.

31. The method of claim 29 wherein the binding agent is a polyclonal antibody.

32. A diagnostic kit comprising
(a) at least one polypeptide according to any one of claims 2, 6 and 8;
(b) a detection reagent.

33. A diagnostic kit comprising:
(a) at least one antigenic epitope according to any one of claims 3 and 7; and
(b) a detection reagent.

34. A diagnostic kit comprising:
(a) at least one antigenic epitope according to any one of claims 3 and 7;
(b) at least one polypeptide according to any one of claims 2, 6 and 8; and
(c) a detection reagent.

35. A fusion protein comprising an amino acid sequence selected from the group consisting of SEQ ID NO: 85, 87, 144 and 211.

36. A diagnostic kit comprising:
(a) at least one fusion protein according to any one of claims 13, 16 and 35; and
(b) a detection reagent.
